SEQUENCE LISTING (298 sequences)

SEQ ID NO: 1 - protein, 135 aa, synthetic construct
  REGION 1..135: note = N-terminal N152 AvrBs3
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 40 aa, synthetic construct
  REGION 1..40: note = 40 AA V2 RR C-terminal
  VARIANT 37..38: note = misc_feature - Xaa can be Arg or His
SIVAQLSRPDPALAALTNDHLVALACLGGRPALDAVXXGL

SEQ ID NO: 3 - protein, 50 aa, synthetic construct
  REGION 1..50: note = 50 AA V2 RR C-terminal
  VARIANT 37..38: note = misc_feature - Xaa can be Arg or His
SIVAQLSRPDPALAALTNDHLVALACLGGRPALDAVXXGLPHAPALIXRT

SEQ ID NO: 4 - protein, 60 aa, synthetic construct
  REGION 1..60: note = 60 AA V2 RR C-terminal
  VARIANT 37..38: note = misc_feature - Xaa can be Arg or His
SIVAQLSRPDPALAALTNDHLVALACLGGRPALDAVXXGLPHAPALIXRTNRRIPERTSH

SEQ ID NO: 5 - protein, 34 aa, synthetic construct
  REGION 1..34: note = AvrBs3 Repeat DD
  VARIANT 12..13: note = misc_feature - Xaa can be any naturally occuring amino acid or can be absent
LTPDQVVAIASXXGGKQALETVQRLLPVLCQDHG

SEQ ID NO: 6 - protein, 34 aa, synthetic construct
  REGION 1..34: note = AvrBs3 Repeat DD + A23
  VARIANT 12..13: note = misc_feature - Xaa can be any naturally occuring amino acid or can be absent
LTPDQVVAIASXXGGKQALETVQALLPVLCQDHG

SEQ ID NO: 7 - protein, 34 aa, synthetic construct
  REGION 1..34: note = AvrBs3 Repeat DD + Q24
  VARIANT 12..13: note = misc_feature - Xaa can be any naturally occuring amino acid or can be absent
LTPDQVVAIASXXGGKQALETVQQLLPVLCQDHG

SEQ ID NO: 8 - protein, 34 aa, synthetic construct
  REGION 1..34: note = AvrBs3 Repeat DD + L6
  VARIANT 12..13: note = misc_feature - Xaa can be any naturally occuring amino acid or can be absent
LTPDQLVAIASXXGGKQALETVQRLLPVLCQDHG

SEQ ID NO: 9 - protein, 34 aa, synthetic construct
  REGION 1..34: note = AvrBs3 Repeat DD + M6
  VARIANT 12..13: note = misc_feature - Xaa can be any naturally occuring amino acid or can be absent
LTPDQMVAIASXXGGKQALETVQRLLPVLCQDHG

SEQ ID NO: 10 - protein, 34 aa, synthetic construct
  REGION 1..34: note = AvrBs3 Repeat DD + Q34
  VARIANT 12..13: note = misc_feature - Xaa can be any naturally occuring amino acid or can be absent
LTPDQVVAIASXXGGKQALETVQRLLPVLCQDQG

SEQ ID NO: 11 - protein, 34 aa, synthetic construct
  REGION 1..34: note = AvrBs3 Repeat DD + L3
  VARIANT 12..13: note = misc_feature - Xaa can be any naturally occuring amino acid or can be absent
LTLDQVVAIASXXGGKQALETVQRLLPVLCQDHG

SEQ ID NO: 12 - protein, 7 aa, synthetic construct
  REGION 1..7: note = NLS SV40
PKKKRKV

SEQ ID NO: 13 - protein, 9 aa, synthetic construct
  REGION 1..9: note = C-Myc NLS
PAAKKKKLD

SEQ ID NO: 14 - protein, 16 aa, synthetic construct
  REGION 1..16: note = Nucleoplasmin NLS
KRPAATKKAGQAKKKK

SEQ ID NO: 15 - protein, 6 aa, synthetic construct
  REGION 1..6: note = VACM-1/CUL5 NLS
PKLKRQ

SEQ ID NO: 16 - protein, 4 aa, synthetic construct
  REGION 1..4: note = CXCR4 NLS
RPRK

SEQ ID NO: 17 - protein, 8 aa, synthetic construct
  REGION 1..8: note = VP1 NLS
RRARRPRG

SEQ ID NO: 18 - protein, 21 aa, synthetic construct
  REGION 1..21: note = 58BP1 NLS
GKRKLITSEEERSPAKRGRKS

SEQ ID NO: 19 - protein, 22 aa, synthetic construct
  REGION 1..22: note = ING4 NLS
KGKKGRTQKEKKAARARSKGKN

SEQ ID NO: 20 - protein, 28 aa, synthetic construct
  REGION 1..28: note = IER5 NLS
RKRCAAGVGGGPAGCPAPGSTPLKKPRR

SEQ ID NO: 21 - protein, 36 aa, synthetic construct
  REGION 1..36: note = ERK5 NLS
RKPVTAQERQREREEKRRRRQERAKEREKRRQERER

SEQ ID NO: 22 - protein, 28 aa, synthetic construct
  REGION 1..28: note = cytochrome c oxidase subunit 8A mitochondrial addressing signals
SVLTPLLLRGLTGSARRLPVPRAKIHSL

SEQ ID NO: 23 - protein, 29 aa, synthetic construct
  REGION 1..29: note = superoxide dismutase 2 mitochondrial addressing signals
LSRAVCGTSRQLAPVLGYLGSRQKHSLPD

SEQ ID NO: 24 - protein, 34 aa, synthetic construct
  REGION 1..34: note = TALE Repeat DD (R) NI
LTPDQVVAIASNIGGKQALETVQRLLPVLCQAHG

SEQ ID NO: 25 - protein, 34 aa, synthetic construct
  REGION 1..34: note = TALE Repeat DD (R) HD
LTPDQVVAIASHDGGKQALETVQRLLPVLCQDHG

SEQ ID NO: 26 - protein, 34 aa, synthetic construct
  REGION 1..34: note = TALE Repeat DD (R) NN
LTPDQVVAIASNNGGKQALETVQRLLPVLCQDHG

SEQ ID NO: 27 - protein, 34 aa, synthetic construct
  REGION 1..34: note = TALE Repeat DD (R) NG
LTPQDVVAIASNGGGKQALETVQRLLPVLCQDHG

SEQ ID NO: 28 - protein, 40 aa, synthetic construct
  REGION 1..40: note = 40 AA AvrBs3 C-terminal
SIVAQLSRPDPALAALTNDHLVALACLGGRPALDAVKKGL

SEQ ID NO: 29 - protein, 50 aa, synthetic construct
  REGION 1..50: note = 50 AA AvrBs3 C-terminal
SIVAQLSRPDPALAALTNDHLVALACLGGRPALDAVKKGLPHAPALIRRT

SEQ ID NO: 30 - protein, 60 aa, synthetic construct
  REGION 1..60: note = 60 AA AvrBs3 C-terminal
SIVAQLSRPDPALAALTNDHLVALACLGGRPALDAVKKGLPHAPALIRRTNRRIPERTSH

SEQ ID NO: 31 - protein, 34 aa, synthetic construct
  REGION 1..34: note = TALE Repeat AvrBs3 NI canonical
MTPEQVVAIASNIGGKQALETVQALLPVLCQAHG

SEQ ID NO: 32 - protein, 34 aa, synthetic construct
  REGION 1..34: note = TALE Repeat AvrBs3 HD canonical
MTPEQVVAIASHDGGKQALETVQRLLPVLCQAHG

SEQ ID NO: 33 - protein, 34 aa, synthetic construct
  REGION 1..34: note = TALE Repeat AvrBs3 NN canonical
MTPQQVVAIASNNGGKQALETVQRLLPVLCQAHG

SEQ ID NO: 34 - protein, 34 aa, synthetic construct
  REGION 1..34: note = TALE Repeat AvrBs3 NG canonical
MTPQQVVAIASNGGGKQALETVQRLLPVLCQAHG

SEQ ID NO: 35 - protein, 4 aa, synthetic construct
  REGION 1..4: note = 1dnpA_1 linker
DSVI

SEQ ID NO: 36 - protein, 4 aa, synthetic construct
  REGION 1..4: note = 1d8cA_2 linker
IVEA

SEQ ID NO: 37 - protein, 4 aa, synthetic construct
  REGION 1..4: note = 1ckqA_3 linker
LEGS

SEQ ID NO: 38 - protein, 4 aa, synthetic construct
  REGION 1..4: note = 1sbp_1 linker
YTST

SEQ ID NO: 39 - protein, 5 aa, synthetic construct
  REGION 1..5: note = 1ev7A_1 linker
LQENL

SEQ ID NO: 40 - protein, 5 aa, synthetic construct
  REGION 1..5: note = 1alo_3 linker
VGRQP

SEQ ID NO: 41 - protein, 5 aa, synthetic construct
  REGION 1..5: note = 1amf_1 linker
LGNSL

SEQ ID NO: 42 - protein, 6 aa, synthetic construct
  REGION 1..6: note = 1adjA_3 linker
LPEEKG

SEQ ID NO: 43 - protein, 6 aa, synthetic construct
  REGION 1..6: note = 1fcdC_1 linker
QTYQPA

SEQ ID NO: 44 - protein, 6 aa, synthetic construct
  REGION 1..6: note = 1al3_2 linker
FSHSTT

SEQ ID NO: 45 - protein, 7 aa, synthetic construct
  REGION 1..7: note = 1g3p_1 linker
GYTYINP

SEQ ID NO: 46 - protein, 7 aa, synthetic construct
  REGION 1..7: note = 1acc_3 linker
LTKYKSS

SEQ ID NO: 47 - protein, 8 aa, synthetic construct
  REGION 1..8: note = 1ahjB_1 linker
SRPSESEG

SEQ ID NO: 48 - protein, 8 aa, synthetic construct
  REGION 1..8: note = 1acc_1 linker
PELKQKSS

SEQ ID NO: 49 - protein, 8 aa, synthetic construct
  REGION 1..8: note = 1af7_1 linker
LTTNLTAF

SEQ ID NO: 50 - protein, 9 aa, synthetic construct
  REGION 1..9: note = 1heiA_1 linker
TATPPGSVT

SEQ ID NO: 51 - protein, 9 aa, synthetic construct
  REGION 1..9: note = 1bia_2 linker
LDNFINRPV

SEQ ID NO: 52 - protein, 9 aa, synthetic construct
  REGION 1..9: note = 1igtB_1 linker
VSSAKTTAP

SEQ ID NO: 53 - protein, 10 aa, synthetic construct
  REGION 1..10: note = 1nfkA_1 linker
DSKAPNASNL

SEQ ID NO: 54 - protein, 10 aa, synthetic construct
  REGION 1..10: note = 1au7A_1 linker
KRRTTISIAA

SEQ ID NO: 55 - protein, 11 aa, synthetic construct
  REGION 1..11: note = 1bpoB_1 linker
PVKMFDRHSSL

SEQ ID NO: 56 - protein, 11 aa, synthetic construct
  REGION 1..11: note = 1b0pA_2 linker
APAETKAEPMT

SEQ ID NO: 57 - protein, 14 aa, synthetic construct
  REGION 1..14: note = 1c05A_2 linker
YTRLPERSELPAEI

SEQ ID NO: 58 - protein, 14 aa, synthetic construct
  REGION 1..14: note = 1gcb_1 linker
VSTDSTPVTNQKSS

SEQ ID NO: 59 - protein, 14 aa, synthetic construct
  REGION 1..14: note = 1bt3A_1 linker
YKLPAVTTMKVRPA

SEQ ID NO: 60 - protein, 15 aa, synthetic construct
  REGION 1..15: note = 1b3oB_2 linker
IARTDLKKNRDYPLA

SEQ ID NO: 61 - protein, 21 aa, synthetic construct
  REGION 1..21: note = 16vpA_6 linker
TEEPGAPLTTPPTLHGNQARA

SEQ ID NO: 62 - protein, 21 aa, synthetic construct
  REGION 1..21: note = 1dhx_1 linker
ARFTLAVGDNRVLDMASTYFD

SEQ ID NO: 63 - protein, 26 aa, synthetic construct
  REGION 1..26: note = 1b8aA_1 linker
IVVLNRAETPLPLDPTGKVKAELDTR

SEQ ID NO: 64 - protein, 28 aa, synthetic construct
  REGION 1..28: note = 1qu6A_1 linker
ILNKEKKAVSPLLLTTTNSSEGLSMGNY

SEQ ID NO: 65 - protein, 20 aa, synthetic construct
  REGION 1..20: note = NFS1 linker
GSDITKSKISEKMKGQGPSG

SEQ ID NO: 66 - protein, 23 aa, synthetic construct
  REGION 1..23: note = NFS2 linker
GSDITKSKISEKMKGLGPDGRKA

SEQ ID NO: 67 - protein, 10 aa, synthetic construct
  REGION 1..10: note = CFS1 linker
SLTKSKISGS

SEQ ID NO: 68 - protein, 32 aa, synthetic construct
  REGION 1..32: note = RM2 linker
AAGGSALTAGALSLTAGALSLTAGALSGGGGS

SEQ ID NO: 69 - protein, 27 aa, synthetic construct
  REGION 1..27: note = BQY linker
AAGASSVSASGHIAPLSLPSSPPSVGS

SEQ ID NO: 70 - protein, 5 aa, synthetic construct
  REGION 1..5: note = QGPSG linker
QGPSG

SEQ ID NO: 71 - protein, 8 aa, synthetic construct
  REGION 1..8: note = LGPDGRKA linker
LGPDGRKA

SEQ ID NO: 72 - protein, 15 aa, synthetic construct
  REGION 1..15: note = TAL1 linker
SGGSGSNVGSGSGSG

SEQ ID NO: 73 - protein, 20 aa, synthetic construct
  REGION 1..20: note = TAL2 linker
SGGSGSLTTNLTAFSGSGSG

SEQ ID NO: 74 - protein, 22 aa, synthetic construct
  REGION 1..22: note = TAL3 linker
SGGSGSKRRTTISIAASGSGSG

SEQ ID NO: 75 - protein, 17 aa, synthetic construct
  REGION 1..17: note = TAL4 linker
SGGSGSVGRQPSGSGSG

SEQ ID NO: 76 - protein, 26 aa, synthetic construct
  REGION 1..26: note = TAL5 linker
SGGSGSYTRLPERSELPAEISGSGSG

SEQ ID NO: 77 - protein, 38 aa, synthetic construct
  REGION 1..38: note = TAL6 linker
SGGSGSIVVLNRAETPLPLDPTGKVKAELDTRSGSGSG

SEQ ID NO: 78 - protein, 21 aa, synthetic construct
  REGION 1..21: note = TAL7 linker
SGGSGSTATPPGSVTSGSGSG

SEQ ID NO: 79 - protein, 21 aa, synthetic construct
  REGION 1..21: note = TAL8 linker
SGGSGSLDNFINRPVSGSGSG

SEQ ID NO: 80 - protein, 21 aa, synthetic construct
  REGION 1..21: note = TAL9 linker
SGGSGSVSSAKTTAPSGSGSG

SEQ ID NO: 81 - protein, 22 aa, synthetic construct
  REGION 1..22: note = TAL10 linker
SGGSGSDSKAPNASNLSGSGSG

SEQ ID NO: 82 - protein, 23 aa, synthetic construct
  REGION 1..23: note = TAL11 linker
SGGSGSPVKMFDRHSSLSGSGSG

SEQ ID NO: 83 - protein, 23 aa, synthetic construct
  REGION 1..23: note = TAL12 linker
SGGSGSAPAETKAEPMTSGSGSG

SEQ ID NO: 84 - protein, 26 aa, synthetic construct
  REGION 1..26: note = TAL13 linker
SGGSGSVSTDSTPVTNQKSSSGSGSG

SEQ ID NO: 85 - protein, 16 aa, synthetic construct
  REGION 1..16: note = TAL14 linker
SGGSGSDSVISGSGSG

SEQ ID NO: 86 - protein, 33 aa, synthetic construct
  REGION 1..33: note = TAL15 linker
SGGSGSARFTLAVGDNRVLDMASTYFDSGSGSG

SEQ ID NO: 87 - protein, 17 aa, synthetic construct
  REGION 1..17: note = TAL16 linker
SGGSGSLQENLSGSGSG

SEQ ID NO: 88 - protein, 19 aa, synthetic construct
  REGION 1..19: note = TAL17 linker
SGGSGSGYTYINPSGSGSG

SEQ ID NO: 89 - protein, 26 aa, synthetic construct
  REGION 1..26: note = TAL18 linker
SGGSGSYKLPAVTTMKVRPASGSGSG

SEQ ID NO: 90 - protein, 16 aa, synthetic construct
  REGION 1..16: note = TAL19 linker
SGGSGSLEGSSGSGSG

SEQ ID NO: 91 - protein, 16 aa, synthetic construct
  REGION 1..16: note = TAL20 linker
SGGSGSIVEASGSGSG

SEQ ID NO: 92 - protein, 18 aa, synthetic construct
  REGION 1..18: note = TAL21 linker
SGGSGSQTYQPASGSGSG

SEQ ID NO: 93 - protein, 27 aa, synthetic construct
  REGION 1..27: note = TAL22 linker
SGGSGSIARTDLKKNRDYPLASGSGSG

SEQ ID NO: 94 - protein, 18 aa, synthetic construct
  REGION 1..18: note = TAL23 linker
SGGSGSLPEEKGSGSGSG

SEQ ID NO: 95 - protein, 16 aa, synthetic construct
  REGION 1..16: note = TAL24 linker
SGGSGSYTSTSGSGSG

SEQ ID NO: 96 - protein, 20 aa, synthetic construct
  REGION 1..20: note = TAL25 linker
SGGSGSSRPSESEGSGSGSG

SEQ ID NO: 97 - protein, 17 aa, synthetic construct
  REGION 1..17: note = TAL26 linker
SGGSGSLGNSLSGSGSG

SEQ ID NO: 98 - protein, 19 aa, synthetic construct
  REGION 1..19: note = TAL27 linker
SGGSGSLTKYKSSSGSGSG

SEQ ID NO: 99 - protein, 33 aa, synthetic construct
  REGION 1..33: note = TAL28 linker
SGGSGSTEEPGAPLTTPPTLHGNQARASGSGSG

SEQ ID NO: 100 - protein, 18 aa, synthetic construct
  REGION 1..18: note = TAL29 linker
SGGSGSFSHSTTSGSGSG

SEQ ID NO: 101 - protein, 20 aa, synthetic construct
  REGION 1..20: note = TAL30 linker
SGGSGSPELKQKSSSGSGSG

SEQ ID NO: 102 - protein, 40 aa, synthetic construct
  REGION 1..40: note = TAL31 linker
SGGSGSILNKEKKAVSPLLLTTTNSSEGLSMGNYSGSGSG

SEQ ID NO: 103 - protein, 31 aa, synthetic construct
  REGION 1..31: note = TAL32 linker
ELAEFHARYADLLLRDLRERPVSLVRGPDSG

SEQ ID NO: 104 - protein, 31 aa, synthetic construct
  REGION 1..31: note = TAL33 linker
ELAEFHARPDPLLLRDLRERPVSLVRGLGSG

SEQ ID NO: 105 - protein, 26 aa, synthetic construct
  REGION 1..26: note = TAL34 linker
ELAEFHARYADLLLRDLRERSGSGSG

SEQ ID NO: 106 - protein, 31 aa, synthetic construct
  REGION 1..31: note = TAL35 linker
DIFDYYAGVAEVMLGHIAGRPATRKRWPNSG

SEQ ID NO: 107 - protein, 31 aa, synthetic construct
  REGION 1..31: note = TAL36 linker
DIFDYYAGPDPVMLGHIAGRPATRKRWLGSG

SEQ ID NO: 108 - protein, 26 aa, synthetic construct
  REGION 1..26: note = TAL37 linker
DIFDYYAGVAEVMLGHIAGRSGSGSG

SEQ ID NO: 109 - protein, 196 aa, Flavobacterium okeanokoites
  REGION 1..196: note = Fok-1 catalytic domain (196 AA)
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 110 - protein, 715 aa, Ophiostoma novo-ulmi
  REGION 1..715: note = I-Onu1
MENNIKLENSCCALNKNKSNIFNKYINNKYKLVPFKTLVNYVNEPRYIPSEFKEWNNSIYYFNFNNIKNLPVYDINLNKLLKSYFDLYFISKNKNNKFISIIKKKQRYSLNKIFISKADLKHTSSKIIITIYIFNRERIILIKNLIFLYSLHFKTKSYLEKNKNLFFFESLKKKLNNKYEIFNKLKLNFNLNNLKFKDIMLYKLSKLLSKFYNKKVEFNIINLNSYKYNSDILTDIFKKKVVNPNSKLIKIMKFIGKKSLRASIGKTGDNYMDKTRISKSINYDLIPNKYKNLNISLIIENINFNETIKNIYNISNDTNENIIYNSIKYKLVVGVRLAIKGRLTKRYRADRSKLYSKTVGNLQNIDSSFKGLSSKLYRNKLNSNMQYTLDVYKRHVGAYAVKGWISGRSYSTSAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 111 - protein, 163 aa, Chlamydomonas reinhardtii
  REGION 1..163: note = I-CreI
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 112 - protein, 266 aa, Bacillus mojavensis
  REGION 1..266: note = I-BmoI
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIIDGTEYESATEASRQLNVVPATILHRIKSKNEKYSGYFYK

SEQ ID NO: 113 - protein, 174 aa, Bacillus sp.
  REGION 1..174: note = I-HmuI
MEWKDIKGYEGHYQVSNTGEVYSIKSGKTLKHQIPKDGYHRIGLFKGGKGKTFQVHRLVAIHFCEGYEEGLVVDHKDGNKDNNLSTNLRWVTQKINVENQMSRGTLNVSKAQQIAKIKNQKPIIVISPDGIEKEYPSTKCACEELGLTRGKVTDVLKGHRIHHKGYTFRYKLNG

SEQ ID NO: 114 - protein, 245 aa, Enterobacteria phage T4
  REGION 1..245: note = I-TevI
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISCDGVIFDCAADAARHFKISSGLVTYRVKSDKWNWFYINA

SEQ ID NO: 115 - protein, 199 aa, Clostridium sp.
  REGION 1..199: note = Clo51
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 116 - protein, 274 aa, Nostoc sp.
  REGION 1..274: note = NucA
MGICGKLGVAALVALIVGCSPVQSQVPPLTELSPSISVHLLLGNPSGATPTKLTPDNYLMVKNQYALSYNNSKGTANWVAWQLNSSWLGNAERQDNFRPDKTLPAGWVRVTPSMYSGSGYDRGHIAPSADRTKTTEDNAATFLMTNMMPQTPDNNRNTWGNLEDYCRELVSQGKELYIVAGPNGSLGKPLKGKVTVPKSTWKIVVVLDSPGSGLEGITANTRVIAVNIPNDPELNNDWRAYKVSVDELESLTGYDFLSNVSPNIQTSIESKVDN

SEQ ID NO: 117 - protein, 671 aa, Homo sapiens
  REGION 1..671: note = Metnase
MAEFKEKPEAPTEQLDVACGQENLPVGAWPPGAAPAPFQYTPDHVVGPGADIDPTQITFPGCICVKTPCLPGTCSCLRHGENYDDNSCLRDIGSGGKYAEPVFECNVLCRCSDHCRNRVVQKGLQFHFQVFKTHKKGWGLRTLEFIPKGRFVCEYAGEVLGFSEVQRRIHLQTKSDSNYIIAIREHVYNGQVMETFVDPTYIGNIGRFLNHSCEPNLLMIPVRIDSMVPKLALFAAKDIVPEEELSYDYSGRYLNLTVSEDKERLDHGKLRKPCYCGAKSCTAFLPFDSSLYCPVEKSNISCGNEKEPSMCGSAPSVFPSCKRLTLETMKMMLDKKQIRAIFLFEFKMGRKAAETTRNINNAFGPGTANERTVQWWFKKFCKGDESLEDEERSGRPSEVDNDQLRAIIEADPLTTTREVAEELNVNHSTVVRHLKQIGKVKKLDKWVPHELTENQKNRRFEVSSSLILRNHNEPFLDRIVTCDEKWILYDNRRRSAQWLDQEEAPKHFPKPILHPKKVMVTIWWSAAGLIHYSFLNPGETITSEKYAQEIDEMNQKLQRLQLALVNRKGPILLHDNARPHVAQPTLQKLNELGYEVLPHPPYSPDLLPTNYHVFKHLNNFLQGKRFHNQQDAENAFQEFVESQSTDFYATGINQLISRWQKCVDCNGSYFD

SEQ ID NO: 118 - protein, 279 aa, Homo sapiens
  REGION 1..279: note = Human TREX2
MGRAGSPLPRSSWPRMDDCGSRSRCSPTLCSSLRTCYPRGNITMSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 119 - protein, 829 aa, Homo sapiens
  REGION 1..829: note = Human DNA2
FAIPASRMEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 120 - protein, 490 aa, Human herpesvirus 2
  REGION 1..490: note = VP16
MDLLVDDLFADRDGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 121 - protein, 167 aa, Escherichia coli
  REGION 1..167: note = Cytidine deaminase:tRNA-specific adenosine deaminase (TadA)-P68398
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 122 - protein, 229 aa, Rattus norvegicus [sequence omitted]

SEQ ID NO: 123 - protein, 280 aa, Sus scrofa
  REGION 1..280: note = Cytidine deaminase: DNA dC->dU-editing enzyme APOBEC-3B
MDPQRLRQWPGPGPASRGGYGQRPRIRNPEEWFHELSPRTFSFHFRNLRFASGRNRSYICCQVEGKNCFFQGIFQNQVPPDPPCHAELCFLSWFQSWGLSPDEHYYVTWFISWSPCCECAAKVAQFLEENRNVSLSLSAARLYYFWKSESREGLRRLSDLGAQVGIMSFQDFQHCWNNFVHNLGMPFQPWKKLHKNYQRLVTELKQILREEPATYGSPQAQGKVRIGSTAAGLRHSHSHTRSEAHLRPNHSSRQHRILNPPREARARTCVLVDASWICYR

SEQ ID NO: 124 - protein, 192 aa, Alligator mississippiensis
  REGION 1..192: note = Cytidine deaminase: C->U-editing enzyme APOBEC-1
MADSSEKMRGQYISRDTFEKNYKPIDGTKEAHLLCEIKWGKYGKPWLHWCQNQRMNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRLLDIFWESKCRSPNPW

SEQ ID NO: 125 - protein, 236 aa, Pongo pygmaeus
  REGION 1..236: note = Cytidine deaminase: C->U-editing enzyme APOBEC-1
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 126 - protein, 198 aa, Homo sapiens [sequence omitted]

SEQ ID NO: 127 - protein, 190 aa, Homo sapiens
  REGION 1..190: note = Cytidine deaminase: DNA dC->dU-editing enzyme APOBEC-3C - Q9NRW3
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 128 - protein, 386 aa, Homo sapiens
  REGION 1..386: note = Cytidine deaminase: DNA dC->dU-editing enzyme APOBEC-3D isoform 1 - Q96AK3
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 129 - protein, 373 aa, Homo sapiens
  REGION 1..373: note = DNA dC->dU-editing enzyme APOBEC-3F isoform a - Q8IUX4
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 130 - protein, 384 aa, Homo sapiens
  REGION 1..384: note = Cytidine deaminase: DNA dC->dU-editing enzyme APOBEC-3G isoform 1 - Q9HC16
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 131 - protein, 301 aa, Arabidopsis thaliana
  REGION 1..301: note = Cytidine deaminase: cytidine deaminase 1
MDKPSFVIQSKEAESAAKQLGVSVIQLLPSLVKPAQSYARTPISKFNVAVVGLGSSGRIFLGVNVEFPNLPLHHSIHAEQFLVTNLTLNGERHLNFFAVSAAPCGHCRQFLQEIRDAPEIKILITDPNNSADSDSAADSDGFLRLGSFLPHRFGPDDLLGKDHPLLLESHDNHLKISDLDSICNGNTDSSADLKQTALAAANRSYAPYSLCPSGVSLVDCDGKVYRGWYMESAAYNPSMGPVQAALVDYVANGGGGGYERIVGAVLVEKEDAVVRQEHTARLLLETISPKCEFKVFHCYEA

SEQ ID NO: 132 - protein, 691 aa, Moloney murine leukemia virus
  REGION 1..691: note = reverse transcriptase p80 RT [Moloney murine leukemia virus] - P03355
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFE

SEQ ID NO: 133 - protein, 500 aa, Mycobacterium sp. [sequence omitted]

SEQ ID NO: 134 - protein, 108 aa, Burkholderia cenocepacia
  REGION 1..108: note = DddA N-half
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 135 - protein, 30 aa, Burkholderia cenocepacia
  REGION 1..30: note = DddA C-half
AIPVKRGATGETKVFTGNSNSPKSPTKGGC

SEQ ID NO: 136 - protein, 82 aa, Bacillus phage PBS2
  REGION 1..82: note = canonical UGI (uracil-DNA-glycosylase inhibitor)
NLSDIIEKETGKQLVIQESILMLPEEVEEVIGNKPESDILVHTAYDESTDENVMLLTSDAPEYKPWALVIQDSNGENKIKML

SEQ ID NO: 137 - protein, 596 aa, Homo sapiens
  REGION 1..596: note = (subdomain of) DNA topoisomerase 1
DGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 138 - protein, 726 aa, synthetic construct
  REGION 1..726: note = TALE V2 CTLA4 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 139 - protein, 726 aa, synthetic construct
  REGION 1..726: note = TALE V2 CTLA4 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 140 - protein, 726 aa, synthetic construct
  REGION 1..726: note = TALE V2 CISH R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 141 - protein, 726 aa, synthetic construct
  REGION 1..726: note = TALE V2 CISH L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 142 - protein, 726 aa, synthetic construct
  REGION 1..726: note = TALE V2 LAG3 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 143 - protein, 726 aa, synthetic construct
  REGION 1..726: note = TALE V2 LAG3 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 144 - protein, 726 aa, synthetic construct
  REGION 1..726: note = TALE V2 TGFBRII R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 145 - protein, 726 aa, synthetic construct
  REGION 1..726: note = TALE V2 TGFBRII L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 146 - protein, 726 aa, synthetic construct
  REGION 1..726: note = TALE V2 CCR5 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 147 - protein, 726 aa, synthetic construct
  REGION 1..726: note = TALE V2 CCR5 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 148 - protein, 726 aa, synthetic construct
  REGION 1..726: note = TALE V2 B2m L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 149 - protein, 726 aa, synthetic construct
  REGION 1..726: note = TALE V2 B2m R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 150 - protein, 726 aa, synthetic construct
  REGION 1..726: note = TALE V2 B2m-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 151 - protein, 726 aa, synthetic construct
  REGION 1..726: note = TALE V2 B2m-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 152 - protein, 726 aa, synthetic construct
  REGION 1..726: note = TALE V2 TCR&#945; L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 153 - protein, 726 aa, synthetic construct
  REGION 1..726: note = TALE V2 TCR&#945; R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 154 - protein, 726 aa, synthetic construct
  REGION 1..726: note = TALE V2 PD1 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 155 - protein, 726 aa, synthetic construct
  REGION 1..726: note = TALE V2 PD1 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 156 - protein, 726 aa, synthetic construct
  REGION 1..726: note = TALE V2 PIK3CDex8 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 157 - protein, 726 aa, synthetic construct
  REGION 1..726: note = TALE V2 PIK3CDex8 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 158 - protein, 726 aa, synthetic construct
  REGION 1..726: note = TALE V2 PIK3CDex17 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 159 - protein, 726 aa, synthetic construct
  REGION 1..726: note = TALE V2 PIK3CDex17 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 160 - protein, 726 aa, synthetic construct
  REGION 1..726: note = TALE V2 S100A9 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 161 - protein, 726 aa, synthetic construct
  REGION 1..726: note = TALE V2 S100A9 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 162 - protein, 726 aa, synthetic construct
  REGION 1..726: note = TALE V2 AAVS1 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 163 - protein, 726 aa, synthetic construct
  REGION 1..726: note = TALE V2 AAVS1 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 164 - protein, 726 aa, synthetic construct
  REGION 1..726: note = TALE V2 CD52 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 165 - protein, 726 aa, synthetic construct
  REGION 1..726: note = TALE V2 CD52 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 166 - protein, 726 aa, synthetic construct
  REGION 1..726: note = TALE V2 TCR&#945;-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 167 - protein, 726 aa, synthetic construct
  REGION 1..726: note = TALE V2 TCR&#945;-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 168 - protein, 726 aa, synthetic construct
  REGION 1..726: note = TALE V2 TGFBRII-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRS

SEQ ID NO: 169 - protein, 726 aa, synthetic construct
  REGION 1..726: note = TALE V2 TGFBRII-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 170 - protein, 726 aa, synthetic construct
  REGION 1..726: note = TALE V2 TGFBRII-3 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRS

SEQ ID NO: 171 - protein, 726 aa, synthetic construct
  REGION 1..726: note = TALE V2 TGFBRII-3 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRS

SEQ ID NO: 172 - protein, 726 aa, synthetic construct
  REGION 1..726: note = TALE V2 TGFBRII-4 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 173 - protein, 726 aa, synthetic construct
  REGION 1..726: note = TALE V2 TGFBRII-4 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRS

SEQ ID NO: 174 - protein, 925 aa, synthetic construct
  REGION 1..925: note = TALEN V2 CTLA4 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 175 - protein, 925 aa, synthetic construct
  REGION 1..925: note = TALEN V2 CTLA4 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 176 - protein, 925 aa, synthetic construct
  REGION 1..925: note = TALEN V2 CISH R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 177 - protein, 925 aa, synthetic construct
  REGION 1..925: note = TALEN V2 CISH L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 178 - protein, 925 aa, synthetic construct
  REGION 1..925: note = TALEN V2 LAG3 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 179 - protein, 925 aa, synthetic construct
  REGION 1..925: note = TALEN V2 LAG3 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 180 - protein, 925 aa, synthetic construct
  REGION 1..925: note = TALEN V2 TGFBRII R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 181 - protein, 925 aa, synthetic construct
  REGION 1..925: note = TALEN V2 TGFBRII L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 182 - protein, 925 aa, synthetic construct
  REGION 1..925: note = TALEN V2 CCR5 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 183 - protein, 925 aa, synthetic construct
  REGION 1..925: note = TALEN V2 CCR5 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 184 - protein, 925 aa, synthetic construct
  REGION 1..925: note = TALEN V2 B2m L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 185 - protein, 925 aa, synthetic construct
  REGION 1..925: note = TALEN V2 B2m R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 186 - protein, 925 aa, synthetic construct
  REGION 1..925: note = TALEN V2 B2m-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 187 - protein, 925 aa, synthetic construct
  REGION 1..925: note = TALEN V2 B2m-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 188 - protein, 925 aa, synthetic construct
  REGION 1..925: note = TALEN V2 TCR&#945; L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 189 - protein, 925 aa, synthetic construct
  REGION 1..925: note = TALEN V2 TCR&#945; R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 190 - protein, 925 aa, synthetic construct
  REGION 1..925: note = TALEN V2 PD1 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 191 - protein, 925 aa, synthetic construct
  REGION 1..925: note = TALEN V2 PD1 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 192 - protein, 925 aa, synthetic construct
  REGION 1..925: note = TALEN V2 PIK3CDex8 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 193 - protein, 925 aa, synthetic construct
  REGION 1..925: note = TALEN V2 PIK3CDex8 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 194 - protein, 925 aa, synthetic construct
  REGION 1..925: note = TALEN V2 PIK3CDex17 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 195 - protein, 925 aa, synthetic construct
  REGION 1..925: note = TALEN V2 PIK3CDex17 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 196 - protein, 925 aa, synthetic construct
  REGION 1..925: note = TALEN V2 S100A9 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 197 - protein, 925 aa, synthetic construct
  REGION 1..925: note = TALEN V2 S100A9 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 198 - protein, 925 aa, synthetic construct
  REGION 1..925: note = TALEN V2 AAVS1 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 199 - protein, 925 aa, synthetic construct
  REGION 1..925: note = TALEN V2 AAVS1 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 200 - protein, 925 aa, synthetic construct
  REGION 1..925: note = TALEN V2 CD52 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 201 - protein, 925 aa, synthetic construct
  REGION 1..925: note = TALEN V2 CD52 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 202 - protein, 925 aa, synthetic construct
  REGION 1..925: note = TALEN V2 TCR&#945;-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 203 - protein, 925 aa, synthetic construct
  REGION 1..925: note = TALEN V2 TCR&#945;-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 204 - protein, 925 aa, synthetic construct
  REGION 1..925: note = TALEN V2 TGFBRII-2 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 205 - protein, 925 aa, synthetic construct
  REGION 1..925: note = TALEN V2 TGFBRII-2 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 206 - protein, 925 aa, synthetic construct
  REGION 1..925: note = TALEN V2 TGFBRII-3 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 207 - protein, 925 aa, synthetic construct
  REGION 1..925: note = TALEN V2 TGFBRII-3 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 208 - protein, 925 aa, synthetic construct
  REGION 1..925: note = TALEN V2 TGFBRII-4 L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 209 - protein, 925 aa, synthetic construct
  REGION 1..925: note = TALEN V2 TGFBRII-4 R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 210 - protein, 925 aa, synthetic construct
  REGION 1..925: note = CS1 TALEN Left V0 pCLS32855
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 211 - protein, 925 aa, synthetic construct
  REGION 1..925: note = CS1 TALEN right V0 pCLS31911
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 212 - protein, 925 aa, synthetic construct
  REGION 1..925: note = CS1 TALENV0.1 HH left pCLS33633
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVHHGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 213 - protein, 925 aa, synthetic construct
  REGION 1..925: note = CS1 TALEN right V0.1 HH pCLS33634
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVHHGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 214 - protein, 925 aa, synthetic construct
  REGION 1..925: note = CS1 TALEN V0.2 RR left pCLS33943
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 215 - protein, 925 aa, synthetic construct
  REGION 1..925: note = CS1TALENV0.2 RR right pCLS33934
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 216 - protein, 925 aa, synthetic construct
  REGION 1..925: note = CS1 TALEN V1 DD left pCLS34716
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 217 - protein, 925 aa, synthetic construct
  REGION 1..925: note = CS1 TALEN V1 DD right pCLS34717
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 218 - protein, 925 aa, synthetic construct
  REGION 1..925: note = CS1 TALEN V1.2 DD-RR left pCLS35196
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 219 - protein, 925 aa, synthetic construct
  REGION 1..925: note = CS1 TALEN V1.2 DD-RR right pCLS35197
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 220 - protein, 917 aa, synthetic construct
  REGION 1..917: note = TALE-BE V2 CD52-1 N
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 221 - protein, 839 aa, synthetic construct
  REGION 1..839: note = TALE-BE V2 CD52-1 C
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 222 - protein, 917 aa, synthetic construct
  REGION 1..917: note = TALE-BE V2 CD52-2 N
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 223 - protein, 839 aa, synthetic construct; identical to SEQ ID NO: 221
  REGION 1..839: note = TALE-BE V2 CD52-2 C

SEQ ID NO: 224 - protein, 917 aa, synthetic construct
  REGION 1..917: note = TALE-BE V2 CD52-3 N
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 225 - protein, 839 aa, synthetic construct; identical to SEQ ID NO: 221
  REGION 1..839: note = TALE-BE V2 CD52-3 C

SEQ ID NO: 226 - protein, 917 aa, synthetic construct
  REGION 1..917: note = TALE-BE V2 CD52-4 N
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 227 - protein, 839 aa, synthetic construct
  REGION 1..839: note = TALE-BE V2 CD52-4 C
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 228 - DNA, 49 bp, Homo sapiens
  misc_feature 1..49: note = CS1 target sequence
  source 1..49: mol_type = genomic DNA
ttccagagagcaatatggctggttccccaacatgcctcaccctcatcta

SEQ ID NO: 229 - DNA, 48 bp, Homo sapiens
  misc_feature 1..48: note = OS1 CS1 off site target sequence
  source 1..48: mol_type = genomic DNA
aagcagagaaggaagccctggaatgtgtagagagggcacccttatctt

SEQ ID NO: 230 - DNA, 52 bp, Homo sapiens
  misc_feature 1..52: note = OS2 CS1 off site target sequence
  source 1..52: mol_type = genomic DNA
ttccagagagccaaggaagctatttctatgactaattctctttcctcatcta

SEQ ID NO: 231 - DNA, 49 bp, Homo sapiens
  misc_feature 1..49: note = TALE V2 CTLA4 target sequence
  source 1..49: mol_type = genomic DNA
tcctagatgattccatctgcacgggcacctccagtggaaatcaagtgaa

SEQ ID NO: 232 - DNA, 49 bp, Homo sapiens
  misc_feature 1..49: note = TALE V2 CISH target sequence
  source 1..49: mol_type = genomic DNA
tgcgcctagtgacccagcactgcctgctcctccaccagccactgctgta

SEQ ID NO: 233 - DNA, 49 bp, Homo sapiens
  misc_feature 1..49: note = TALE V2 LAG3 target sequence
  source 1..49: mol_type = genomic DNA
tccaggatctcagccttctgcgaagagcaggggtcacttggcagcatca

SEQ ID NO: 234 - DNA, 49 bp, Homo sapiens
  misc_feature 1..49: note = TALE V2 TGFBRII target sequence
  source 1..49: mol_type = genomic DNA
tccctatgaggagtatgcctcttggaagacagagaaggacatcttctca

SEQ ID NO: 235 - DNA, 47 bp, Homo sapiens
  misc_feature 1..47: note = TALE V2 CCR5 target sequence
  source 1..47: mol_type = genomic DNA
tatcaagtgtcaagtccaatctatgacatcaattattatacatcgga

SEQ ID NO: 236 - DNA, 47 bp, Homo sapiens
  misc_feature 1..47: note = TALE V2 B2m target sequence
  source 1..47: mol_type = genomic DNA
ttagctgtgctcgcgctactctctctttctggcctggaggctatcca

SEQ ID NO: 237 - DNA, 47 bp, Homo sapiens
  misc_feature 1..47: note = TALE V2 B2m-2 target sequence
  source 1..47: mol_type = genomic DNA
tccgtggccttagctgtgctcgcgctactctctctttctggcctgga

SEQ ID NO: 238 - DNA, 48 bp, Homo sapiens
  misc_feature 1..48: note = TALE V2 TCR&#945; target sequence
  source 1..48: mol_type = genomic DNA
ttgtcccacagatatccagaaccctgaccctgccgtgtaccagctgag

SEQ ID NO: 239 - DNA, 49 bp, Homo sapiens
  misc_feature 1..49: note = TALE V2 PD1 target sequence
  source 1..49: mol_type = genomic DNA
tacctctgtggggccatctccctggcccccaaggcgcagatcaaagaga

SEQ ID NO: 240 - DNA, 47 bp, Homo sapiens
  misc_feature 1..47: note = TALE V2 PIK3CDex8 target sequence
  source 1..47: mol_type = genomic DNA
tgaacgccgacgagcggatgaaggtggggctcctgggataggtggga

SEQ ID NO: 241 - DNA, 49 bp, Homo sapiens
  misc_feature 1..49: note = TALE V2 PIK3CDex17 target sequence
  source 1..49: mol_type = genomic DNA
tgatgcacttgtgcatgcggcaggaggcctacctagaggccctctccca

SEQ ID NO: 242 - DNA, 49 bp, Homo sapiens
  misc_feature 1..49: note = TALE V2 S100A9 target sequence
  source 1..49: mol_type = genomic DNA
ttaggggccctgacagctctccataggtggaggcctcaggcaggcagga

SEQ ID NO: 243 - DNA, 46 bp, Homo sapiens
  misc_feature 1..46: note = TALE V2 AAVS1 target sequence
  source 1..46: mol_type = genomic DNA
tcccctccaccccacagtggggccactagggacaggattggtgaca

SEQ ID NO: 244 - DNA, 49 bp, Homo sapiens
  misc_feature 1..49: note = TALE V2 CD52 target sequence
  source 1..49: mol_type = genomic DNA
ttcctcctactcaccatcagcctcctggttatggtacaggtaagagcaa

SEQ ID NO: 245 - DNA, 49 bp, Homo sapiens
  misc_feature 1..49: note = TALE V2 TCR&#945;-2 target sequence
  source 1..49: mol_type = genomic DNA
tctgcctattcaccgattttgattctcaaacaaatgtgtcacaaagtaa

SEQ ID NO: 246 - DNA, 47 bp, Homo sapiens
  misc_feature 1..47: note = TALE V2 TGFBRII-2 target sequence
  source 1..47: mol_type = genomic DNA
tgtgtaaattttgtgatgtgagattttccacctgtgacaaccagaaa

SEQ ID NO: 247 - DNA, 48 bp, Homo sapiens
  misc_feature 1..48: note = TALE V2 TGFBRII-3 target sequence
  source 1..48: mol_type = genomic DNA
tgatgtgagattttccacctgtgacaaccagaaatcctgcatgagcaa

SEQ ID NO: 248 - DNA, 48 bp, Homo sapiens
  misc_feature 1..48: note = TALE V2 TGFBRII-4 target sequence
  source 1..48: mol_type = genomic DNA
tcgtcctgtggacgcgtatcgccagcacgatcccaccgcacgttcaga

SEQ ID NO: 249 - DNA, 48 bp, Homo sapiens
  misc_feature 1..48: note = TALE V2 CD52-1 target sequence
  source 1..48: mol_type = genomic DNA
ttttgtcctgagagtccagtttgtatctgtaggaggagaagtgggata

SEQ ID NO: 250 - DNA, 47 bp, Homo sapiens
  misc_feature 1..47: note = TALE V2 CD52-2 target sequence
  source 1..47: mol_type = genomic DNA
tttgtcctgagagtccagtttgtatctgtaggaggagaagtgggata

SEQ ID NO: 251 - DNA, 46 bp, Homo sapiens
  misc_feature 1..46: note = TALE V2 CD52-3 target sequence
  source 1..46: mol_type = genomic DNA
ttgtcctgagagtccagtttgtatctgtaggaggagaagtgggata

SEQ ID NO: 252 - DNA, 49 bp, Homo sapiens
  misc_feature 1..49: note = TALE V2 CD52-4 target sequence
  source 1..49: mol_type = genomic DNA
tggctggtgtcgttttgtcctgagagtccagtttgtatctgtaggagga

SEQ ID NO: 253 - DNA, 20 bp, synthetic construct
  misc_feature 1..20: note = TGFBRII F
  source 1..20: mol_type = other DNA
tcatcctggaagatgaccgc

SEQ ID NO: 254 - DNA, 20 bp, synthetic construct
  misc_feature 1..20: note = TGFBRII R
  source 1..20: mol_type = other DNA
tcatcctggaagatgaccgc

SEQ ID NO: 255 - DNA, 20 bp, synthetic construct
  misc_feature 1..20: note = OT1_F
  source 1..20: mol_type = other DNA
gagaaacctggcttgtagtg

SEQ ID NO: 256 - DNA, 20 bp, synthetic construct
  misc_feature 1..20: note = OT1_R
  source 1..20: mol_type = other DNA
catattcatgaaagggaagc

SEQ ID NO: 257 - DNA, 21 bp, synthetic construct
  misc_feature 1..21: note = OT2_F
  source 1..21: mol_type = other DNA
atcactcatggtctgcattag

SEQ ID NO: 258 - DNA, 20 bp, synthetic construct
  misc_feature 1..20: note = OT2_R
  source 1..20: mol_type = other DNA
ccagacctggaagagtagat

SEQ ID NO: 259 - DNA, 19 bp, synthetic construct
  misc_feature 1..19: note = OT3_F
  source 1..19: mol_type = other DNA
tgcgctcggctataacgat

SEQ ID NO: 260 - DNA, 19 bp, synthetic construct
  misc_feature 1..19: note = OT3_R
  source 1..19: mol_type = other DNA
tgaagtgacattcagacct

SEQ ID NO: 261 - DNA, 20 bp, synthetic construct
  misc_feature 1..20: note = OT4_F
  source 1..20: mol_type = other DNA
ggaattggcaactcatcagg

SEQ ID NO: 262 - DNA, 19 bp, synthetic construct
  misc_feature 1..19: note = OT4_R
  source 1..19: mol_type = other DNA
attgctaccgcacaactgg

SEQ ID NO: 263 - DNA, 20 bp, synthetic construct
  misc_feature 1..20: note = OT7_F
  source 1..20: mol_type = other DNA
gaagatctactctgaacctc

SEQ ID NO: 264 - DNA, 19 bp, synthetic construct
  misc_feature 1..19: note = OT7_R
  source 1..19: mol_type = other DNA
gtattagtcattctgcctg

SEQ ID NO: 265 - DNA, 21 bp, synthetic construct
  misc_feature 1..21: note = OT17_F
  source 1..21: mol_type = other DNA
gtgttatatacacacatggac

SEQ ID NO: 266 - DNA, 20 bp, synthetic construct
  misc_feature 1..20: note = OT17_R
  source 1..20: mol_type = other DNA
cttgcaaatatggtgcatcg

SEQ ID NO: 267 - protein, 925 aa, synthetic construct
  REGION 1..925: note = TGFBRII TALEN-classical (V0) left pCLS32967
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 268 - protein, 925 aa, synthetic construct
  REGION 1..925: note = TGFBRII TALEN-classical (V0) right pCLS32968
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 269 - protein, 925 aa, synthetic construct
  REGION 1..925: note = TIGIT left V2 TALEN pCLS39233
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 270 - protein, 925 aa, synthetic construct
  REGION 1..925: note = TIGIT right V2 TALEN pCLS39234
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 271 - protein, 925 aa, synthetic construct
  REGION 1..925: note = CISH (1) left TALEN V2 pCLS38090
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 272 - protein, 925 aa, synthetic construct
  REGION 1..925: note = CISH (1) right TALEN V2 pCLS38091
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 273 - protein, 925 aa, synthetic construct
  REGION 1..925: note = CISH (2) left TALEN V2 pCLS38094
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 274 - protein, 925 aa, synthetic construct
  REGION 1..925: note = CISH (2) right TALEN pCLS38095
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 275 - protein, 925 aa, synthetic construct
  REGION 1..925: note = CISH (3) left TALEN V2 pCLS38086
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 276 - protein, 925 aa, synthetic construct
  REGION 1..925: note = CISH (3) right TALEN V2 pCLS38087
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 277 - protein, 925 aa, synthetic construct
  REGION 1..925: note = CD38 (1) left TALEN V2 pCLS37948
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 278 - protein, 925 aa, synthetic construct
  REGION 1..925: note = CD38 (1) right TALEN V2 pCLS37949
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 279 - protein, 925 aa, synthetic construct
  REGION 1..925: note = CD38 (2) left TALEN V2 pCLS37931
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 280 - protein, 925 aa, synthetic construct
  REGION 1..925: note = CD38 (2) right TALEN V2 pCLS37932
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 281 - protein, 925 aa, synthetic construct
  REGION 1..925: note = IgH (1) left TALEN V2 pCLS39197
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 282 - protein, 925 aa, synthetic construct
  REGION 1..925: note = igH (1) right TALEN V2 pCLS39198
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 283 - protein, 925 aa, synthetic construct
  REGION 1..925: note = IgH (2) left TALEN V2 pCLS39194
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 284 - protein, 925 aa, synthetic construct
  REGION 1..925: note = igH (2) right TALEN V2 pCLS39195
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 285 - protein, 925 aa, synthetic construct
  REGION 1..925: note = GADPH (1) left TALEN V2 pCLS39327
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 286 - protein, 925 aa, synthetic construct
  REGION 1..925: note = GADPH (1) right TALEN V2 pCLS39329
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASHDGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 287 - protein, 925 aa, synthetic construct
  REGION 1..925: note = GADPH (2) left TALEN V2 pCLS39357
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 288 - protein, 925 aa, synthetic construct
  REGION 1..925: note = GADPH (2) right TALEN V2 pCLS39358
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 289 - DNA, 47 bp, synthetic construct
  misc_feature 1..47: note = TIGIT target sequence
  source 1..47: mol_type = other DNA
tgtcacctctcctccaccacggcacaagtgacccaggtcaactggga

SEQ ID NO: 290 - DNA, 49 bp, synthetic construct
  misc_feature 1..49: note = CISH (1) target sequence
  source 1..49: mol_type = other DNA
tcgaggaggtggcagagggtaccccagcccagacagagagtgagccaaa

SEQ ID NO: 291 - DNA, 49 bp, synthetic construct
  misc_feature 1..49: note = CISH (2) target sequence
  source 1..49: mol_type = other DNA
tggtattggggttccattacggccagcgaggcccgacaacacctgcaga

SEQ ID NO: 292 - DNA, 49 bp, synthetic construct
  misc_feature 1..49: note = CISH (3) target sequence
  source 1..49: mol_type = other DNA
tgcctgctggggccttcctcgaggaggtggcagagggtaccccagccca

SEQ ID NO: 293 - DNA, 47 bp, synthetic construct
  misc_feature 1..47: note = CD38 (1) target sequence
  source 1..47: mol_type = other DNA
tttcccgagaccgtcctggcgcgatgcgtcaagtacactgaaattca

SEQ ID NO: 294 - DNA, 47 bp, synthetic construct
  misc_feature 1..47: note = CD38 (2) target sequence
  source 1..47: mol_type = other DNA
tggagccctatggccaactgcgagttcagcccggtgtccggggacaa

SEQ ID NO: 295 - DNA, 47 bp, synthetic construct
  misc_feature 1..47: note = IgH (1) target sequence
  source 1..47: mol_type = other DNA
tgatatgtgtctggaattgaggccaaagcaagctcagctaagaaata

SEQ ID NO: 296 - DNA, 47 bp, synthetic construct
  misc_feature 1..47: note = IgH (2) target sequence
  source 1..47: mol_type = other DNA
tgagtgatatgtgtctggaattgaggccaaagcaagctcagctaaga

SEQ ID NO: 297 - DNA, 50 bp, synthetic construct
  misc_feature 1..50: note = GADPH (1) target sequence
  source 1..50: mol_type = other DNA
tcttattctagggtctggggcagaggggagggaagctgggcttgtgtcaa

SEQ ID NO: 298 - DNA, 47 bp, synthetic construct
  misc_feature 1..47: note = GADPH (2) target sequence
  source 1..47: mol_type = other DNA
tttgcttcccgctcagacgtcttgagtgctacaggaagctggcacca

(19) United States
(12) Patent Application Publication
(10) Pub. No.: US 2025/0002945 A1
(43) Pub. Date: Jan. 2, 2025

(54) NEW TALE PROTEIN SCAFFOLDS WITH IMPROVED ON-TARGET/OFF-TARGET ACTIVITY RATIOS

(71) Applicant: CELLECTIS SA, PARIS (FR)

(72) Inventors: Philippe DUCHATEAU, PARIS (FR); Alexandre JUILLERAT, New York, NY (US); Alex BOYNE, New York, NY (US); Selena KAZANCIOGLU, New York, NY (US)

(21) Appl. No.: 18/712,640

(22) PCT Filed: Nov. 23, 2022
(86) PCT No.: PCT/EP2022/082950

§ 371 Date: May 22, 2024

Related U.S. Application Data
(60) Provisional application No. 63/282,453, filed on Nov. 23, 2021.

(30) Foreign Application Priority Data
Mar. 15, 2022 (DK) PA202270104

(51) Int. Cl. C12N 15/90; C07K 14/195; C12N 9/22

(52) U.S. Cl. CPC C12N 15/907; C07K 14/195; C12N 9/22

(57) ABSTRACT

The present invention relates to the design of improved TALE protein fusions useful as sequence-specific genomic reagents, such as TALE-nucleases and TALE base editors, displaying higher on-target/off-target activity ratios. Its goal is to produce safer reagents to genetically modify the genomes of different types of cells, especially mammalian cells, in particular for their use in gene therapy.

### FIELD OF THE INVENTION

The present invention relates to the design of improved TALE protein fusions useful as sequence-specific genomic reagents displaying higher on-target/off-target activity ratios. Its goal is to produce safer reagents to genetically modify the genomes of different types of cells, especially mammalian cells, in particular for their use in gene therapy.

### SEQUENCE LISTING

The instant application contains a Sequence Listing which has been submitted electronically in XML file format and is hereby incorporated by reference in its entirety. Said XML copy, created on Jul. 8, 2024, is named DI2021-02US1_SL.xml and is 484,731 bytes in size.

### BACKGROUND OF THE INVENTION

Artificial transcription-activator-like effectors (TALE) form a special class of proteins that can bind DNA originally derived from the phytopathogenic bacterial genus Xanthomonas [Kay S. et al. (2007) A bacterial effector acts as a plant transcription factor and induces a cell size regulator. Science 318:648-651]. Artificial TALE proteins have emerged to be versatile and sequence specific gene tools offering flexible applications upon elucidation of a DNA recognition ‘code’, linking the amino-acid sequence of the TALE with its bound genomic DNA sequence [Moscou J.M. et al. (2009) A Simple Cipher Governs DNA Recognition by TAL Effectors. Science. 326:1501].

TALE binding is driven by a series of 33 to 35 amino-acid-long repeats that differ at essentially two positions, the so-called repeat variable dipeptide (RVD). Each base of one strand in the DNA target is contacted by a single repeat, with predictable specificity resulting from the linear arrangement of RVDs. The biochemical structure-function studies suggest that the amino acid present at position 13 uniquely identifies a nucleotide on the DNA target major groove [Deng D., et al. (2012) Structural basis for sequence-specific recognition of DNA by TAL effectors. Science 335:720-723; Stella S., et al. (2013) Structure of the AvrBs3-DNA complex provides new insights into the initial thymine-recognition mechanism. Acta Crystallogr Sect D Biol Crystallogr 69 (9): 1707-1716]. This DNA-protein interaction unit is stabilized by the amino acid at position 12. For the creation of TALEs with variable precision and binding affinity, six conventional RVDs are generally used (NG, HD, NI, NK, NH, and NN). HD and NG are associated with cytosine (C) and thymine (T) respectively. NN is a degenerate RVD showing binding affinity for both guanine (G) and adenine (A), but its specificity for guanine is reported to be stronger. RVD NI binds with A and NK binds with G. It is worth noting that the binding affinity of TALE is influenced by the methylation status of the target DNA sequence [Streubel J, et al. (2012) TAL effector RVD specificities and efficiencies. Nat Biotechnol 30 (7): 593-595.]. Methylated cytosine is not efficiently bound by the canonical RVDs. However, they can be accommodated by a certain degree of degeneracy in TALEs as described by Valton J, et al. [Overcoming transcription activator-like effector (TALE) DNA binding domain sensitivity to cytosine methylation (2012) J. Biol. Chem. 287 (46): 38427-38432]. This code was adopted to effectively engineer TALE DNA-binding scaffold specificity via modular assembly in order to form different associations of TALE proteins with various enzymatic domains, such as transcriptional activators, repressors, base editors or nucleases with potential ability to act on genomic sequences [Voytas et al. (2011) TAL effectors: Customizable proteins for DNA targeting. Science 333 (6051): 1843-6]. In comparison to Zinc-Finger protein fusions, TALE-proteins have significantly emerged as critical DNA-binding scaffolds governed by a simple cipher without significant restrictions. Their compatibility with a broad range of epigenetic modifiers is commendable [Laufer B.I., et al. (2015) Strategies for precision modulation of gene expression by epigenome editing: an overview. Epigenetics Chromatin 8 (1): 34.] and it is considered that, with these DNA-binding proteins, it is possible to target an epigenetic effector domain to any locus in the genome [Cano-Rodriguez D., Rots M.G. (2016) Epigenetic editing: on the verge of reprogramming gene expression at will. Curr Genet Med Rep 4 (4): 170-179.].

Such TALE protein fusions may result in TALE Artificial transcription factors, which have been generated by the fusion of TALE with a 16 amino acid peptide (VP16) from herpes simplex virus as a transactivation domain [Zhang, F. et al. Efficient construction of sequence-specific TAL effectors for modulating mammalian transcription. Nature Biotechnol. 29:149-153]. By contrast to zinc-fingers binding domains, which have encountered many off-target effects, TALE transcriptional activators are efficient transcription modulators with only 10.5 repeats with an effector module fused to the carboxyl terminal [Miller, J., et al. (2011) A TALE nuclease architecture for efficient genome editing. Nat Biotechnol. 29, 143-148]. TALEs in the form of activators can also be used to control the gene expression in case of external stimuli like a chemical change, or optical stimulus in various organisms including plants and animals.

TALE repressors can be generated by the fusion of TALE with either Kruppel-associated box (KRAB), Sid4, or EAR-repression domain (SRDX) repressors [Cong L, et al. (2012) Comprehensive interrogation of natural TALE DNA-binding modules and transcriptional repressor domains. Nat Commun 3 (1): 968].

TALE base editors can be generated by the fusion of TALE with deaminase, and sometimes, to other DNA repair proteins. Base editor catalytic domains can introduce single-nucleotide variants at desired loci in DNA (nuclear or organellar) or RNA of both dividing and non-dividing cells. Broadly, there are two types of DNA base editors that directly induce targeted point mutations in DNA, and RNA base editors that convert one ribonucleotide to another in RNA. Currently available DNA base editors can be further categorized into cytosine base editors (CBEs), adenine base editors (ABEs), C-to-G base editors (CGBEs), dual-base editors and organellar base editors. For instance, Mok et al. [A bacterial cytidine deaminase toxin enables CRISPR-free mitochondrial base editing (2020) Nature. 583:631-637] recently developed a base editing approach using the bacterial cytidine deaminase toxin, DddAtox, to demonstrate efficient C-to-T base conversions in vitro. In this approach, split DddAtox nontoxic halves fused to transcription activator-like effector (TALE) proteins, which can be custom-designed to recognize predetermined target DNA sequences, form a functional cytosine deaminase within the editing window to induce C-to-T base editing at the target site in genomic DNA. Such DddA-TALE fusion deaminase constructs have since achieved mitochondrial DNA editing in mice [Lee, H., et al. (2021) Mitochondrial DNA editing in mice with DddA-TALE fusion deaminases. Nat Commun 12: 1190].

TALE nucleases can be generated by the fusion of TALE with various nuclease catalytic domains. The popularly used TALEN® system, which provides specific nucleases as a fusion of TALE scaffolds with the catalytic domain of the Fok1 restriction enzyme has proven to be very specific through many studies, as it combines two TALE dimers that bind together at the selected locus. The TALEN heterodimers (right and left) generally bind on opposite strands at about 10-20 pb away from each other (spacer) to allow the nuclease Fok1 to dimerize and induce double strands cleavage between the binding sites within the spacer. This heterodimeric setting allows an increased sequence specificity based on the extended target sequence encompassed by the two TALE binding sites that can span up to 40 base pairs. Such TALE-nucleases are currently developed as therapeutic grade nuclease reagents in gene therapy, especially to produce allogeneic CAR-T cells [Poirot et al. (2015) Multiplex Genome-Edited T-cell Manufacturing Platform for “Off-the-Shelf” Adoptive T-cell Immunotherapies Cancer Res 75 (18): 3853-3864; Quasim W. et al. (2017) Molecular remission of infant B-ALL after infusion of universal TALEN gene-edited CAR T cells. Science translational medicine (9) 374]. The classical TALEN monomer construct is generally based on truncated version of the TALE binding domain from the AvrBs3 protein fused to the catalytic domain of Fok1, such as initially described by Voytas et al. in WO2011072246. Such TALE-nuclease fusion protein, referred to herein as “canonical”, typically comprises from 5′ to 3′: (1) truncated N-terminal region from AvrBs3 comprising at least the 150 amino acids that are proximal to the binding domain; (2) an engineered central DNA-binding domain which generally comprises between 12 to 28 repeats that are assembled to target a genomic nucleotide sequence; these selected repeats are followed by a wild type half repeat of only 20 amino acids from AvrBs3 designed to bind the 3′-end of the targeted DNA sequence; (3) a linker sequence of at least 40 amino acids from the C-terminal wild type region of AvrBs3 fused to (4) the wild type Fok1 nuclease catalytic domain. that In general the fusion protein further comprises AvrBs3's nuclear localization signal (NLS) fused to the truncated N-terminal region. Enhancements to the core TALE domain via various truncations have been proposed in several studies [Miller, J. C. et al. (2011) A TALE nuclease architecture for efficient genome editing. Nat. Biotechnol. 29, 143] along with the use of additional or alternative RVDs, which have shown to improve specificity and efficacy of these programmable TALE DNA-binding domain [Juillerat A, et al. (2015) Optimized tuning of TALEN specificity using non-conventional RVDs. Sci Rep 5 (1)].

Such bespoke TALE proteins have proven to be robust reagents for targeting genomic DNA sequences of interest in almost every cell types [Weeks D.P,. et al. Use of designer nucleases for targeted gene and genome editing in plants (2016) Plant Biotechnology Journal. 14:483-495; Mussolino C. et al. (2014) TALENs facilitate targeted genome editing in human cells with high specificity and low cytotoxicity. Nucleic Acids Res 42 (10): 6762-6773]. In addition, the TALE proteins engineered according to this standard scheme are very similar to each other in terms of structure and sequence identity. Indeed, only amino acids in positions 12 and 13 of each repeat in the central DNA binding domain need to differ to adapt the scaffold to new target sequences.

Nevertheless, with the development of TALE-nucleases for human gene therapy, standard TALE constructs do not always meet the specificity and efficiency levels required for therapeutic safety. Depending on the sequences to be targeted in the genome and their intrinsic variability in human populations, TALE scaffolds sometimes need further refinements to reduce potential off-target binding and increase their catalytic activity. Previous methods consisting in including additional or non-conventional RVDs may not be sufficient in all situations. In fact, specificity and catalytic activity are often in balance and it may be difficult to find a good compromise that preserves safety and efficiency.

To go beyond the current high standards of engineered TALE proteins, the inventors have designed new TALE scaffolds that combine different sets of mutations. The resulting TALE fusion proteins based on these new scaffolds show a better specificity, while retaining most of their catalytic activities, and remain adaptable to any target sequence and RVD adjustment. Their invention thus offers a platform for rational design of TALE catalytic proteins of higher therapeutic grade.

### SUMMARY OF THE INVENTION

The present invention aims at improving the specificity and/or activity of TALE fusion proteins which binding domain is generally based on the assembly of AvrBs3 repeats from original Xanthomonas genomic sequences.

As per the invention, the original AvrBs3 repeats of the TALE core binding domain have been fused with a C-terminal region consisting of a polypeptide sequence from 40 to 80 residues comprising a sequence having at least 85% identity with the following SEQ ID NO:2, SEQ ID NO: 3 or SEQ ID NO:4:

SEQ ID NO: 2 (C-40 AA):
SIVAQLSRPDPALAALTNDHLVALACLGGRPALDAVX₁X₂GL
SEQ ID NO: 3 (C-50 AA):
SIVAQLSRPDPALAALTNDHLVALACLGGRPALDAVX₁X₂GLPHAPALI
X₃RT
SEQ ID NO: 4 (C-60 AA):
SIVAQLSRPDPALAALTNDHLVALACLGGRPALDAVX₁X₂GLPHAPALI
X₃RTNRRIPERTH

wherein, X₁, X₂ and X₃ represent H (histidine) or R (arginine), preferably R. X₁, X₂, and X₃ can be identical or different.

In general, said TALE core binding domain is fused to a N-terminal region, which preferably comprises or consists of a polypeptide sequence showing at least 85%, preferably at least 90%, more preferably at least 95% identity with SEQ ID NO:1.

According to preferred embodiments, said TALE core binding domain comprises AvrBs3-like repeats, such as those comprising a D (aspartic acid) amino acid substitution at position 4 (D4) and/or at position 32 (D32) in their polypeptide sequence.

In some embodiments, said AvrBs3-like repeats comprise, or consist of, at least one of the following polypeptide sequences:

(SEQ ID NO: 5)
LTPDQVVAIASX₄X₅GGKQALETVQRLLPVLCQDHG,
(SEQ ID NO: 6)
LTPDQVVAIASX₄X₅GGKQALETVQALLPVLCQDHG
(SEQ ID NO: 7)
LTPDQVVAIASX₄X₅GGKQALETVQQLLPVLCQDHG,
(SEQ ID NO: 8)
LTPDQLVAIASX₄X₅GGKQALETVQRLLPVLCQDHG,
(SEQ ID NO: 9)
LTPDQMVAIASX₄X₅GGKQALETVQRLLPVLCQDHG,
(SEQ ID NO: 10)
LTPDQVVAIASX₄X₅GGKQALETVQRLLPVLCQDQG,
or
(SEQ ID NO: 11)
LTLDQVVAIASX₄X₅GGKQALETVQRLLPVLCQDHG,

wherein X₄X₅ are the di-residues interacting with a given nucleotide base pair in the targeted sequence. X₄ and X₅ can be any amino acid or null (referred to as * (star) to designate a missing residue in the RVD). X₄ and X₅ can be identical or different.

These selected sequences, in particular the combination thereof, have been found by the inventors to improve the overall TALE protein structure, leading to a tighter interaction with its target sequence reflecting more specificity. In the meantime, this structure remains flexible enough to maintain the activity of the catalytic domain fused to said binding domain to efficiently process DNA upstream or downstream of the binding site(s).

The present invention also encompasses methods for producing or expressing TALE fusion proteins, such as TALE-nucleases, TALE-base editors or TALE-transcriptional modulators in a cell for targeting a genomic sequence.

In particular, the present invention provides methods for designing a TALE protein for introducing a genetic modification into a polynucleotide sequence, said method comprising the steps of:
a) selecting a polynucleotide target sequence on which the genetic modification is intended;
b assembling polynucleotide sequences encoding AvrBs3-like repeat(s) to form a polynucleotide encoding a TALE-binding domain to bind said selected polynucleotide target sequence;
c) fusing to said polynucleotide encoding the TALE-binding domain at least:
   (1) a polynucleotide sequence encoding a N-terminal domain comprising a sequence having at least 85% identity with SEQ ID NO:1, and
   (2) a polynucleotide sequence encoding a C-terminal domain consisting of a polypeptide sequence from 40 to 80 residues comprising a sequence having at least 85%, preferably 90%, more preferably 95% and even more preferably 99% identity with SEQ ID NO:2, SEQ ID NO:3 or SEQ ID NO:4; X₁, X₂, X₃ in these sequences representing R (arginine) or H (histidine); and optionally,
d) fusing a polynucleotide sequence encoding a catalytic domain, such as a nuclease or a deaminase to the polynucleotide sequence encoding said C-terminal domain;
e) fusing to the polynucleotide sequence encoding said N-terminal domain, a polynucleotide encoding a NLS (Nuclear Localization Signal), such as one listed in Table 1.

The methods of the invention aim to produce polynucleotides encoding TALE fusion proteins, as well as the polypeptides resulting from their expression.

The TALE proteins according to the present invention generally display improved on-target/off-target activity ratios with respect to the targeted genomic sequence compared to TALE fusion proteins of the prior art

The method of the invention can further include steps wherein the new polynucleotide sequences are expressed in cells to obtain, for instance, cleavage, base substitution or transcriptional activation at a targeted genomic locus and compare its efficiency with other TALE proteins to select one with higher on-target/off-target activity ratio.

The method of the invention can also include steps, wherein at least one of said AvrBs3-like repeats is further mutated in 1, 2, 3 and up to 5 amino acid positions in addition to the D4 and D32 substitutions.

The method of the invention can also include steps, wherein the C-terminal domain of the TALE protein is mutated to introduce 1 to 5 positively charged amino acids, such as lysine (K), arginine (R) or histidine (H), in addition to said X₁, X₂, and X₃ positions referred to previously.

The method of the invention can also include an additional step, wherein amino acid substitutions are introduced in the catalytic domain of the TALE protein to enhance its catalytic activity.

In further embodiments, the invention is drawn to recombinant transcriptional activator-like Effector (TALE) proteins comprising one or several AvrBs3-like repeats, comprising generally from 8 to 20 repeats, preferably from 8 to 18, more preferably from 10 to 16, and alternatively from 5 to 12 repeats in situations where smaller genomes are considered, such as for instance mitochondrial genomes.

In some embodiments, TALE proteins according to the present invention combine RVD repeats preferably AvrBs3-like repeats comprising the above amino acid substitutions, along with a C-terminal sequence, such as SEQ ID NO:2, SEQ ID NO:3 or SEQ ID NO:4, and a N-terminal sequence comprising SEQ ID NO: 1.

The recombinant core TALE proteins of the present invention are intended to be fused to a variety of catalytic domains as already described in the prior art (see WO2012138939), in particular catalytic domains from nucleases, such as Fok1 or Tev1, deaminases, such as cytidine deaminase toxin, and transcriptional modulators, such as the trans-activator VP16.

In some instances, the TALE protein of the invention is a TALE-nuclease that comprises a polypeptide sequence showing at least 85% identity, preferably at least 90%, more preferably at least 95%, even more preferably 99% identity with SEQ ID NO: 109, said polypeptide sequence corresponding to the catalytic domain of Fok-1 into which amino acid substitutions have been introduced to enhance the cleavage activity of the TALE-nuclease and improve its specificity.

The present application discloses many examples of TALE proteins produced according to the principles of the present invention, also referred to as “TALE V2”, in particular TALE-Base editors and TALE-nucleases, directed to a gene locus selected from TCRalpha, B2m, PD1, CTLA4, CISH, LAG3, TGFBRII, TIGIT, CD38, IgH, GADPH S100A9, PIK3CD, AAVS1 and CCR5, such as those listed in Tables 4 and 5.

The invention encompasses vectors comprising the polynucleotide sequences as well as the polypeptide sequences or reagents obtainable by the present invention, as well as their use for cell transformation and gene modification.

### DESCRIPTION OF FIGURES AND TABLES

FIG. 1: Structure of an illustrative TALE-nuclease protein fusion as per the present invention.

FIG. 2: Diagram comparing % indels (cleavage activity) obtained with V0, V0.1 and V0.2 TALE protein structures detailed in the examples.

FIG. 3: Diagram comparing overall off-site cleavage as resulting from oligo capture analysis (OCA) obtained with V0 and V0.1 TALE protein structures.

FIG. 4: Diagrams comparing indels formation of V1 and V1.2 TALE proteins according to the invention with the canonical TALE structure V0. A: % indels relative to V0 (cleavage activity at CS1 traget site is maintained), B: % indels observed at off-site locus OS1; C: % indels observed at off-site locus OS2 (V1 and V1.2 TALE structures abolish off-site cleavage).

FIG. 5: Diagram showing the reduction of overall off-site cleavage using V1 and V1.2 TALE-protein structures according to the present invention (Oligo capture assay) as detailed in the examples.

FIG. 6: Diagrams showing % indels obtained on-site (CS1 target sequence), and off-site (OS1 and OS2 loci) when alanine substitutions are introduced into the amino acid sequence of Fok1 (relative to wild type Fok1) at the position indicated in X axis.

FIG. 7: Diagram showing on-site indels compared to WT Fok1 (black bars) and off-site indels fold decrease compared to WT and observed at OS1 (white bars) when using TALE-nuclease with best substituted positions introduced in the Fok1 catalytic domain.

FIG. 8: Schematic representation of a TALE-base editor scaffold according to the present invention to inactivate the CD52 gene as described in Example 5.

FIG. 9: Histogram comparing % indels (cleavage activity) obtained with a TALE-nuclease targeting TGFBRII with either V0-V0, V1.2-V0, or V1.2-V1.2 heterodimeric structures at the on-target (on-site) or off-target sites (OT #). V1.2 comprises the TALE structure according to the present invention as detailed in Example 6.

FIG. 10: diagrams showing the results of the Oligo Capture Assays (OCA) performed on the cells transfected with the TALE-nucleases V2 designed according to the present invention to target TIGIT.

FIG. 11: diagrams showing the results of the Oligo Capture Assays (OCA) performed on the cells transfected with the TALE-nucleases V2 designed according to the present invention to target CISH (against three different target sequences 1, 2 and 3).

FIG. 12: diagrams showing the results of the Oligo Capture Assays (OCA) performed on the cells transfected with the TALE-nucleases V2 designed according to the present invention to target CD38 (against two different target sequences 1 and 2).

FIG. 13: diagrams showing the results of the Oligo Capture Assays (OCA) performed on the cells transfected with the TALE-nucleases V2 designed according to the present invention to target IgH (against two different target sequences 1 and 2).

FIG. 14: diagrams showing the results of the Oligo Capture Assays (OCA) performed on the cells transfected with the TALE-nucleases V2 designed according to the present invention to target GAPDH (against two different target sequences 1 and 2).

FIG. 15: percentage of Indels measured on the cells transfected with the respective TALE-nucleases V2 according to the present invention that are presented in Example 7.

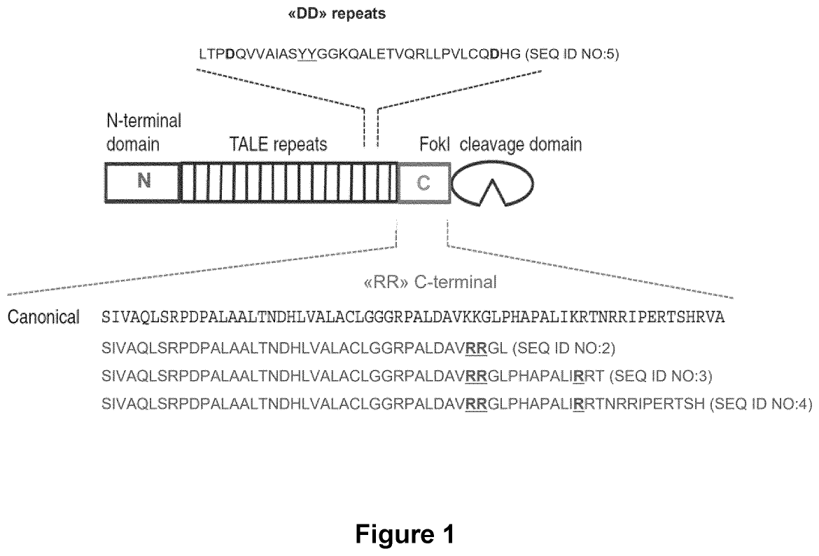

Table 1: Example of NLS polypeptide sequences

Table 2: Example of linkers that may be included in the TALE fusion proteins.

Table 3: Example of catalytic domains

Table 4: Examples of TALE proteins according to the present invention useful in gene therapy or adoptive immune cells therapy

Table 5: Polypeptide sequences used in the examples.

Table 6: Polynucleotide sequences used in the examples.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology [Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press; Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986].

The present invention has thus for object methods to design and produce TALE proteins that display reduced off-target DNA binding, which can be fused to various catalytic domains in view of forming highly specific and active TALE fusion proteins, in particular TALE-nucleases and TALE-base editors.

According to some embodiments, the invention provides methods for designing a TALE protein for introducing a genetic modification into a polynucleotide sequence, said method comprising one or several of the following steps:
a) selecting a polynucleotide target sequence on which the genetic modification is intended;
b) assembling polynucleotide sequences encoding AvrBs3-like repeat(s) to form a polynucleotide encoding a TALE-binding domain to bind said selected polynucleotide target sequence;
c) fusing to said polynucleotide encoding the TALE-binding domain at least:
   (1) a polynucleotide sequence encoding a N-terminal domain comprising a sequence having at least 85% identity with SEQ ID NO: 1, and
   (2) a polynucleotide sequence encoding a C-terminal domain consisting of a polypeptide sequence from 40 to 80 residues comprising a sequence having at least 85%, preferably 90%, more preferably 95% and even more preferably 99% identity with SEQ ID NO:2, SEQ ID NO:3 or SEQ ID NO:4; X₁, X₂, X₃ in these sequences representing R (arginine) or H (histidine); and optionally,

In general, the above steps can be performed in-silico and the final polynucleotide sequence synthetised or cloned according to methods well known in the art, such as explained for instance in WO2013017950.

By <<genetic modification>> is intended any enzymatic reaction voluntarily induced at a given locus, such as a mutation, methylation, transcriptional modulation, in view of obtaining an effect on gene expression.

According to some embodiments, the methods of the invention comprise one or several of the steps consisting of:
a) selecting a cleavage site in a target polynucleotide sequence, such as into a genome, where cleavage is intended;
b) selecting a polynucleotide sequence located between 5 and 25 bp upstream and/or downstream of said cleavage site;
c) assembling polynucleotide sequences encoding AvrBs3-like repeat(s) to encode a TALE-binding domain to bind said selected polynucleotide sequence, wherein at least one AvrBs3-like repeat(s) comprises D substitutions at positions 4 (D4) and 32 (D32) in its polypeptide sequence, such as one sequence selected from SEQ ID NO:5 to 11;
d) fusing said TALE-binding domain to at least (1) a polynucleotide sequence encoding a N-terminal domain, preferably comprising a sequence having at least 85%, preferably at least 90%, more preferably at least 95% identity with SEQ ID NO: 1 and
   (2) a polynucleotide sequence encoding a C-terminal domain preferably of a polypeptide sequence from 40 to 80 residues comprising a sequence having at least 85% identity with SEQ ID NO:2, SEQ ID NO:3 or SEQ ID NO:4, (X₁, X₂, X₃ in these sequences representing R (arginine) or H (histidine));
e) fusing the polynucleotide sequence obtained in d) with another polynucleotide sequence encoding a nuclease, such as a type II endonuclease, in particular Fok1.

The present method can also comprise optional steps, wherein, for instance, the polynucleotide sequence that is fused to the TALE protein and encode the catalytic domain can be mutated to introduce amino acid substitutions into said catalytic domain. This approach is exemplified in the experimental part of the present application, where amino acids have been substituted by alanine residues in the Fok1 catalytic domain (SEQ ID NO: 109) with the effect of obtaining an optimal nuclease activity of a TALE-nuclease according to the invention. Such individual substitutions in the Fok1 catalytic domain that have been found to decrease off-site activity are particularly those at positions 13, 52, 57, 59, 61, 65, 84, 85, 88, 91, 92, 95, 98, 103, 109, 110, 111, 113, 119, 143, 148, 152, 158, 159, 160, 167, 169, 170 and 194 into SEQ ID NO: 109. Preferred substitutions are at positions 84, 85, 88, 95, 98, 91, 103, 109, 148, 152 and 158, and most preferred ones are in positions 84, 88, 91, 103 and 152 into SEQ ID NO: 109.

By “TALE protein”, is meant herein a polypeptide that typically comprises a core DNA binding domain, which has at least 50%, preferably at least 60%, 70%, 80% or 90% identity with the DNA binding domain of wild-type AvrBs3 [also called TalC Uniprot-G7TLQ9], which represents the archetype of the family of transcription activator-like (TAL) effectors from phytopathogenic Xanthomonas campestris. Such DNA binding domain is characterized by repeated sequences of about 30 and 34 amino acids comprising variable di-residues usually found in positions 12 and 13. A consensus sequence for these repeats, also called RVDs, has been established for each targeted base A, C, G and T, which are respectively:

(SEQ ID NO: 31)
LTPQQVVAIASNIGGKQALETVQRLLPVLCQQHG for targeting
A;
(SEQ ID NO: 32)
LTPQQVVAIASHDGGKQALETVQRLLPVLCQQHG for targeting
C;
(SEQ ID NO: 33)
LTPQQVVAIASNNGGKQALETVQRLLPVLCQQHG for targeting
G;
(SEQ ID NO: 34)
LTPQQVVAIASNGGGKQALETVQRLLPVLCQQHG for targeting
T.

By “AvrBs3-like repeats” are meant artificial arrays of about 30 to 33 amino acids, which typically comprise variable di-residues in positions 12 and 13 interacting with A, C, G or T, similarly as the above consensus AvrBs3 repeats. In other words, AvrBs3-like repeats are similar and can be combined with AvrBs3 repeats, but are generally not identical to the consensus or to the wild-type AvrBs3 repeats. It shall be noted that, in some instances, di-residues in positions 12 or 13 may be absent-so-called * (star)—to accommodate methylated bases in genomic DNA as described by [Valton et al. (2012) Overcoming Transcription Activator-like Effector (TALE) DNA Binding Domain Sensitivity to Cytosine Methylation. DNA and Chromosomes. 287 (46): 38427].

The AvrBs3-like repeats of the present invention generally display at least 60%, preferably at least 70%, 75%, 80%, 90% or 95% identity with either of the above AvrBs3 consensus repeats sequences of SEQ ID NO:31 to 34. They generally comprise D4 and D32 substitutions, such as in the following repeat sequences SEQ ID NO:5 to 11 of the present invention:

(SEQ ID NO: 5)
LTPDQVVAIASX₄X₅GGKQALETVQRLLPVLCQDHG,
(SEQ ID NO: 6)
LTPDQVVAIASX₄X₅GGKQALETVQALLPVLCQDHG
(SEQ ID NO: 7)
LTPDQVVAIASX₄X₅GGKQALETVQQLLPVLCQDHG,
(SEQ ID NO: 8)
LTPDQLVAIASX₄X₅GGKQALETVQRLLPVLCQDHG,
(SEQ ID NO: 9)
LTPDQMVAIASX₄X₅GGKQALETVQRLLPVLCQDHG,
(SEQ ID NO: 10)
LTPDQVVAIASX₄X₅GGKQALETVQRLLPVLCQDQG,
or
(SEQ ID NO: 11)
LTLDQVVAIASX₄X₅GGKQALETVQRLLPVLCQDHG,

wherein X₄X₅ are the di-residues interacting with a given nucleotide base pair in the targeted sequence. X₄ and X₅ can be any amino acid or null (referred to as * (star) to designate a missing residue in the RVD). X₄ and X₅ can be identical or different.

The AvrBs3-like repeats are generally represented by polypeptide sequences, in which X₄ and X₅ are respectively NI (to preferably target A), HD (to preferably target C), (to preferably target G) NN and NG (to preferably target T), such as in SEQ ID NO:24, 25, 26 and 27.

“Identity” throughout the present specification refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. The present specification generally encompasses polypeptides and polynucleotides having at least 70%, 85%, 90%, 95%, 98% or 99% identity with the specific polypeptides and polynucleotides sequences described herein, exhibiting substantially the same functions or that can be considered as equivalents.

In some embodiments, the invention also provides a recombinant transcriptional activator-like Effector (TALE) protein comprising one or several AvrBs3-like repeats comprising D (aspartic acid) residues at positions 4 and 32, such as in the above polynucleotide sequences SEQ ID NO: 5 to 11. Such AvrBs3-like repeats can be further mutated into 1 to 5 amino acid positions, including or in addition to the D4 and D32 positions. Such recombinant transcriptional activator-like Effector (TALE) proteins can comprise one or several of such repeats, to form polypeptides comprising generally from 8 to 20 repeats, preferably from 8 to 18, more preferably from 10 to 16, and alternatively from 5 to 12 repeats in situations where smaller genomes are considered, such as for instance mitochondrial genomes.

The variable di-residues (X₄X₅) present in the AvrBs3-like repeats and associated with recognition of the different nucleotides are generally HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. More preferably, RVDs associated with recognition of the nucleotides C, T, A, G/A and G respectively are selected from the group consisting of NN or NK for recognizing G, HD for recognizing C, NG for recognizing T and NI for recognizing A, TL for recognizing A, VT for recognizing A or G and SW for recognizing A. More generally, RVDs associated with recognition of nucleotide C are selected from the group consisting of N+, RVDs associated with recognition of the nucleotide T are selected from the group consisting of N* and H*, where * may denote a gap in the repeat sequence that corresponds to a lack of amino acid residue at the second position of the RVD. In some embodiments, X₄X₅ can represent unusual or unconventional amino acid residues in order to modulate their specificity towards nucleotides A, T, C and G as described in Juillerat et al. [Optimized tuning of TALEN specificity using non-conventional RVDs (2015) Sci Rep 5:8150].

Although not mandatory, the core DNA binding domain generally comprises a half RVD made of 20 amino acids located at the C-terminus. Said core DNA binding domain thus comprises between 8.5 and 30.5 RVDs, more preferably between 8.5 and 20.5 RVDs, and even more preferably, between 10,5 and 15.5 RVDs.

As per the present invention, the core DNA binding domain as previously described, preferably comprising RVDs bearing D4 and/or D32 substitutions, is flanked by N-terminal and C-terminal sequences, said N-terminal and C-terminal sequences having preferably one of the following features detailed below.

In some embodiments, the N-terminal sequence is derived from the N-terminal domain of a naturally occurring TAL effector such as AvrBs3. In another embodiment, said additional N-terminus domain is the full-length N-terminus domain of a naturally occurring TAL effector N-terminus domain. In a further embodiment, said additional N-terminus domain is a variant which allows overcoming sequence constraints associated with the so-called “RVDO” (i.e. first cryptic repeat), such as for instance the necessity to have a T required as the first base on the binding nucleic acid sequence.

In another embodiment, said N-terminal sequence is derived from a naturally occurring TAL effector or a variant thereof. In another embodiment, said N-terminal sequence is a truncated N-terminus of such naturally occurring TAL effector or variant. In another embodiment, said additional domain is a truncated version of AvrBs3 TAL effector. In another embodiment, said truncated version lacks its N-terminal segment distal from the core TALEbinding domain, such as the first 152 N-terminal amino acids residues of the wild type AvrBs3, or at least the 152 amino acids residues.

In some preferred embodiments, said N-terminal sequence comprises a polypeptide sequence showing at least 85%, preferably at least 90%, more preferably at least 95% identity with SEQ ID NO: 1.

In some embodiments, the C-terminal sequence corresponds to a full or preferably truncated C-terminal region of a naturally occurring TAL effector such as AvrBs3. In general, said C-terminal sequence is a truncated version of AvrBs3 TAL effector, proximal to the core TALE binding domain, such as SEQ ID NO:28 (40 amino acids), SEQ ID NO:29 (50 amino acids) or SEQ ID NO: 30 (60 amino acids) or a natural variant thereof. Accordingly, said C-terminal sequence generally comprises or consists of a polypeptide sequence from 40 to 80 residues comprising a sequence having at least 85% identity with the below SEQ ID NO:2, SEQ ID NO:3 or SEQ ID NO: 4:

SEQ ID NO: 2 (C-40 AA):
SIVAQLSRPDPALAALTNDHLVALACLGGRPALDAVX₁X₂GL
SEQ ID NO: 3 (C-50 AA):
SIVAQLSRPDPALAALTNDHLVALACLGGRPALDAVX₁X₂GL
PHAPALIX₃RT
SEQ ID NO: 4 (C-60 AA):
SIVAQLSRPDPALAALTNDHLVALACLGGRPALDAVX₁X₂GL
PHAPALIX₃RTNRRIPERTH

In the above sequences, X₁, X₂ and X₃ represent an amino acid substitution introduced into the wild type AvrBs3 C-terminal polypeptide sequence, which is preferably R (arginine) or H (histidine) residue, most preferably R, instead of originally K. X₁, X₂ and X₃ can be identical or different.

Said N-terminal sequence or C-terminal sequence can comprise a localization sequence (or signal) which allows targeting said chimeric protein toward a given organelle within an organism, a tissue or a cell. Non-limiting examples of such localization signals are nuclear localization signals, chloroplastic localization signals or mitochondrial localization signals. In another embodiment, said additional N-terminus domain can comprise a nuclear export signal having the opposite effect of a nuclear localization signal to help targeting organelles such as chloroplasts or mitochondria. In the scope of the present invention are also encompassed additional C-terminus or N-terminus sequences with a combination of several localization signals. Such combinations can be as a non-limiting example a nuclear localization signal (NLS) and/or a tissue-specific signal to help addressing said fusion protein of the present invention in the nuclear of tissue specific cells. In preferred embodiments, a NLS is generally included in the N-terminal region of the TALE-protein. A preferred NLS sequence comprises the polypeptide sequence SEQ ID NO: 12 derived from SV40, SEQ ID NO: 13 derived from C-Myc or SEQ ID NO: 14 derived from nucleoplasmin.

TABLE 1
Examples of NLS sequences

| SEQ | Original | |
|---|---|---|
| ID # | sequence | Polypeptide sequence |
| 12 | NLS SV40 | PKKKRKV |
| 13 | C-Myc NLS | PAAKKKKLD |
| 14 | Nucleoplasmin | KRPAATKKAGQAKKKK |
| | NLS | |
| 15 | VACM-1/CUL5 NLS | PKLKRQ |
| 16 | CXCR4 NLS | RPRK |
| 17 | VP1 NLS | RRARRPRG |
| 18 | 58BP1 NLS | GKRKLITSEEERSPAKRGRKS |
| 19 | ING4 NLS | KGKKGRTQKEKKAARARSKGKN |
| 20 | IER5 NLS | RKRCAAGVGGGPAGCPAPGSTPLKKPRR |
| 21 | ERK5 NLS | RKPVTAQERQREREEKRRRRQERAKEREK |
| | | RRQERER |
| 22 | cytochrome c | SVLTPLLLRGLTGSARRLPVPRAKIHSL |
| | oxidase | |
| | subunit 8A | |
| | mitochondrial | |
| | addressing | |
| | signals | |
| 23 | superoxide | LSRAVCGTSRQLAPVLGYLGSRQKHSLPD |
| | dismutase | |
| | 2 mitochondrial | |
| | addressing | |
| | signals | |

By “TALE fusion protein” is meant a TALE-protein which is linked to a polypeptide domain that confers a catalytic activity to said TALE protein. A TALE fusion protein can be for instance a sequence-specific reagent that processes DNA at the locus specified by the TALE binding domain. The fusion with the TALE protein can be made with the catalytic domain from an existing protein, such as a DNA processing enzyme, especially one having an activity selected from the group consisting of nuclease activity, polymerase activity, deaminase activity, kinase activity, phosphatase activity, methylase activity, topoisomerase activity, integrase activity, transposase activity, ligase activity, helicase activity, reverse transcriptase and recombinase activity.

In some embodiments, the TALE fusion protein according to the present invention can comprise a peptide linker to fuse the catalytic domain to said previously described core scaffold, or more preferably to link the C-terminal or N-terminal of said TALE protein to said catalytic domain. Such linker is generally flexible. Such as one linker sequence selected from the group consisting of NFS1, NFS2, CFS1, RM2, BQY, QGPSG, LGPDGRKA, 1a8h_1, 1dnpA_1, 1d8cA_2, 1ckqA_3, 1sbp_1, 1ev7A_1, 1alo_3, 1amf_1, 1adjA_3, 1fcdC_1, 1al3_2, 1g3p_1, 1acc_3, 1ahjB_1, 1acc_1, 1af7_1, 1heiA_1, 1bia_2, 1igtB_1, 1nfkA_1, 1au7A_1, 1bpoB_1, 1b0 pA_2, 1c05A_2, 1gcb_1, 1bt3A_1, 1b30B_2, 16vpA_6, 1dhx_1, 1b8aA_1, 1qu6A_1 optionally comprising SGGSGS stretches at either or both N and C-terminal ends that surround a variable region of 3 to 28 amino acids as exemplified in Table 2 below (SEQ ID NO:35 to 108).

TABLE 2
Example of peptide linkers.

| | | | SEQ |
|---|---|---|---|
| Name of the | | | ID |
| linker | Length | Sequence | NO: # |
| 1a8h_1 | 3 | NVG | NA |
| 1dnpA_1 | 4 | DSVI | 35 |
| 1d8cA_2 | 4 | IVEA | 36 |
| 1ckqA_3 | 4 | LEGS | 37 |
| 1sbp_1 | 4 | YTST | 38 |
| 1ev7A_1 | 5 | LQENL | 39 |
| 1alo_3 | 5 | VGRQP | 40 |
| 1amf_1 | 5 | LGNSL | 41 |
| 1adjA_3 | 6 | LPEEKG | 42 |
| 1fcdC_1 | 6 | QTYQPA | 43 |
| 1al3_2 | 6 | FSHSTT | 44 |
| 1g3p_1 | 7 | GYTYINP | 45 |
| 1acc_3 | 7 | LTKYKSS | 46 |
| 1ahjB_1 | 8 | SRPSESEG | 47 |
| 1acc_1 | 8 | PELKQKSS | 48 |
| 1af7_1 | 8 | LTTNLTAF | 49 |
| 1heiA_1 | 9 | TATPPGSVT | 50 |
| 1bia_2 | 9 | LDNFINRPV | 51 |
| 1igtB_1 | 9 | VSSAKTTAP | 52 |
| 1nfkA_1 | 10 | DSKAPNASNL | 53 |
| 1au7A_1 | 10 | KRRTTISIAA | 54 |
| 1bpoB_1 | 11 | PVKMFDRHSSL | 55 |
| 1b0pA_2 | 11 | APAETKAEPMT | 56 |
| 1c05A_2 | 14 | YTRLPERSELPAEI | 57 |
| 1gcb_1 | 14 | VSTDSTPVTNQKSS | 58 |
| 1bt3A_1 | 14 | YKLPAVTTMKVRPA | 59 |
| 1b3B_2 | 15 | IARTDLKKNRDYPLA | 60 |
| 16vpA_6 | 21 | TEEPGAPLTTPPTLHGNQARA | 61 |
| 1dhx_1 | 21 | ARFTLAVGDNRVLDMASTYFD | 62 |
| 1b8aA_1 | 26 | IVVLNRAETPLPLDPTGKVKAELDTR | 63 |
| 1qu6A_1 | 28 | ILNKEKKAVSPLLLTTTNSSEGLSMGNY | 64 |
| NFS1 | 20 | GSDITKSKISEKMKGQGPSG | 65 |
| NFS2 | 23 | GSDITKSKISEKMKGLGPDGRKA | 66 |
| CFS1 | 10 | SLTKSKISGS | 67 |
| RM2 | 32 | AAGGSALTAGALSLTAGALSLTAGALSGGGGS | 68 |
| BQY | 27 | AAGASSVSASGHIAPLSLPSSPPSVGS | 69 |
| QGPSG | 5 | QGPSG | 70 |
| LGPDGRKA | 8 | LGPDGRKA | 71 |
| TAL1 | 15 | SGGSGSNVGSGSGSG | 72 |
| TAL2 | 20 | SGGSGSLTTNLTAFSGSGSG | 73 |
| TAL3 | 22 | SGGSGSKRRTTISIAASGSGSG | 74 |
| TAL4 | 17 | SGGSGSVGRQPSGSGSG | 75 |
| TAL5 | 26 | SGGSGSYTRLPERSELPAEISGSGSG | 76 |
| TAL6 | 38 | SGGSGSIVVLNRAETPLPLDPTGKVKAELDTRSGSGSG | 77 |
| TAL7 | 21 | SGGSGSTATPPGSVTSGSGSG | 78 |
| TAL8 | 21 | SGGSGSLDNFINRPVSGSGSG | 79 |
| TAL9 | 21 | SGGSGSVSSAKTTAPSGSGSG | 80 |
| TAL10 | 22 | SGGSGSDSKAPNASNLSGSGSG | 81 |
| TAL11 | 23 | SGGSGSPVKMFDRHSSLSGSGSG | 82 |
| TAL12 | 23 | SGGSGSAPAETKAEPMTSGSGSG | 83 |
| TAL13 | 26 | SGGSGSVSTDSTPVTNQKSSSGSGSG | 84 |
| TAL14 | 16 | SGGSGSDSVISGSGSG | 85 |
| TAL15 | 33 | SGGSGSARFTLAVGDNRVLDMASTYFDSGSGSG | 86 |
| TAL16 | 17 | SGGSGSLQENLSGSGSG | 87 |
| TAL17 | 19 | SGGSGSGYTYINPSGSGSG | 88 |
| TAL18 | 26 | SGGSGSYKLPAVTTMKVRPASGSGSG | 89 |
| TAL19 | 16 | SGGSGSLEGSSGSGSG | 90 |
| TAL20 | 16 | SGGSGSIVEASGSGSG | 91 |
| TAL21 | 18 | SGGSGSQTYQPASGSGSG | 92 |
| TAL22 | 27 | SGGSGSIARTDLKKNRDYPLASGSGSG | 93 |
| TAL23 | 18 | SGGSGSLPEEKGSGSGSG | 94 |
| TAL24 | 16 | SGGSGSYTSTSGSGSG | 95 |
| TAL25 | 20 | SGGSGSSRPSESEGSGSGSG | 96 |
| TAL26 | 17 | SGGSGSLGNSLSGSGSG | 97 |
| TAL27 | 19 | SGGSGSLTKYKSSSGSGSG | 98 |
| TAL28 | 33 | SGGSGSTEEPGAPLTTPPTLHGNQARASGSGSG | 99 |
| TAL29 | 18 | SGGSGSFSHSTTSGSGSG | 100 |
| TAL30 | 20 | SGGSGSPELKQKSSSGSGSG | 101 |
| TAL31 | 40 | SGGSGSILNKEKKAVSPLLLTTTNSSEGLSMGNYSGSGSG | 102 |
| TAL32 | 31 | ELAEFHARYADLLLRDLRERPVSLVRGPDSG | 103 |
| TAL33 | 31 | ELAEFHARPDPLLLRDLRERPVSLVRGLGSG | 104 |
| TAL34 | 26 | ELAEFHARYADLLLRDLRERSGSGSG | 105 |
| TAL35 | 31 | DIFDYYAGVAEVMLGHIAGRPATRKRWPNSG | 106 |
| TAL36 | 31 | DIFDYYAGPDPVMLGHIAGRPATRKRWLGSG | 107 |
| TAL37 | 26 | DIFDYYAGVAEVMLGHIAGRSGSGSG | 108 |

In some embodiments, said peptide linker can comprise a calmodulin domain that changes TALE fusion protein conformation under calcium stimulation. Other protein domains inducing conformational changes under a specific metabolite interaction can also be used. Such linker can comprise, for instance, a light sensitive domain that allows a change from a folded inactive state toward an unfolded active state under light stimulation, or reverse. Other examples of “switch” linkers can be reactive to small molecules such as Chemical Inducers of Dimerization (CID).

In preferred embodiments, as illustrated herein with the preferred C-terminal sequences previously described, a linker may not be necessary to fuse the TALE core binding domain with the catalytic domain, as the C-terminal sequences can have enough flexibility to achieve an optimal conformation of the TALE fusion protein.

The present invention encompasses TALE fusion proteins comprising a variety of functional domains, such as catalytic domains obtainable from different enzymes. Such catalytic domains can be unspecific endonucleases such as for instance Fok-1, clo51 or I-Tev1, or specific endonuclease, such as engineered meganucleases (e.g. derived from I-Cre1, I-Onu1, I-Bmo1, Hmul . . . ), exonucleases such as human Trex2, transcription repressors (e.g. KRAB) or transcription activators such as VP64, or VP16, deaminases such as for example cytosine deaminase 1 (pCDM), adenosine deaminase, such as TadA ou TadA7.10, Apolipoprotein B mRNA editing enzyme catalytic polypeptide-like (APOBEC), Activation-induced cytidine deaminase (AICDA), DddA (double strand DNA cytidine deaminase) that may be associated to Uracil Glycosylase Inhibitors (UGI), nickases derived from Cas9 or Cpf1, transposase, integrase, topoisomerase and reverse transcriptase (e.g. Moloney murine leukemia virus RT enzyme), their functional mutants, variants or derivatives thereof.

Exemplary polypeptides sequences that can be included in the TALE fusion proteins of the present invention are listed in Table 3 (SEQ ID NO: 109 to 137).

TABLE 3
exemplary catalytic domains of the TALE proteins of the present invention

| GENBANK/ | | SEQ | |
|---|---|---|---|
| SWISS-PROT | | ID | |
| ID | NAME | NO# | Polypeptide Sequence |
| P14870 | Fok-1 | 109 | QLVKSELEEKKSELRHKLKYVPHEYIELIEIARNSTQDRILEMKVM |
| | | | EFFMKVYGYRGKHLGGSRKPDGAIYTVGSPIDYGVIVDTKAYSG |
| | | | GYNLPIGQADEMQRYVEENQTRNKHINPNEWWKVYPSSVTEFK |
| | | | FLFVSGHFKGNYKAQLTRLNHITNCNGAVLSVEELLIGGEMIKAG |
| | | | TLTLEEVRRKFNNGEINF |
| Q4VWW5 | I-Onu1 | 110 | MENNIKLENSCCALNKNKSNIFNKYINNKYKLVPFKTLVNYVNEP |
| | | | RYIPSEFKEWNNSIYYFNFNNIKNLPVYDINLNKLLKSYFDLYFISKNK |
| | | | NNKFISIIKKKQRYSLNKIFISKADLKHTSSKIIITIYIFNRERIILIK |
| | | | NLIFLYSLHFKTKSYLEKNKNLFFFESLKKKLNNKYEIFNKLKLNFN |
| | | | LNNLKFKDIMLYKLSKLLSKFYNKKVEFNIINLNSYKYNSDILTDIFK |
| | | | KKVVNPNSKLIKIMKFIGKKSLRASIGKTGDNYMDKTRISKSINYD |
| | | | LIPNKYKNLNISLIIENINFNETIKNIYNISNDTNENIIYNSIKYKLVVG |
| | | | VRLAIKGRLTKRYRADRSKLYSKTVGNLQNIDSSFKGLSSKLYRN |
| | | | KLNSNMQYTLDVYKRHVGAYAVKGWISGRSYSTSAYMSRRESI |
| | | | NPWILTGFADAEGSFLLRIRNNNKSSVGYSTELGFQITLHNKDKSI |
| | | | LENIQSTWKVGVIANSGDNAVSLKVTRFEDLKVIIDHFEKYPLITQ |
| | | | KLGDYMLFKQAFCVMENKEHLKINGIKELVRIKAKLNWGLTDELK |
| | | | KAFPEIISKERSLINKNIPNFKWLAGFTSGEGCFFVNLIKSKSKLG |
| | | | VQVQLVFSITQHIKDKNLMNSLITYLGCGYIKEKNKSEFSWLDFV |
| | | | VTKFSDINDKIIPVFQENTLIGVKLEDFEDWCKVAKLIEEKKHLTES |
| | | | GLDEIKKIKLNMNKGRVF |
| P05725.1 | I-CreI | 111 | MNTKYNKEFLLYLAGFVDGDGSIIAQIKPNQSYKFKHQLSLAFQV |
| | | | TQKTQRRWFLDKLVDEIGVGYVRDRGSVSDYILSEIKPLHNFLTQ |
| | | | LQPFLKLKQKQANLVLKIIWRLPSAKESPDKFLEVCTWVDQIAAL |
| | | | NDSKTRKTTSETVRAVLDSLSEKKKSSP |
| AAK09365.1 | I-BmoI | 112 | MKSGVYKITNKNTGKFYIGSSEDCESRLKVHFRNLKNNRHINRYL |
| | | | NNSFNKHGEQVFIGEVIHILPIEEAIAKEQWYIDNFYEEMYNISKS |
| | | | AYHGGDLTSYHPDKRNIILKRADSLKKVYLKMTSEEKAKRWQCV |
| | | | QGENNPMFGRKHTETTKLKISNHNKLYYSTHKNPFKGKKHSEES |
| | | | KTKLSEYASQRVGEKNPFYGKTHSDEFKTYMSKKFKGRKPKNS |
| | | | RPVIIDGTEYESATEASRQLNVVPATILHRIKSKNEKYSGYFYK |
| P34081.1 | I-HmuI | 113 | MEWKDIKGYEGHYQVSNTGEVYSIKSGKTLKHQIPKDGYHRIGL |
| | | | FKGGKGKTFQVHRLVAIHFCEGYEEGLVVDHKDGNKDNNLSTN |
| | | | LRWVTQKINVENQMSRGTLNVSKAQQIAKIKNQKPIIVISPDGIEK |
| | | | EYPSTKCACEELGLTRGKVTDVLKGHRIHHKGYTFRYKLNG |
| P13299.2 | I-TevI | 114 | MKSGIYQIKNTLNNKVYVGSAKDFEKRWKRHFKDLEKGCHSSIK |
| | | | LQRSFNKHGNVFECSILEEIPYEKDLIIERENFWIKELNSKINGYNI |
| | | | ADATFGDTCSTHPLKEEIIKKRSETVKAKMLKLGPDGRKALYSKP |
| | | | GSKNGRWNPETHKFCKCGVRIQTSAYTCSKCRNRSGENNSFFN |
| | | | HKHSDITKSKISEKMKGKKPSNIKKISCDGVIFDCAADAARHFKIS |
| | | | SGLVTYRVKSDKWNWFYINA |
| V9H5A9 | Clo51 | 115 | EGIKSNISLLKDELRGQISHISHEYLSLIDLAFDSKQNRLFEMKVLE |
| | | | LLVNEYGFKGRHLGGSRKPDGIVYSTTLEDNFGIIVDTKAYSEGY |
| | | | SLPISQADEMERYVRENSNRDEEVNPNKWWENFSEEVKKYYFV |
| | | | FISGSFKGKFEEQLRRLSMTTGVNGSAVNVVNLLLGA |
| | | | EKIRSGEMTIEELERAMFNNSEFILKY |
| CAA45962.1 | NucA | 116 | MGICGKLGVAALVALIVGCSPVQSQVPPLTELSPSISVHLLLGNP |
| | | | SGATPTKLTPDNYLMVKNQYALSYNNSKGTANWVAWQLNSSW |
| | | | LGNAERQDNFRPDKTLPAGWVRVTPSMYSGSGYDRGHIAPSAD |
| | | | RTKTTEDNAATFLMTNMMPQTPDNNRNTWGNLEDYCRELVSQ |
| | | | GKELYIVAGPNGSLGKPLKGKVTVPKSTWKIVVVLDSPGSGLEGI |
| | | | TANTRVIAVNIPNDPELNNDWRAYKVSVDELESLTGYDFLSNVSP |
| | | | NIQTSIESKVDN |
| Q53H47.1 | Metnase | 117 | MAEFKEKPEAPTEQLDVACGQENLPVGAWPPGAAPAPFQYTPD |
| | | | HVVGPGADIDPTQITFPGCICVKTPCLPGTCSCLRHGENYDDNS |
| | | | CLRDIGSGGKYAEPVFECNVLCRCSDHCRNRVVQKGLQFHFQV |
| | | | FKTHKKGWGLRTLEFIPKGRFVCEYAGEVLGFSEVQRRIHLQTK |
| | | | SDSNYIIAIREHVYNGQVMETFVDPTYIGNIGRFLNHSCEPNLLMI |
| | | | PVRIDSMVPKLALFAAKDIVPEEELSYDYSGRYLNLTVSEDKERL |
| | | | DHGKLRKPCYCGAKSCTAFLPFDSSLYCPVEKSNISCGNEKEPS |
| | | | MCGSAPSVFPSCKRLTLETMKMMLDKKQIRAIFLFEFKMGRKAA |
| | | | ETTRNINNAFGPGTANERTVQWWFKKFCKGDESLEDEERSGRP |
| | | | SEVDNDQLRAIIEADPLTTTREVAEELNVNHSTVVRHLKQIGKVK |
| | | | KLDKWVPHELTENQKNRRFEVSSSLILRNHNEPFLDRIVTCDEK |
| | | | WILYDNRRRSAQWLDQEEAPKHFPKPILHPKKVMVTIWWSAAG |
| | | | LIHYSFLNPGETITSEKYAQEIDEMNQKLQRLQLALVNRKGPILLH |
| | | | DNARPHVAQPTLQKLNELGYEVLPHPPYSPDLLPTNYHVFKHLN |
| | | | NFLQGKRFHNQQDAENAFQEFVESQSTDFYATGINQLISRWQK |
| | | | CVDCNGSYFD |
| Q9BQ50.1 | Human TREX2 | 118 | MGRAGSPLPRSSWPRMDDCGSRSRCSPTLCSSLRTCYPRGNIT |
| | | | MSEAPRAETFVFLDLEATGLPSVEPEIAELSLFAVHRSSLENPEH |
| | | | DESGALVLPRVLDKLTLCMCPERPFTAKASEITGLSSEGLARCRK |
| | | | AGFDGAVVRTLQAFLSRQAGPICLVAHNGFDYDFPLLCAELRRL |
| | | | GARLPRDTVCLDTLPALRGLDRAHSHGTRARGRQGYSLGSLFH |
| | | | RYFRAEPSAAHSAEGDVHTLLLIFLHRAAELLAWADEQARGWAH |
| | | | IEPMYLPPDDPSLEA |
| AAH63664.1 | Human DNA2 | 119 | FAIPASRMEQLNELELLMEKSFWEEAELPAELFQKKVVASFPRT |
| | | | VLSTGMDNRYLVLAVNTVQNKEGNCEKRLVITASQSLENKELCIL |
| | | | RNDWCSVPVEPGDIIHLEGDCTSDTWIIDKDFGYLILYPDMLISGT |
| | | | SIASSIRCMRRAVLSETFRSSDPATRQMLIGTVLHEVFQKAINNS |
| | | | FAPEKLQELAFQTIQEIRHLKEMYRLNLSQDEIKQEVEDYLPSFC |
| | | | KWAGDFMHKNTSTDFPQMQLSLPSDNSKDNSTCNIEVVKPMDI |
| | | | EESIWSPRFGLKGKIDVTVGVKIHRGYKTKYKIMPLELKTGKESN |
| | | | SIEHRSQVVLYTLLSQERRADPEAGLLLYLKTGQMYPVPANHLD |
| | | | KRELLKLRNQMAFSLFHRISKSATRQKTQLASLPQIIEEEKTCKYC |
| | | | SQIGNCALYSRAVEQQMDCSSVPIVMLPKIEEETQHLKQTHLEYF |
| | | | SLWCLMLTLESQSKDNKKNHQNIWLMPASEMEKSGSCIGNLIRM |
| | | | EHVKIVCDGQYLHNFQCKHGAIPVTNLMAGDRVIVSGEERSLFA |
| | | | LSRGYVKEINMTTVTCLLDRNLSVLPESTLFRLDQEEKNCDIDTP |
| | | | LGNLSKLMENTFVSKKLRDLIIDFREPQFISYLSSVLPHDAKDTVA |
| | | | CILKGLNKPQRQAMKKVLLSKDYTLIVGMPGTGKTTTICTLVPAP |
| | | | EQVEKGGVSNVTEAKLIVFLTSIFVKAGCSPSDIGIIAPYRQQLKII |
| | | | NDLLARSIGMVEVNTVDKYQGRDKSIVLVSFVRSNKDGTVGELL |
| | | | KDWRRLNVAITRAKHKLILLGCVPSLNCYPPLEKLLNHLNSEKLII |
| | | | DLPSREHESLCHILGDFQRE |
| P68336 | VP16 (Human | 120 | MDLLVDDLFADRDGVSPPPPRPAGGPKNTPAAPPLYATGRLSQ |
| | herpesvirus 2) | | AQLMPSPPMPVPPAALFNRLLDDLGFSAGPALCTMLDTWNEDL |
| | | | FSGFPTNADMYRECKFLSTLPSDVIDWGDAHVPERSPIDIRAHG |
| | | | DVAFPTLPATRDELPSYYEAMAQFFRGELRAREESYRTVLANFC |
| | | | SALYRYLRASVRQLHRQAHMRGRNRDLREMLRTTIADRYYRET |
| | | | ARLARVLFLHLYLFLSREILWAAYAEQMMRPDLFDGLCCDLESW |
| | | | RQLACLFQPLMFINGSLTVRGVPVEARRLRELNHIREHLNLPLVR |
| | | | SAAAEEPGAPLTTPPVLQGNQARSSGYFMLLIRAKLDSYSSVAT |
| | | | SEGESVMREHAYSRGRTRNNYGSTIEGLLDLPDDDDAPAEAGL |
| | | | VAPRMSFLSAGQRPRRLSTTAPITDVSLGDELRLDGEEVDMTPA |
| | | | DALDDFDLEMLGDVESPSPGMTHDPVSYGALDVDDFEFEQMFT |
| | | | DAMGIDDFGG |
| P68398 | Cytidine | 121 | MSEVEFSHEYWMRHALTLAKRAWDEREVPVGAVLVHNNRVIGE |
| | deaminase: tRNA- | | GWNRPIGRHDPTAHAEIMALRQGGLVMQNYRLIDATLYVTLEPC |
| | specific adenosine | | VMCAGAMIHSRIGRVVFGARDAKTGAAGSLMDVLHHPGMNHRV |
| | deaminase (TadA) | | EITEGILADECAALLSDFFRMRRQEIKAQKKAQSSTD |
| Q923K9 | Cytidine | 122 | MSSETGPVAVDPTLRRRIEPHEFEVFFDPRELRKETCLLYEINW |
| | deaminase: C->U- | | GGRHSIWRHTSQNTNKHVEVNFIEKFTTERYFCPNTRCSITWFL |
| | editing enzyme | | SWSPCGECSRAITEFLSRYPHVTLFIYIARLYHHADPRNRQGLRD |
| | APOBEC-1- | | LISSGVTIQIMTEQESGYCWRNFVNYSPSNEAHWPRYPHLWVRL |
| | Rattus norvegicus | | YVLELYCIILGLPPCLNILRRKQPQLTFFTIALQSCHYQRLPPHILW |
| | | | ATGLK |
| | Cytidine | 123 | MDPQRLRQWPGPGPASRGGYGQRPRIRNPEEWFHELSPRTFS |
| | deaminase: DNA | | FHFRNLRFASGRNRSYICCQVEGKNCFFQGIFQNQVPPDPPCH |
| | dC->dU-editing | | AELCFLSWFQSWGLSPDEHYYVTWFISWSPCCECAAKVAQFLE |
| | enzyme APOBEC- | | ENRNVSLSLSAARLYYFWKSESREGLRRLSDLGAQVGIMSFQDF |
| | 3B-Sus scrofa | | QHCWNNFVHNLGMPFQPWKKLHKNYQRLVTELKQILREEPATY |
| | | | GSPQAQGKVRIGSTAAGLRHSHSHTRSEAHLRPNHSSRQHRIL |
| | | | NPPREARARTCVLVDASWICYR |
| | Cytidine | 124 | MADSSEKMRGQYISRDTFEKNYKPIDGTKEAHLLCEIKWGKYGK |
| | deaminase: C->U- | | PWLHWCQNQRMNIHAEDYFMNNIFKAKKHPVHCYVTWYLSWS |
| | editing enzyme | | PCADCASKIVKFLEERPYLKLTIYVAQLYYHTEEENRKGLRLLRS |
| | APOBEC-1- | | KKVIIRVMDISDYNYCWKVFVSNQNGNEDYWPLQFDPWVKENY |
| | Alligator | | SRLLDIFWESKCRSPNPW |
| | mississippiensis | | |
| | Cytidine | 125 | MTSEKGPSTGDPTLRRRIESWEFDVFYDPRELRKETCLLYEIKW |
| | deaminase: C->U- | | GMSRKIWRSSGKNTTNHVEVNFIKKFTSERRFHSSISCSITWFLS |
| | editing enzyme | | WSPCWECSQAIREFLSQHPGVTLVIYVARLFWHMDQRNRQGLR |
| | APOBEC-1- | | DLVNSGVTIQIMRASEYYHCWRNFVNYPPGDEAHWPQYPPLW |
| | Pongo pygmaeus | | MMLYALELHCIILSLPPCLKISRRWQNHLAFFRLHLQNCHYQTIPP |
| | | | HILLATGLIHPSVTWR |
| Q9GZX7 | Cytidine | 126 | MDSLLMNRRKFLYQFKNVRWAKGRRETYLCYVVKRRDSATSFS |
| | deaminase: | | LDFGYLRNKNGCHVELLFLRYISDWDLDPGRCYRVTWFTSWSP |
| | Single-stranded | | CYDCARHVADFLRGNPNLSLRIFTARLYFCEDRKAEPEGLRRLH |
| | DNA cytosine | | RAGVQIAIMTFKDYFYCWNTFVENHERTFKAWEGLHENSVRLSR |
| | deaminase | | QLRRILLPLYEVDDLRDAFRTLGL |
| | (AICDA, AID)- | | |
| Q9NRW3 | Cytidine | 127 | MNPQIRNPMKAMYPGTFYFQFKNLWEANDRNETWLCFTVEGIK |
| | deaminase: DNA | | RRSVVSWKTGVFRNQVDSETHCHAERCFLSWFCDDILSPNTKY |
| | dC->dU-editing | | QVTWYTSWSPCPDCAGEVAEFLARHSNVNLTIFTARLYYFQYPC |
| | enzyme APOBEC- | | YQEGLRSLSQEGVAVEIMDYEDFKYCWENFVYNDNEPFKPWKG |
| | 3C | | LKTNFRLLKRRLRESLQ |
| Q96AK3 | “Cytidine | 128 | MNPQIRNPMERMYRDTFYDNFENEPILYGRSYTWLCYEVKIKRG |
| | deaminase: DNA | | RSNLLWDTGVFRGPVLPKRQSNHRQEVYFRFENHAEMCFLSW |
| | dC->dU-editing | | FCGNRLPANRRFQITWFVSWNPCLPCVVKVTKFLAEHPNVTLTI |
| | enzyme APOBEC- | | SAARLYYYRDRDWRWVLLRLHKAGARVKIMDYEDFAYCWENFV |
| | 3D isoform 1 | | CNEGQPFMPWYKFDDNYASLHRTLKEILRNPMEAMYPHIFYFHF |
| | | | KNLLKACGRNESWLCFTMEVTKHHSAVFRKRGVFRNQVDPETH |
| | | | CHAERCFLSWFCDDILSPNTNYEVTWYTSWSPCPECAGEVAEF |
| | | | LARHSNVNLTIFTARLCYFWDTDYQEGLCSLSQEGASVKIMGYK |
| | | | DFVSCWKNFVYSDDEPFKPWKGLQTNFRLLKRRLREILQ |
| Q8IUX4 | DNA dC->dU- | 129 | MKPHFRNTVERMYRDTFSYNFYNRPILSRRNTVWLCYEVKTKG |
| | editing enzyme | | PSRPRLDAKIFRGQVYSQPEHHAEMCFLSWFCGNQLPAYKCFQ |
| | APOBEC-3F | | ITWFVSWTPCPDCVAKLAEFLAEHPNVTLTISAARLYYYWERDY |
| | isoform a | | RRALCRLSQAGARVKIMDDEEFAYCWENFVYSEGQPFMPWYK |
| | | | FDDNYAFLHRTLKEILRNPMEAMYPHIFYFHFKNLRKAYGRNES |
| | | | WLCFTMEVVKHHSPVSWKRGVFRNQVDPETHCHAERCFLSWF |
| | | | CDDILSPNTNYEVTWYTSWSPCPECAGEVAEFLARHSNVNLTIF |
| | | | TARLYYFWDTDYQEGLRSLSQEGASVEIMGYKDFKYCWENFVY |
| | | | NDDEPFKPWKGLKYNFLFLDSKLQEILE |
| Q9HC16 | Cytidine | 130 | MKPHFRNTVERMYRDTFSYNFYNRPILSRRNTVWLCYEVKTKG |
| | deaminase: DNA | | PSRPPLDAKIFRGQVYSELKYHPEMRFFHWFSKWRKLHRDQEY |
| | dC->dU-editing | | EVTWYISWSPCTKCTRDMATFLAEDPKVTLTIFVARLYYFWDPD |
| | enzyme APOBEC- | | YQEALRSLCQKRDGPRATMKIMNYDEFQHCWSKFVYSQRELFE |
| | 3G isoform 1 | | PWNNLPKYYILLHIMLGEILRHSMDPPTFTFNFNNEPWVRGRHE |
| | | | TYLCYEVERMHNDTWVLLNQRRGFLCNQAPHKHGFLEGRHAEL |
| | | | CFLDVIPFWKLDLDQDYRVTCFTSWSPCFSCAQEMAKFISKNKH |
| | | | VSLCIFTARIYDDQGRCQEGLRTLAEAGAKISIMTYSEFKHCWDT |
| | | | FVDHQGCPFQPWDGLDEHSQDLSGRLRAILQNQEN |
| O65896 | Cytidine | 131 | MDKPSFVIQSKEAESAAKQLGVSVIQLLPSLVKPAQSYARTPISK |
| | deaminase: | | FNVAVVGLGSSGRIFLGVNVEFPNLPLHHSIHAEQFLVTNLTLNG |
| | cytidine | | ERHLNFFAVSAAPCGHCRQFLQEIRDAPEIKILITDPNNSADSDS |
| | deaminase 1 | | AADSDGFLRLGSFLPHRFGPDDLLGKDHPLLLESHDNHLKISDLD |
| | (Arabidopsis | | SICNGNTDSSADLKQTALAAANRSYAPYSLCPSGVSLVDCDGKV |
| | thaliana) | | YRGWYMESAAYNPSMGPVQAALVDYVANGGGGGYERIVGAVL |
| | | | VEKEDAVVRQEHTARLLLETISPKCEFKVFHCYEA |
| P03355 | reverse | 132 | TLNIEDEHRLHETSKEPDVSLGSTWLSDFPQAWAETGGMGLAV |
| | transcriptase p80 | | RQAPLIIPLKATSTPVSIKQYPMSQEARLGIKPHIQRLLDQGILVPC |
| | RT [Moloney | | QSPWNTPLLPVKKPGTNDYRPVQDLREVNKRVEDIHPTVPNPY |
| | murine leukemia | | NLLSGLPPSHQWYTVLDLKDAFFCLRLHPTSQPLFAFEWRDPE |
| | virus] | | MGISGQLTWTRLPQGFKNSPTLFDEALHRDLADFRIQHPDLILLQ |
| | | | YVDDLLLAATSELDCQQGTRALLQTLGNLGYRASAKKAQICQKQ |
| | | | VKYLGYLLKEGQRWLTEARKETVMGQPTPKTPRQLREFLGTAG |
| | | | FCRLWIPGFAEMAAPLYPLTKTGTLFNWGPDQQKAYQEIKQALL |
| | | | TAPALGLPDLTKPFELFVDEKQGYAKGVLTQKLGPWRRPVAYLS |
| | | | KKLDPVAAGWPPCLRMVAAIAVLTKDAGKLTMGQPLVILAPHAV |
| | | | EALVKQPPDRWLSNARMTHYQALLLDTDRVQFGPVVALNPATLL |
| | | | PLPEEGLQHNCLDILAEAHGTRPDLTDQPLPDADHTWYTDGSSL |
| | | | LQEGQRKAGAAVTTETEVIWAKALPAGTSAQRAELIALTQALKM |
| | | | AEGKKLNVYTDSRYAFATAHIHGEIYRRRGLLTSEGKEIKNKDEIL |
| | | | ALLKALFLPKRLSIIHCPGHQKGHSAEARGNRMADQAARKAAITE |
| | | | TPDTSTLLIENSSPSGGSKRTADGSEFE |
| Q9B086 | Bxb1 integrase | 133 | MRALVVIRLSRVTDATTSPERQLESCQQLCAQRGWDVVGVAED |
| | | | LDVSGAVDPFDRKRRPNLARWLAFEEQPFDVIVAYRVDRLTRSI |
| | | | RHLQQLVHWAEDHKKLVVSATEAHFDTTTPFAAVVIALMGTVAQ |
| | | | MELEAIKERNRSAAHFNIRAGKYRGSLPPWGYLPTRVDGEWRL |
| | | | VPDPVQRERILEVYHRVVDNHEPLHLVAHDLNRRGVLSPKDYFA |
| | | | QLQGREPQGREWSATALKRSMISEAMLGYATLNGKTVRDDDGA |
| | | | PLVRAEPILTREQLEALRAELVKTSRAKPAVSTPSLLLRVLFCAVC |
| | | | GEPAYKFAGGGRKHPRYRCRSMGFPKHCGNGTVAMAEWDAF |
| | | | CEEQVLDLLGDAERLEKVWVAGSDSAVELAEVNAELVDLTSLIG |
| | | | SPAYRAGSPQREALDARIAALAARQEELEGLEARPSGWEWRET |
| | | | GQRFGDWWREQDTAAKNTWLRSMNVRLTFDVRGGLTRTIDFG |
| | | | DLQEYEQHLRLGSVVERLHTGMS |
| PODUH5 | DddA N-half | 134 | GSYALGPYQISAPQLPAYNGQTVGTFYYVNDAGGLESKVFSSG |
| | | | GPTPYPNYANAGHVEGQSALFMRDNGISEGLVFHNNPEGTCGF |
| | | | CVNMTETLLPENAKMTVVPPEG |
| PODUH5 | DddA C-half | 135 | AIPVKRGATGETKVFTGNSNSPKSPTKGGC |
| P14739 | canonical UGI | 136 | NLSDIIEKETGKQLVIQESILMLPEEVEEVIGNKPESDILVHTAYDE |
| | (uracil-DNA- | | STDENVMLLTSDAPEYKPWALVIQDSNGENKIKML |
| | glycosylase | | |
| | inhibitor) | | |
| P11387 | (subdomain of) | 137 | DGKLKKPKNKDKDKKVPEPDNKKKKPKKEEEQKWKWWEEERY |
| | DNA | | PEGIKWKFLEHKGPVFAPPYEPLPENVKFYYDGKVMKLSPKAEE |
| | topoisomerase 1 | | VATFFAKMLDHEYTTKEIFRKNFFKDWRKEMTNEEKNIITNLSKC |
| | | | DFTQMSQYFKAQTEARKQMSKEEKLKIKEENEKLLKEYGFCIMD |
| | | | NHKERIANFKIEPPGLFRGRGNHPKMGMLKRRIMPEDIIINCSKD |
| | | | AKVPSPPPGHKWKEVRHDNKVTWLVSWTENIQGSIKYIMLNPSS |
| | | | RIKGEKDWQKYETARRLKKCVDKIRNQYREDWKSKEMKVRQRA |
| | | | VALYFIDKLALRAGNEKEEGETADTVGCCSLRVEHINLHPELDGQ |
| | | | EYWVEFDFLGKDSIRYYNKVPVEKRVFKNLQLFMENKQPEDDLF |
| | | | DRLNTGILNKHLQDLMEGLTAKVFRTYNASITLQQQLKELTAPDE |
| | | | NIPAKILSYNRANRAVAILCNHQRAPPKTFEKSMMNLQTKIDAKK |
| | | | EQLADARRDLKSAKADAKVMKDAKTKKVVESKKKAVQRLEEQL |
| | | | MKLEVQATDREENKQIALGTSKLNYLDPRITVAWCKKWGVPIEKI |
| | | | YNKTQREKFAWAIDMADEDYEF |

In another embodiment, the TALE fusion protein according to the present invention comprises a catalytic domain that is a polypeptide comprising an amino acid sequence having at least 80%, preferably at least 90%, more preferably at least 95% identity with any of SEQ ID NO: 109 to 137.

Since gene editing reagents can cause unintended interruptions in the genome, gene editing is crucial and as multiplex methods become more widely used, the likelihood of off-targets and the downstream consequences of such off-target activity grow. Minimizing such undesired cleavage (off-targets) is a matter of utmost importance for any genome-engineering applications, especially in the therapeutic domain. Undesired double-stranded breaks in the genome may lead to chromosome translocation, and cellular toxicity [Cantoni O., et al. (1996) Cytotoxic impact of DNA single vs double strand breaks in oxidatively injured cells. Arch Toxicol Suppl 18:223-235]. There are currently a variety of techniques available to predict and quantify off-target by analysing secondary target locations and establish on-target/off target ratios, such as those described by Tsai S., et al. [CIRCLE-seq: a highly sensitive in vitro screen for genome-wide CRISPR-Cas9 nuclease off-targets (2017) Nat Methods 14 (6): 607-614], Hockemeyer D, et al. [Genetic engineering of human pluripotent cells using TALE nucleases (2011) Nat. Biotechnol. 29 (8): 731- and Wienert B, et al. [Unbiased detection of CRISPR off-targets in vivo using DISCOVER-Seq (2019) Science 364 (6437): 286-289].

As previously mentioned, TALE proteins have a well-defined DNA base-pair choice, offering a basic strategy for scientific researchers and engineers to design and construct TALE fusion proteins for genome alteration. A TALE repeat tandem is responsible for recognizing individual DNA base pairs. Such tandem is made up of a pair of alpha helices linked by a loop of three-residue of RVDs in the shape of a solenoid. For the creation of TALE proteins with variable precision and binding affinity, the six conventional RVDs (NG, HD, NI, NK, NH, and NN) are frequently used. HD and NG are associated with cytosine (C) and thymine (T) respectively. These associations are strong and exclusive [Streubel J, et al. (2012) TAL effector RVD specificities and efficiencies. Nat Biotechnol 30 (7): 593-595]. NN is a degenerate RVD usually showing binding affinity for both guanine (G) and adenine (A), but its specificity for guanine is reported to be stronger. RVD NI binds with A and NK binds with G. These associations are exclusive but the binding affinity between these pairs is less due to which they are considered weak. Therefore, it is recommended to use RVD NH which binds with G with medium affinity. It is also worth noting that the binding affinity of TALE is influenced by the methylation status of the target DNA sequence.

The TALEN code is degenerate, which means that certain RVDs can bind to multiple nucleotides with a diverse spectrum of efficiency. The binding ability of the NN (for A and G) and NS (A, C, and G) repeat variable di-residue empowers the TALE proteins to encode degeneracy for the target DNA. This degeneracy may although be useful in targeting hyper variable sites. TALE proteins technology is the only known genome editing tool which can be engineered in a way that can be easily used for the escape mutations in a genome. This unique feature make them a more flexible and reliable tool in the field of genome editing specifically in clinical applications to tolerate predicted mutations [Strong CL, et al. (2015) Damaging the integrated HIV proviral DNA with TALENs. PLOS One 10 (5): e0125652.]

A typical TALE protein usually consists of 18 repeats of 34 amino acids. A TALEN pair must bind to the target site on opposite sides, separated by a “spacer” of 14-20 nucleotides as an offset since Fokl requires dimerization for operation. As a whole, such a long (approximately 36 bp) DNA binding site is predicted to appear in genomes as being very rare.

### Development of Specific TALE-Nucleases

By following the above teachings, highly specific TALE-nucleases can be produced according to the present invention allowing high degree of cleavage specificity and low cytotoxicity in diverse cell types, especially plant or mammalian cells.

According to some embodiments, the TALE-fusion protein of the present invention is a TALE-nuclease obtained by fusion of a TALE protein as described herein with the nuclease catalytic domain of a non-specific nuclease, such as Fok-1 (SEQ ID NO: 109) or Tev-1 (SEQ ID NO: 114) as described with classical TALE scaffolds for instance in Beurdeley, M. et al. [Compact designer TALENs for efficient genome engineering (2013) Nat Commun 4:1762]. In preferred embodiments, said nuclease catalytic domain is Fok1, i.e. comprises a polypeptide showing at least 80% identity with SEQ ID NO.1, and more preferably comprising at least one of the amino acid substitutions: 13, 52, 57, 59, 61, 65, 84, 85, 88, 91, 92, 95, 98, 103, 109, 110, 111, 113, 119, 143, 148, 152, 158, 159, 160, 167, 169, 170 and 194 into SEQ ID NO: 109, as illustrated herein in the Examples. Preferred substitutions are introduced at positions 84, 85, 88, 95, 98, 91, 103, 109, 148, 152 and 158, and most preferred ones are in positions 84, 88, 91, 103 and 152.

According to some embodiments, the TALE-fusion protein of the present invention is a TALE-nuclease obtained by fusion of a TALE protein as described herein with a nickase, in particular a Cas9 nickase. Such Cas9 nickase are generally Cas9 proteins which are mutated in their RuvC or HNH domains, for instance by introducing mutations D10A in RuvC and H840A in HNH. In general, TALE-Cas9 nickase fusions are used by pairs as formerly described with classical TALE scaffolds by Guilinger, J., et al. [Fusion of catalytically inactive Cas9 to Fokl nuclease improves the specificity of genome modification (2014) Nat. Biotechnol. 32, 577-582].

In some other embodiments, the TALE-fusion protein of the present invention is a TALE-nuclease obtained by fusion of a TALE protein as described herein with a specific nuclease, preferably a customized rare-cutting endonuclease, such as a meganuclease variant. In preferred embodiments, said rare-cutting endonuclease can be a variant of LADLIDADG, such as I-crel or I-Onul, as previously described for instance in EP3320910 and EP3004338.

On another hand, a TALE-nuclease according to the present invention has also the ability to efficiently manipulate mtDNA (mitochondrial DNA) as a treatment for treating human mitochondrial diseases triggered by mitochondrial pathogenic mutations. So called “Mito-TALEN” (mitochondrial-targeted TALENs) have been proven to be effectively treating human mitochondrial disorders affected by mtDNA mutations, such as Leber's hereditary optic neuropathy, ataxia, neurogenic muscle fatigue, and retinal pigmentosa [Gammage, P.A., et al. (2018) Mitochondrial Genome Engineering: The Revolution May Not Be CRISPR-Ized. Trends in Genetics, 34 (2): 101-110]. Plastid engineering has also demonstrated competent results in varieties of plants for crop improvements [Piatek AA, Lenaghan SC, Neal Stewart C. (2018) Advanced editing of the nuclear and plastid genomes in plants. Plant Sci 273:42-49].

Many examples of TALE-nuclease as per the present invention are herein described to be used as therapeutic reagent to induce highly specific cleavage in a selection of genes in human cells, especially blood cells. More particularly, improved TALE nuclease reagents have been synthetized and tested pursuant to the present teachings in order to cleave gene targets in primary cells, especially in T-cells or NK cells, such as TCRalpha, B2m, PD1, CTLA4, CISH, LAG3, TGFBRII, TIGIT, CD38, IgH, GADPH and CCR5.

The polypeptide sequences of these TALE proteins obtained as per the present invention, as well as their target sequences (polynucleotide sequence spanning the two left and right heterodimeric binding sites) are listed in Table 4 and 5 below, as well as in Tables 5 and 6 in the example section.

TABLE 4
Examples of TALE proteins useful in therapy

| TALE- | | SEQ |
|---|---|---|
| nuclease | | ID |
| designation | TALE Polypeptide sequence | NO: # |
| TALE V2 | MGDPKKKRKVIDIADLRTLGYSQQQQEKIKPKVRSTVAQHHEALVGHGFTHAHI | 138 |
| CTLA4 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASHDGGKQALETVQRLLPVLC | |
| | QDHGLTPDQVVAIASNIGGKQALETVQALLPVLCQDHGLTPDQVVAIASHDGG | |
| | KQALETVQRLLPVLCQDHGLTPDQVVAIASNGGGKQALETVQRLLPVLCQDHG | |
| | LTPDQVVAIASNGGGKQALETVQRLLPVLCQDHGLTPDQVVAIASNNGGKQAL | |
| | ETVQRLLPVLCQDHGLTPDQVVAIASNIGGKQALETVQALLPVLCQDHGLTPD | |
| | QVVAIASNGGGKQALETVQRLLPVLCQDHGLTPDQVVAIASNGGGKQALETVQ | |
| | RLLPVLCQDHGLTPDQVVAIASNGGGKQALETVQRLLPVLCQDHGLTPDQVVA | |
| | IASHDGGKQALETVQRLLPVLCQDHGLTPDQVVAIASHDGGKQALETVQRLLP | |
| | VLCQDHGLTPDQVVAIASNIGGKQALETVQALLPVLCQDHGLTPDQWVAIASHD | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 139 |
| CTLA4 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNGGGKQALETVQRLLPVLCQDHGLTPDQVVAIASNIGG | |
| | KQALETVQALLPVLCQDHGLTPDQVVAIASNNGGKQALETVQRLLPVLCQDHG | |
| | LTPDQVVAIASNIGGKQALETVQALLPVLCQDHGLTPDQVVAIASNGGGKQALE | |
| | TVQRLLPVLCQDHGLTPDQVVAIASNNGGKQALETVQRLLPVLCQDHGLTPDQ | |
| | VVAIASNIGGKQALETVQALLPVLCQDHGLTPDQVVAIASNGGGKQALETVQRL | |
| | LPVLCQDHGLTPDQVVAIASNGGGKQALETVQRLLPVLCQDHGLTPDQVVAIA | |
| | SHDGGKQALETVQRLLPVLCQDHGLTPDQVVAIASHDGGKQALETVQRLLPVL | |
| | CQDHGLTPDQVVAIASNIGGKQALETVQALLPVLCQDHGLTPDQVVAIASNGG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 140 |
| CISH R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NIGGKQALETVQALLPVLCQDHGLTPDQVVAIASHDGGKQALETVQRLLPVLC | |
| | QDHGLTPDQVVAIASNIGGKQALETVQALLPVLCQDHGLTPDQVVAIASNNGG | |
| | KQALETVQRLLPVLCQDHGLTPDQVVAIASHDGGKQALETVQRLLPVLCQDHG | |
| | LTPDQVVAIASNIGGKQALETVQALLPVLCQDHGLTPDQVVAIASNNGGKQALE | |
| | TVQRLLPVLCQDHGLTPDQVVAIASNGGGKQALETVQRLLPVLCQDHGLTPDQ | |
| | VVAIASNNGGKQALETVQRLLPVLCQDHGLTPDQVVAIASNNGGKQALETVQR | |
| | LLPVLCQDHGLTPDQVVAIASHDGGKQALETVQRLLPVLCQDHGLTPDQVVAI | |
| | ASNGGGKQALETVQRLLPVLCQDHGLTPDQVVAIASNNGGKQALETVQRLLPV | |
| | LCQDHGLTPDQVVAIASNNGGKQALETVQRLLPVLCQDHGLTPDQVVAIASNG | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 141 |
| CISH L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NNGGKQALETVQRLLPVLCQDHGLTPDQVVAIASHDGGKQALETVQRLLPVLC | |
| | QDHGLTPDQVVAIASNNGGKQALETVQRLLPVLCQDHGLTPDQVVAIASHDGG | |
| | KQALETVQRLLPVLCQDHGLTPDQVVAIASHDGGKQALETVQRLLPVLCQDHG | |
| | LTPDQVVAIASNGGGKQALETVQRLLPVLCQDHGLTPDQVVAIASNIGGKQALE | |
| | TVQALLPVLCQDHGLTPDQVVAIASNNGGKQALETVQRLLPVLCQDHGLTPDQ | |
| | VVAIASNGGGKQALETVQRLLPVLCQDHGLTPDQVVAIASNNGGKQALETVQR | |
| | LLPVLCQDHGLTPDQVVAIASNIGGKQALETVQALLPVLCQDHGLTPDQVVAIA | |
| | SHDGGKQALETVQRLLPVLCQDHGLTPDQVVAIASHDGGKQALETVQRLLPVL | |
| | CQDHGLTPDQVVAIASHDGGKQALETVQRLLPVLCQDHGLTPDQVVAIASNIG | |
| | GKQALETVQALLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 142 |
| LAG3 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNIGGKQALETVQALLPVLC | |
| | QDHGLTPDQVVAIASNGGGKQALETVQRLLPVLCQDHGLTPDQVVAIASNNG | |
| | GKQALETVQRLLPVLCQDHGLTPDQVVAIASHDGGKQALETVQRLLPVLCQDH | |
| | GLTPDQVVAIASNGGGKQALETVQRLLPVLCQDHGLTPDQVVAIASNNGGKQA | |
| | LETVQRLLPVLCQDHGLTPDQVVAIASHDGGKQALETVQRLLPVLCQDHGLTP | |
| | DQVVAIASHDGGKQALETVQRLLPVLCQDHGLTPDQVVAIASNIGGKQALETV | |
| | QALLPVLCQDHGLTPDQVVAIASNIGGKQALETVQALLPVLCQDHGLTPDQVV | |
| | AIASNNGGKQALETVQRLLPVLCQDHGLTPDQVVAIASNGGGKQALETVQRLL | |
| | PVLCQDHGLTPDQVVAIASNNGGKQALETVQRLLPVLCQDHGLTPDQVVAIAS | |
| | NIGGKQALETVQALLPVLCQDHGLTPQQVVAIASNGGGRPALESIVAQLSRPDP | |
| | ALAALTNDHLVALACLGGRPALDAVRRGLGDPISRSQLVKSELEEKKSELRHKL | |
| | KYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 143 |
| LAG3 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNIGGKQALETVQALLPVLCQDHGLTPDQVVAIASNNGG | |
| | KQALETVQRLLPVLCQDHGLTPDQVVAIASNNGGKQALETVQRLLPVLCQDHG | |
| | LTPDQVVAIASNIGGKQALETVQALLPVLCQDHGLTPDQVVAIASNGGGKQALE | |
| | TVQRLLPVLCQDHGLTPDQVVAIASHDGGKQALETVQRLLPVLCQDHGLTPDQ | |
| | VVAIASNGGGKQALETVQRLLPVLCQDHGLTPDQVVAIASHDGGKQALETVQR | |
| | LLPVLCQDHGLTPDQVVAIASNIGGKQALETVQALLPVLCQDHGLTPDQVVAIA | |
| | SNNGGKQALETVQRLLPVLCQDHGLTPDQVVAIASHDGGKQALETVQRLLPVL | |
| | CQDHGLTPDQVVAIASHDGGKQALETVQRLLPVLCQDHGLTPDQVVAIASNGG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | VALSQHPAALGTVAVKYQDMIAALPEATHEAIVGVGKQWSGARALEALLTVAG | 144 |
| TGFBRII R | MGDPKKKRKVIDIADLRTLGYSQQQQEKIKPKVRSTVAQHHEALVGHGFTHAHI | |
| | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNIGGKQALETVQALLPVLC | |
| | QDHGLTPDQVVAIASNNGGKQALETVQRLLPVLCQDHGLTPDQVVAIASNIGG | |
| | KQALETVQALLPVLCQDHGLTPDQVVAIASNIGGKQALETVQALLPVLCQDHGL | |
| | TPDQVVAIASNNGGKQALETVQRLLPVLCQDHGLTPDQVVAIASNIGGKQALET | |
| | VQALLPVLCQDHGLTPDQVVAIASNGGGKQALETVQRLLPVLCQDHGLTPDQV | |
| | VAIASNNGGKQALETVQRLLPVLCQDHGLTPDQVVAIASNGGGKQALETVQRL | |
| | LPVLCQDHGLTPDQVVAIASHDGGKQALETVQRLLPVLCQDHGLTPDQVVAIA | |
| | SHDGGKQALETVQRLLPVLCQDHGLTPDQVVAIASNGGGKQALETVQRLLPVL | |
| | CQDHGLTPDQVVAIASNGGGKQALETVQRLLPVLCQDHGLTPDQWVAIASHDG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 145 |
| TGFBRII L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASHDGGKQALETVQRLLPVLCQDHGLTPDQVVAIASNGG | |
| | GKQALETVQRLLPVLCQDHGLTPDQVVAIASNIGGKQALETVQALLPVLCQDH | |
| | GLTPDQVVAIASNGGGKQALETVQRLLPVLCQDHGLTPDQVVAIASNNGGKQA | |
| | LETVQRLLPVLCQDHGLTPDQVVAIASNIGGKQALETVQALLPVLCQDHGLTPD | |
| | QVVAIASNNGGKQALETVQRLLPVLCQDHGLTPDQVVAIASNNGGKQALETVQ | |
| | RLLPVLCQDHGLTPDQVVAIASNIGGKQALETVQALLPVLCQDHGLTPDQVVAI | |
| | ASNNGGKQALETVQRLLPVLCQDHGLTPDQVVAIASNGGGKQALETVQRLLPV | |
| | LCQDHGLTPDQVVAIASNIGGKQALETVQALLPVLCQDHGLTPDQVVAIASNG | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 146 |
| CCR5 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NIGGKQALETVQALLPVLCQDHGLTPDQVVAIASNGGGKQALETVQRLLPVLC | |
| | QDHGLTPDQVVAIASHDGGKQALETVQRLLPVLCQDHGLTPDQVVAIASNIGG | |
| | KQALETVQALLPVLCQDHGLTPDQVVAIASNIGGKQALETVQALLPVLCQDHGL | |
| | TPDQVVAIASNNGGKQALETVQRLLPVLCQDHGLTPDQVVAIASNGGGKQALE | |
| | TVQRLLPVLCQDHGLTPDQVVAIASNNGGKQALETVQRLLPVLCQDHGLTPDQ | |
| | VVAIASNGGGKQALETVQRLLPVLCQDHGLTPDQVVAIASHDGGKQALETVQR | |
| | LLPVLCQDHGLTPDQVVAIASNIGGKQALETVQALLPVLCQDHGLTPDQVVAIA | |
| | SNIGGKQALETVQALLPVLCQDHGLTPDQVVAIASNNGGKQALETVQRLLPVL | |
| | CQDHGLTPDQVVAIASNGGGKQALETVQRLLPVLCQDHGLTPDQVVAIASHDG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 147 |
| CCR5 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNNGGKQALETVQRLLPVLCQDHGLTPDQVVAIASNIGG | |
| | KQALETVQALLPVLCQDHGLTPDQVVAIASNGGGKQALETVQRLLPVLCQDHG | |
| | LTPDQVVAIASNNGGKQALETVQRLLPVLCQDHGLTPDQVVAIASNGGGKQAL | |
| | ETVQRLLPVLCQDHGLTPDQVVAIASNIGGKQALETVQALLPVLCQDHGLTPD | |
| | QVVAIASNGGGKQALETVQRLLPVLCQDHGLTPDQVVAIASNIGGKQALETVQ | |
| | ALLPVLCQDHGLTPDQVVAIASNIGGKQALETVQALLPVLCQDHGLTPDQVVAI | |
| | ASNGGGKQALETVQRLLPVLCQDHGLTPDQVVAIASNIGGKQALETVQALLPV | |
| | LCQDHGLTPDQVVAIASNIGGKQALETVQALLPVLCQDHGLTPDQVVAIASNG | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 148 |
| B2m L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASNIGGKQALETVQALLPVLC | |
| | QDHGLTPDQVVAIASNNGGKQALETVQRLLPVLCQDHGLTPDQVVAIASHDGG | |
| | KQALETVQRLLPVLCQDHGLTPDQVVAIASNGGGKQALETVQRLLPVLCQDHG | |
| | LTPDQVVAIASNNGGKQALETVQRLLPVLCQDHGLTPDQVVAIASNGGGKQAL | |
| | ETVQRLLPVLCQDHGLTPDQVVAIASNNGGKQALETVQRLLPVLCQDHGLTPD | |
| | QVVAIASHDGGKQALETVQRLLPVLCQDHGLTPDQVVAIASNGGGKQALETVQ | |
| | RLLPVLCQDHGLTPDQVVAIASHDGGKQALETVQRLLPVLCQDHGLTPDQVVA | |
| | IASNNGGKQALETVQRLLPVLCQDHGLTPDQVVAIASHDGGKQALETVQRLLP | |
| | VLCQDHGLTPDQVVAIASNNGGKQALETVQRLLPVLCQDHGLTPDQVVAIASH | |
| | DGGKQALETVQRLLPVLCQDHGLTPQQVVAIASNGGGRPALESIVAQLSRPDP | |
| | ALAALTNDHLVALACLGGRPALDAVRRGLGDPISRSQLVKSELEEKKSELRHKL | |
| | KYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 149 |
| B2m R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNNGGKQALETVQRLLPVLC | |
| | QDHGLTPDQVVAIASNIGGKQALETVQALLPVLCQDHGLTPDQVVAIASNGGG | |
| | KQALETVQRLLPVLCQDHGLTPDQVVAIASNIGGKQALETVQALLPVLCQDHGL | |
| | TPDQVVAIASNNGGKQALETVQRLLPVLCQDHGLTPDQVVAIASHDGGKQALE | |
| | TVQRLLPVLCQDHGLTPDQVVAIASHDGGKQALETVQRLLPVLCQDHGLTPDQ | |
| | VVAIASNGGGKQALETVQRLLPVLCQDHGLTPDQVVAIASHDGGKQALETVQR | |
| | LLPVLCQDHGLTPDQVVAIASHDGGKQALETVQRLLPVLCQDHGLTPDQVVAI | |
| | ASNIGGKQALETVQALLPVLCQDHGLTPDQVVAIASNNGGKQALETVQRLLPVL | |
| | CQDHGLTPDQVVAIASNNGGKQALETVQRLLPVLCQDHGLTPDQVVAIASHDG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 150 |
| B2m-2 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNNGGKQALETVQRLLPVLCQDHGLTPDQVVAIASNGG | |
| | GKQALETVQRLLPVLCQDHGLTPDQVVAIASNNGGKQALETVQRLLPVLCQDH | |
| | GLTPDQVVAIASNNGGKQALETVQRLLPVLCQDHGLTPDQVVAIASHDGGKQA | |
| | LETVQRLLPVLCQDHGLTPDQVVAIASHDGGKQALETVQRLLPVLCQDHGLTP | |
| | DQVVAIASNGGGKQALETVQRLLPVLCQDHGLTPDQWVAIASNGGGKQALETV | |
| | QRLLPVLCQDHGLTPDQVVAIASNIGGKQALETVQALLPVLCQDHGLTPDQVV | |
| | AIASNNGGKQALETVQRLLPVLCQDHGLTPDQVVAIASHDGGKQALETVQRLL | |
| | PVLCQDHGLTPDQVVAIASNGGGKQALETVQRLLPVLCQDHGLTPDQVVAIAS | |
| | NNGGKQALETVQRLLPVLCQDHGLTPQQVVAIASNGGGRPALESIVAQLSRPD | |
| | PALAALTNDHLVALACLGGRPALDAVRRGLGDPISRSQLVKSELEEKKSELRHK | |
| | LKYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 151 |
| B2m-2 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNIGGKQALETVQALLPVLCQDHGLTPDQVVAIASNNGG | |
| | KQALETVQRLLPVLCQDHGLTPDQVVAIASNNGGKQALETVQRLLPVLCQDHG | |
| | LTPDQVVAIASHDGGKQALETVQRLLPVLCQDHGLTPDQVVAIASHDGGKQAL | |
| | ETVQRLLPVLCQDHGLTPDQVVAIASNIGGKQALETVQALLPVLCQDHGLTPD | |
| | QVVAIASNNGGKQALETVQRLLPVLCQDHGLTPDQVVAIASNIGGKQALETVQ | |
| | ALLPVLCQDHGLTPDQVVAIASNIGGKQALETVQALLPVLCQDHGLTPDQVVAI | |
| | ASNIGGKQALETVQALLPVLCQDHGLTPDQVVAIASNNGGKQALETVQRLLPVL | |
| | CQDHGLTPDQVVAIASNIGGKQALETVQALLPVLCQDHGLTPDQVVAIASNNG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 152 |
| TCRα L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASNNGGKQALETVQRLLPVLC | |
| | QDHGLTPDQVVAIASNGGGKQALETVQRLLPVLCQDHGLTPDQVVAIASHDG | |
| | GKQALETVQRLLPVLCQDHGLTPDQVVAIASHDGGKQALETVQRLLPVLCQDH | |
| | GLTPDQVVAIASHDGGKQALETVQRLLPVLCQDHGLTPDQVVAIASNIGGKQAL | |
| | ETVQALLPVLCQDHGLTPDQVVAIASHDGGKQALETVQRLLPVLCQDHGLTPD | |
| | QVVAIASNIGGKQALETVQALLPVLCQDHGLTPDQVVAIASNNGGKQALETVQ | |
| | RLLPVLCQDHGLTPDQVVAIASNIGGKQALETVQALLPVLCQDHGLTPDQVVAI | |
| | ASNGGGKQALETVQRLLPVLCQDHGLTPDQVVAIASNIGGKQALETVQALLPV | |
| | LCQDHGLTPDQVVAIASNGGGKQALETVQRLLPVLCQDHGLTPDQVVAIASHD | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 153 |
| TCRα R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASNGGGKQALETVQRLLPVLC | |
| | QDHGLTPDQVVAIASHDGGKQALETVQRLLPVLCQDHGLTPDQVVAIASNIGG | |
| | KQALETVQALLPVLCQDHGLTPDQVVAIASNNGGKQALETVQRLLPVLCQDHG | |
| | LTPDQVVAIASHDGGKQALETVQRLLPVLCQDHGLTPDQVVAIASNGGGKQAL | |
| | ETVQRLLPVLCQDHGLTPDQWVAIASNNGGKQALETVQRLLPVLCQDHGLTPD | |
| | QVVAIASNNGGKQALETVQRLLPVLCQDHGLTPDQVVAIASNGGGKQALETVQ | |
| | RLLPVLCQDHGLTPDQVVAIASNIGGKQALETVQALLPVLCQDHGLTPDQVVAI | |
| | ASHDGGKQALETVQRLLPVLCQDHGLTPDQVVAIASNIGGKQALETVQALLPVL | |
| | CQDHGLTPDQVVAIASHDGGKQALETVQRLLPVLCQDHGLTPDQVVAIASNNG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 154 |
| PD1 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NIGGKQALETVQALLPVLCQDHGLTPDQVVAIASHDGGKQALETVQRLLPVLC | |
| | QDHGLTPDQVVAIASHDGGKQALETVQRLLPVLCQDHGLTPDQVVAIASNGG | |
| | GKQALETVQRLLPVLCQDHGLTPDQVVAIASHDGGKQALETVQRLLPVLCQDH | |
| | GLTPDQVVAIASNGGGKQALETVQRLLPVLCQDHGLTPDQVVAIASNNGGKQA | |
| | LETVQRLLPVLCQDHGLTPDQVVAIASNGGGKQALETVQRLLPVLCQDHGLTP | |
| | DQVVAIASNNGGKQALETVQRLLPVLCQDHGLTPDQVVAIASNNGGKQALETV | |
| | QRLLPVLCQDHGLTPDQVVAIASNNGGKQALETVQRLLPVLCQDHGLTPDQVV | |
| | AIASNNGGKQALETVQRLLPVLCQDHGLTPDQVVAIASHDGGKQALETVQRLL | |
| | PVLCQDHGLTPDQVVAIASHDGGKQALETVQRLLPVLCQDHGLTPDQVVAIAS | |
| | NIGGKQALETVQALLPVLCQDHGLTPQQVVAIASNGGGRPALESIVAQLSRPDP | |
| | ALAALTNDHLVALACLGGRPALDAVRRGLGDPISRSQLVKSELEEKKSELRHKL | |
| | KYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 155 |
| PD1 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASNGGGKQALETVQRLLPVLC | |
| | QDHGLTPDQVVAIASHDGGKQALETVQRLLPVLCQDHGLTPDQVVAIASNGG | |
| | GKQALETVQRLLPVLCQDHGLTPDQVVAIASNGGGKQALETVQRLLPVLCQDH | |
| | GLTPDQVVAIASNGGGKQALETVQRLLPVLCQDHGLTPDQVVAIASNNGGKQA | |
| | LETVQRLLPVLCQDHGLTPDQVVAIASNIGGKQALETVQALLPVLCQDHGLTPD | |
| | QVVAIASNGGGKQALETVQRLLPVLCQDHGLTPDQVVAIASHDGGKQALETVQ | |
| | RLLPVLCQDHGLTPDQVVAIASNGGGKQALETVQRLLPVLCQDHGLTPDQVVA | |
| | IASNNGGKQALETVQRLLPVLCQDHGLTPDQVVAIASHDGGKQALETVQRLLP | |
| | VLCQDHGLTPDQVVAIASNNGGKQALETVQRLLPVLCQDHGLTPDQVVAIASH | |
| | DGGKQALETVQRLLPVLCQDHGLTPQQVVAIASNGGGRPALESIVAQLSRPDP | |
| | ALAALTNDHLVALACLGGRPALDAVRRGLGDPISRSQLVKSELEEKKSELRHKL | |
| | KYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 156 |
| PIK3CDex | VALSQHPAALGTVAVKYQDMIAALPEATHEAIVGVGKQWSGARALEALLTVAG | |
| 8 L | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNIGGKQALETVQALLPVLC | |
| | QDHGLTPDQVVAIASNIGGKQALETVQALLPVLCQDHGLTPDQVVAIASHDGG | |
| | KQALETVQRLLPVLCQDHGLTPDQVVAIASNNGGKQALETVQRLLPVLCQDHG | |
| | LTPDQVVAIASHDGGKQALETVQRLLPVLCQDHGLTPDQVVAIASHDGGKQAL | |
| | ETVQRLLPVLCQDHGLTPDQVVAIASNNGGKQALETVQRLLPVLCQDHGLTPD | |
| | QVVAIASNIGGKQALETVQALLPVLCQDHGLTPDQVVAIASHDGGKQALETVQ | |
| | RLLPVLCQDHGLTPDQVVAIASNNGGKQALETVQRLLPVLCQDHGLTPDQVVA | |
| | IASNIGGKQALETVQALLPVLCQDHGLTPDQVVAIASNNGGKQALETVQRLLPV | |
| | LCQDHGLTPDQVVAIASHDGGKQALETVQRLLPVLCQDHGLTPDQVVAIASNN | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 157 |
| PIK3CDex | VALSQHPAALGTVAVKYQDMIAALPEATHEAIVGVGKQWSGARALEALLTVAG | |
| 8 R | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASHDGGKQALETVQRLLPVLCQDHGLTPDQVVAIASNIGG | |
| | KQALETVQALLPVLCQDHGLTPDQVVAIASHDGGKQALETVQRLLPVLCQDHG | |
| | LTPDQVVAIASHDGGKQALETVQRLLPVLCQDHGLTPDQVVAIASNGGGKQAL | |
| | ETVQRLLPVLCQDHGLTPDQVVAIASNIGGKQALETVQALLPVLCQDHGLTPD | |
| | QVVAIASNGGGKQALETVQRLLPVLCQDHGLTPDQVVAIASHDGGKQALETVQ | |
| | RLLPVLCQDHGLTPDQVVAIASHDGGKQALETVQRLLPVLCQDHGLTPDQVVA | |
| | IASHDGGKQALETVQRLLPVLCQDHGLTPDQVVAIASNIGGKQALETVQALLPV | |
| | LCQDHGLTPDQVVAIASNNGGKQALETVQRLLPVLCQDHGLTPDQVVAIASNN | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 158 |
| PIK3CDex | VALSQHPAALGTVAVKYQDMIAALPEATHEAIVGVGKQWSGARALEALLTVAG | |
| 17 L | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNIGGKQALETVQALLPVLC | |
| | QDHGLTPDQVVAIASNGGGKQALETVQRLLPVLCQDHGLTPDQVVAIASNNG | |
| | GKQALETVQRLLPVLCQDHGLTPDQVVAIASHDGGKQALETVQRLLPVLCQDH | |
| | GLTPDQVVAIASNIGGKQALETVQALLPVLCQDHGLTPDQVVAIASHDGGKQAL | |
| | ETVQRLLPVLCQDHGLTPDQVVAIASNGGGKQALETVQRLLPVLCQDHGLTPD | |
| | QVVAIASNGGGKQALETVQRLLPVLCQDHGLTPDQVVAIASNNGGKQALETVQ | |
| | RLLPVLCQDHGLTPDQVVAIASNGGGKQALETVQRLLPVLCQDHGLTPDQVVA | |
| | IASNNGGKQALETVQRLLPVLCQDHGLTPDQVVAIASHDGGKQALETVQRLLP | |
| | VLCQDHGLTPDQVVAIASNIGGKQALETVQALLPVLCQDHGLTPDQVVAIASNG | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 159 |
| PIK3CDex | VALSQHPAALGTVAVKYQDMIAALPEATHEAIVGVGKQWSGARALEALLTVAG | |
| 17 R | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNNGGKQALETVQRLLPVLC | |
| | QDHGLTPDQVVAIASNNGGKQALETVQRLLPVLCQDHGLTPDQVVAIASNIGG | |
| | KQALETVQALLPVLCQDHGLTPDQVVAIASNNGGKQALETVQRLLPVLCQDHG | |
| | LTPDQVVAIASNIGGKQALETVQALLPVLCQDHGLTPDQVVAIASNNGGKQALE | |
| | TVQRLLPVLCQDHGLTPDQVVAIASNNGGKQALETVQRLLPVLCQDHGLTPDQ | |
| | VVAIASNNGGKQALETVQRLLPVLCQDHGLTPDQVVAIASHDGGKQALETVQR | |
| | LLPVLCQDHGLTPDQVVAIASHDGGKQALETVQRLLPVLCQDHGLTPDQVVAI | |
| | ASNGGGKQALETVQRLLPVLCQDHGLTPDQVVAIASHDGGKQALETVQRLLPV | |
| | LCQDHGLTPDQVVAIASNGGGKQALETVQRLLPVLCQDHGLTPDQVVAIASNI | |
| | GGKQALETVQALLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 160 |
| S100A9 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASNIGGKQALETVQALLPVLC | |
| | QDHGLTPDQVVAIASNNGGKQALETVQRLLPVLCQDHGLTPDQVVAIASNNGG | |
| | KQALETVQRLLPVLCQDHGLTPDQVVAIASNNGGKQALETVQRLLPVLCQDHG | |
| | LTPDQVVAIASNNGGKQALETVQRLLPVLCQDHGLTPDQVVAIASHDGGKQAL | |
| | ETVQRLLPVLCQDHGLTPDQVVAIASHDGGKQALETVQRLLPVLCQDHGLTPD | |
| | QVVAIASHDGGKQALETVQRLLPVLCQDHGLTPDQVVAIASNGGGKQALETVQ | |
| | RLLPVLCQDHGLTPDQVVAIASNNGGKQALETVQRLLPVLCQDHGLTPDQVVA | |
| | IASNIGGKQALETVQALLPVLCQDHGLTPDQVVAIASHDGGKQALETVQRLLPV | |
| | LCQDHGLTPDQVVAIASNIGGKQALETVQALLPVLCQDHGLTPDQVVAIASNNG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 161 |
| S100A9 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASNGGGKQALETVQRLLPVLCQDHGLTPDQVVAIASNNG | |
| | GKQALETVQRLLPVLCQDHGLTPDQVVAIASHDGGKQALETVQRLLPVLCQDH | |
| | GLTPDQVVAIASHDGGKQALETVQRLLPVLCQDHGLTPDQVVAIASNGGGKQA | |
| | LETVQRLLPVLCQDHGLTPDQVVAIASNNGGKQALETVQRLLPVLCQDHGLTP | |
| | DQVVAIASHDGGKQALETVQRLLPVLCQDHGLTPDQVVAIASHDGGKQALETV | |
| | QRLLPVLCQDHGLTPDQVVAIASNGGGKQALETVQRLLPVLCQDHGLTPDQVV | |
| | AIASNNGGKQALETVQRLLPVLCQDHGLTPDQVVAIASNIGGKQALETVQALLP | |
| | VLCQDHGLTPDQVVAIASNNGGKQALETVQRLLPVLCQDHGLTPDQVVAIASN | |
| | NGGKQALETVQRLLPVLCQDHGLTPQQVVAIASNGGGRPALESIVAQLSRPDP | |
| | ALAALTNDHLVALACLGGRPALDAVRRGLGDPISRSQLVKSELEEKKSELRHKL | |
| | KYVPHEYIELIEIARNSTQDRILEMKVMEFFMKVYGYRGKHLGGSRKPDGAIYT | |
| | VGSPIDYGVIVDTKAYSGGYNLPIGQADEMQRYVEENQTRNKHINPNEWWKV | |
| | YPSSVTEFKFLFVSGHFKGNYKAQLTRLNHITNCNGAVLSVEELLIGGEMIKAG | |
| | TLTLEEVRRKFNNGEINFAAD | |
| TALE V2 | MGDPKKKRKVIDIADLRTLGYSQQQQEKIKPKVRSTVAQHHEALVGHGFTHAHI | 162 |
| AAVS1 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASHDGGKQALETVQRLLPVLC | |
| | QDHGLTPDQVVAIASHDGGKQALETVQRLLPVLCQDHGLTPDQVVAIASHDGG | |
| | KQALETVQRLLPVLCQDHGLTPDQVVAIASNGGGKQALETVQRLLPVLCQDHG | |
| | LTPDQVVAIASHDGGKQALETVQRLLPVLCQDHGLTPDQVVAIASHDGGKQAL | |
| | ETVQRLLPVLCQDHGLTPDQVVAIASNIGGKQALETVQALLPVLCQDHGLTPD | |
| | QVVAIASHDGGKQALETVQRLLPVLCQDHGLTPDQVVAIASHDGGKQALETVQ | |
| | RLLPVLCQDHGLTPDQVVAIASHDGGKQALETVQRLLPVLCQDHGLTPDQVVA | |
| | IASHDGGKQALETVQRLLPVLCQDHGLTPDQVVAIASNIGGKQALETVQALLPV | |
| | LCQDHGLTPDQVVAIASHDGGKQALETVQRLLPVLCQDHGLTPDQVVAIASNI | |
| | GGKQALETVQALLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 163 |
| AAVS1 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NNGGKQALETVQRLLPVLCQDHGLTPDQVVAIASNGGGKQALETVQRLLPVLC | |
| | QDHGLTPDQVVAIASHDGGKQALETVQRLLPVLCQDHGLTPDQVVAIASNIGG | |
| | KQALETVQALLPVLCQDHGLTPDQVVAIASHDGGKQALETVQRLLPVLCQDHG | |
| | LTPDQVVAIASHDGGKQALETVQRLLPVLCQDHGLTPDQVVAIASNIGGKQALE | |
| | TVQALLPVLCQDHGLTPDQVVAIASNIGGKQALETVQALLPVLCQDHGLTPDQV | |
| | VAIASNGGGKQALETVQRLLPVLCQDHGLTPDQVVAIASHDGGKQALETVQRL | |
| | LPVLCQDHGLTPDQVVAIASHDGGKQALETVQRLLPVLCQDHGLTPDQVVAIA | |
| | SNGGGKQALETVQRLLPVLCQDHGLTPDQVVAIASNNGGKQALETVQRLLPVL | |
| | CQDHGLTPDQVVAIASNGGGKQALETVQRLLPVLCQDHGLTPDQWVAIASHDG | |
| | GKQALETVQRLLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHGFTHAHI | 164 |
| CD52 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASHDGGKQALETVQRLLPVLC | |
| | QDHGLTPDQVVAIASHDGGKQALETVQRLLPVLCQDHGLTPDQVVAIASNGG | |
| | GKQALETVQRLLPVLCQDHGLTPDQVVAIASHDGGKQALETVQRLLPVLCQDH | |
| | GLTPDQVVAIASHDGGKQALETVQRLLPVLCQDHGLTPDQVVAIASNGGGKQA | |
| | LETVQRLLPVLCQDHGLTPDQVVAIASNIGGKQALETVQALLPVLCQDHGLTPD | |
| | QVVAIASHDGGKQALETVQRLLPVLCQDHGLTPDQVVAIASNGGGKQALETVQ | |
| | RLLPVLCQDHGLTPDQVVAIASHDGGKQALETVQRLLPVLCQDHGLTPDQVVA | |
| | IASNIGGKQALETVQALLPVLCQDHGLTPDQVVAIASHDGGKQALETVQRLLPV | |
| | LCQDHGLTPDQVVAIASHDGGKQALETVQRLLPVLCQDHGLTPDQVVAIASNI | |
| | GGKQALETVQALLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 165 |
| CD52 R | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | NGGGKQALETVQRLLPVLCQDHGLTPDQVVAIASNNGGKQALETVQRLLPVLC | |
| | QDHGLTPDQVVAIASHDGGKQALETVQRLLPVLCQDHGLTPDQVVAIASNGG | |
| | GKQALETVQRLLPVLCQDHGLTPDQVVAIASHDGGKQALETVQRLLPVLCQDH | |
| | GLTPDQVVAIASNGGGKQALETVQRLLPVLCQDHGLTPDQVVAIASNGGGKQA | |
| | LETVQRLLPVLCQDHGLTPDQVVAIASNIGGKQALETVQALLPVLCQDHGLTPD | |
| | QVVAIASHDGGKQALETVQRLLPVLCQDHGLTPDQVVAIASHDGGKQALETVQ | |
| | RLLPVLCQDHGLTPDQVVAIASNGGGKQALETVQRLLPVLCQDHGLTPDQVVA | |
| | IASNNGGKQALETVQRLLPVLCQDHGLTPDQVVAIASNGGGKQALETVQRLLP | |
| | VLCQDHGLTPDQVVAIASNIGGKQALETVQALLPVLCQDHGLTPDQWVAIASHD | |
| | GGKQALETVQRLLPVLCQDHGLTPQQVVAIASNGGGRPALESIVAQLSRPDPA | |
| | LAALTNDHLVALACLGGRPALDAVRRGLGDPISRSQLVKSELEEKKSELRHKLK | |
| | YVPHEYIELIEIARNSTQDRILEMKVMEFFMKVYGYRGKHLGGSRKPDGAIYTV | |
| | GSPIDYGVIVDTKAYSGGYNLPIGQADEMQRYVEENQTRNKHINPNEWWKVY | |
| | PSSVTEFKFLFVSGHFKGNYKAQLTRLNHITNCNGAVLSVEELLIGGEMIKAGTL | |
| | TLEEVRRKFNNGEINFAAD | |
| TALE V2 | MGDPKKKRKVIDIADLRTLGYSQQQQEKIKPKVRSTVAQHHEALVGHGFTHAHI | 166 |
| TCRα-2 L | VALSQHPAALGTVAVKYQDMIAALPEATHEAIVGVGKQWSGARALEALLTVAG | |
| | ELRGPPLQLDTGQLLKIAKRGGVTAVEAVHAWRNALTGAPLNLTPDQVVAIAS | |
| | HDGGKQALETVQRLLPVLCQDHGLTPDQVVAIASNGGGKQALETVQRLLPVLC | |
| | QDHGLTPDQVVAIASNNGGKQALETVQRLLPVLCQDHGLTPDQVVAIASHDGG | |
| | KQALETVQRLLPVLCQDHGLTPDQVVAIASHDGGKQALETVQRLLPVLCQDHG | |
| | LTPDQVVAIASNGGGKQALETVQRLLPVLCQDHGLTPDQVVAIASNIGGKQALE | |
| | TVQALLPVLCQDHGLTPDQVVAIASNGGGKQALETVQRLLPVLCQDHGLTPDQ | |
| | VVAIASNGGGKQALETVQRLLPVLCQDHGLTPDQVVAIASHDGGKQALETVQR | |
| | LLPVLCQDHGLTPDQVVAIASNIGGKQALETVQALLPVLCQDHGLTPDQVVAIA | |
| | SHDGGKQALETVQRLLPVLCQDHGLTPDQVVAIASHDGGKQALETVQRLLPVL | |
| | CQDHGLTPDQVVAIASNNGGKQALETVQRLLPVLCQDHGLTPDQVVAIASNIG | |
| | GKQALETVQALLPVLCQDHGLTPQQVVAIASNGGGRPALESIVAQLSRPDPAL | |
| | AALTNDHLVALACLGGRPALDAVRRGLGDPISRSQLVKSELEEKKSELRHKLKY | |
| | VPHEYIELIEIARNSTQDRILEMKVMEFFMKVYGYRGKHLGGSRKPDGAIYTVG | |
| | SPIDYGVIVDTKAYSGGYNLPIGQADEMQRYVEENQTRNKHINPNEWWKVYPS | |
| | SVTEFKFLFVSGHFKGNYKAQLTRLNHITNCNGAVLSVEELLIGGEMIKAGTLTL | |
| | EEVRRKFNNGEINFAAD | |
| TALE V2 | MGDPKKKRKVIDIADLRTLGYSQQQQEKIKPKVRSTVAQHHEALVGHG | 167 |
| TCRα-2 R | FTHAHIVALSQHPAALGTVAVKYQDMIAALPEATHEAIVGVGKQWSGAR | |
| | ALEALLTVAGELRGPPLQLDTGQLLKIAKRGGVTAVEAVHAWRNALTG | |
| | APLNLTPDQVVAIASNNGGKQALETVQRLLPVLCQDHGLTPDQVVAIAS | |
| | NGGGKQALETVQRLLPVLCQDHGLTPDQVVAIASNNGGKQALETVQRL | |
| | LPVLCQDHGLTPDQVVAIASNGGGKQALETVQRLLPVLCQDHGLTPDQ | |
| | VVAIASNIGGKQALETVQALLPVLCQDHGLTPDQVVAIASNIGGKQALET | |
| | VQALLPVLCQDHGLTPDQVVAIASNIGGKQALETVQALLPVLCQDHGLT | |
| | PDQVVAIASNGGGKQALETVQRLLPVLCQDHGLTPDQVVAIASNGGGK | |
| | QALETVQRLLPVLCQDHGLTPDQVVAIASNGGGKQALETVQRLLPVLC | |
| | QDHGLTPDQVVAIASNGGGKQALETVQRLLPVLCQDHGLTPDQVVAIA | |
| | SNNGGKQALETVQRLLPVLCQDHGLTPDQVVAIASNGGGKQALETVQ | |
| | RLLPVLCQDHGLTPDQVVAIASNNGGKQALETVQRLLPVLCQDHGLTP | |
| | DQVVAIASNIGGKQALETVQALLPVLCQDHGLTPQQVVAIASNGGGRP | |
| | ALESIVAQLSRPDPALAALTNDHLVALACLGGRPALDAVRRGLGDPISR | |
| | S | |
| TALE V2 | MGDPKKKRKVIDIADLRTLGYSQQQQEKIKPKVRSTVAQHHEALVGHG | 168 |
| TGFBRII-2 | FTHAHIVALSQHPAALGTVAVKYQDMIAALPEATHEAIVGVGKQWSGAR | |
| L | ALEALLTVAGELRGPPLQLDTGQLLKIAKRGGVTAVEAVHAWRNALTG | |
| | APLNLTPDQVVAIASNGGGKQALETVQRLLPVLCQDHGLTPDQVVAIAS | |
| | NGGGKQALETVQRLLPVLCQDHGLTPDQVVAIASHDGGKQALETVQRL | |
| | LPVLCQDHGLTPDQVVAIASNGGGKQALETVQRLLPVLCQDHGLTPDQ | |
| | VVAIASNNGGKQALETVQRLLPVLCQDHGLTPDQVVAIASNNGGKQAL | |
| | ETVQRLLPVLCQDHGLTPDQVVAIASNGGGKQALETVQRLLPVLCQDH | |
| | GLTPDQVVAIASNGGGKQALETVQRLLPVLCQDHGLTPDQVVAIASNN | |
| | GGKQALETVQRLLPVLCQDHGLTPDQVVAIASNGGGKQALETVQRLLP | |
| | VLCQDHGLTPDQVVAIASHDGGKQALETVQRLLPVLCQDHGLTPDQVV | |
| | AIASNIGGKQALETVQALLPVLCQDHGLTPDQVVAIASHDGGKQALETV | |
| | QRLLPVLCQDHGLTPDQVVAIASNIGGKQALETVQALLPVLCQDHGLTP | |
| | DQVVAIASNNGGKQALETVQRLLPVLCQDHGLTPQQVVAIASNGGGRP | |
| | ALESIVAQLSRPDPALAALTNDHLVALACLGGRPALDAVRRGLGDPISR | |
| | S | |
| TALE V2 | MGDPKKKRKVIDIADLRTLGYSQQQQEKIKPKVRSTVAQHHEALVGHG | 169 |
| TGFBRII-2 | FTHAHIVALSQHPAALGTVAVKYQDMIAALPEATHEAIVGVGKQWSGAR | |
| R | ALEALLTVAGELRGPPLQLDTGQLLKIAKRGGVTAVEAVHAWRNALTG | |
| | APLNLTPDQVVAIASNNGGKQALETVQRLLPVLCQDHGLTPDQVVAIAS | |
| | NIGGKQALETVQALLPVLCQDHGLTPDQVVAIASNGGGKQALETVQRL | |
| | LPVLCQDHGLTPDQVVAIASNNGGKQALETVQRLLPVLCQDHGLTPDQ | |
| | VVAIASNGGGKQALETVQRLLPVLCQDHGLTPDQVVAIASNNGGKQAL | |
| | ETVQRLLPVLCQDHGLTPDQVVAIASNIGGKQALETVQALLPVLCQDHG | |
| | LTPDQVVAIASNNGGKQALETVQRLLPVLCQDHGLTPDQVVAIASNIGG | |
| | KQALETVQALLPVLCQDHGLTPDQVVAIASNGGGKQALETVQRLLPVL | |
| | CQDHGLTPDQVVAIASNGGGKQALETVQRLLPVLCQDHGLTPDQVVAI | |
| | ASNGGGKQALETVQRLLPVLCQDHGLTPDQVVAIASNGGGKQALETV | |
| | QRLLPVLCQDHGLTPDQVVAIASHDGGKQALETVQRLLPVLCQDHGLT | |
| | PDQVVAIASHDGGKQALETVQRLLPVLCQDHGLTPQQVVAIASNGGGR | |
| | PALESIVAQLSRPDPALAALTNDHLVALACLGGRPALDAVRRGLGDPIS | |
| | RS | |
| TALE V2 | MGDPKKKRKVIDIADLRTLGYSQQQQEKIKPKVRSTVAQHHEALVGHG | 170 |
| TGFBRII-3 | FTHAHIVALSQHPAALGTVAVKYQDMIAALPEATHEAIVGVGKQWSGAR | |
| L | ALEALLTVAGELRGPPLQLDTGQLLKIAKRGGVTAVEAVHAWRNALTG | |
| | APLNLTPDQVVAIASNGGGKQALETVQRLLPVLCQDHGLTPDQVVAIAS | |
| | NNGGKQALETVQRLLPVLCQDHGLTPDQVVAIASHDGGKQALETVQRL | |
| | LPVLCQDHGLTPDQVVAIASNGGGKQALETVQRLLPVLCQDHGLTPDQ | |
| | VVAIASHDGGKQALETVQRLLPVLCQDHGLTPDQVVAIASNIGGKQALE | |
| | TVQALLPVLCQDHGLTPDQVVAIASNGGGKQALETVQRLLPVLCQDHG | |
| | LTPDQVVAIASNNGGKQALETVQRLLPVLCQDHGLTPDQVVAIASHDG | |
| | GKQALETVQRLLPVLCQDHGLTPDQVVAIASNIGGKQALETVQALLPVL | |
| | CQDHGLTPDQVVAIASNNGGKQALETVQRLLPVLCQDHGLTPDQVVAI | |
| | ASNNGGKQALETVQRLLPVLCQDHGLTPDQVVAIASNIGGKQALETVQ | |
| | ALLPVLCQDHGLTPDQVVAIASNGGGKQALETVQRLLPVLCQDHGLTP | |
| | DQVVAIASNGGGKQALETVQRLLPVLCQDHGLTPQQVVAIASNGGGRP | |
| | ALESIVAQLSRPDPALAALTNDHLVALACLGGRPALDAVRRGLGDPISR | |
| | S | |
| TALE V2 | MGDPKKKRKVIDIADLRTLGYSQQQQEKIKPKVRSTVAQHHEALVGHG | 171 |
| TGFBRII-3 | FTHAHIVALSQHPAALGTVAVKYQDMIAALPEATHEAIVGVGKQWSGAR | |
| R | ALEALLTVAGELRGPPLQLDTGQLLKIAKRGGVTAVEAVHAWRNALTG | |
| | APLNLTPDQVVAIASHDGGKQALETVQRLLPVLCQDHGLTPDQVVAIAS | |
| | NNGGKQALETVQRLLPVLCQDHGLTPDQVVAIASNGGGKQALETVQRL | |
| | LPVLCQDHGLTPDQVVAIASHDGGKQALETVQRLLPVLCQDHGLTPDQ | |
| | VVAIASHDGGKQALETVQRLLPVLCQDHGLTPDQVVAIASNGGGKQAL | |
| | ETVQRLLPVLCQDHGLTPDQVVAIASNNGGKQALETVQRLLPVLCQDH | |
| | GLTPDQVVAIASNGGGKQALETVQRLLPVLCQDHGLTPDQVVAIASNN | |
| | GGKQALETVQRLLPVLCQDHGLTPDQVVAIASNNGGKQALETVQRLLP | |
| | VLCQDHGLTPDQVVAIASNIGGKQALETVQALLPVLCQDHGLTPDQVV | |
| | AIASHDGGKQALETVQRLLPVLCQDHGLTPDQVVAIASNNGGKQALET | |
| | VQRLLPVLCQDHGLTPDQVVAIASHDGGKQALETVQRLLPVLCQDHGL | |
| | TPDQVVAIASNNGGKQALETVQRLLPVLCQDHGLTPQQVVAIASNGGG | |
| | RPALESIVAQLSRPDPALAALTNDHLVALACLGGRPALDAVRRGLGDPI | |
| | SRS | |
| TALE V2 | MGDPKKKRKVIDIADLRTLGYSQQQQEKIKPKVRSTVAQHHEALVGHG | 172 |
| TGFBRII-4 | FTHAHIVALSQHPAALGTVAVKYQDMIAALPEATHEAIVGVGKQWSGAR | |
| L | ALEALLTVAGELRGPPLQLDTGQLLKIAKRGGVTAVEAVHAWRNALTG | |
| | APLNLTPDQVVAIASHDGGKQALETVQRLLPVLCQDHGLTPDQVVAIAS | |
| | NGGGKQALETVQRLLPVLCQDHGLTPDQVVAIASNNGGKQALETVQRL | |
| | LPVLCQDHGLTPDQVVAIASNIGGKQALETVQALLPVLCQDHGLTPDQV | |
| | VAIASNIGGKQALETVQALLPVLCQDHGLTPDQVVAIASHDGGKQALET | |
| | VQRLLPVLCQDHGLTPDQVVAIASNNGGKQALETVQRLLPVLCQDHGL | |
| | TPDQVVAIASNGGGKQALETVQRLLPVLCQDHGLTPDQVVAIASNNGG | |
| | KQALETVQRLLPVLCQDHGLTPDQVVAIASHDGGKQALETVQRLLPVL | |
| | CQDHGLTPDQVVAIASNNGGKQALETVQRLLPVLCQDHGLTPDQVVAI | |
| | ASNNGGKQALETVQRLLPVLCQDHGLTPDQVVAIASNGGGKQALETV | |
| | QRLLPVLCQDHGLTPDQVVAIASNNGGKQALETVQRLLPVLCQDHGLT | |
| | PDQVVAIASNNGGKQALETVQRLLPVLCQDHGLTPQQVVAIASNGGGR | |
| | PALESIVAQLSRPDPALAALTNDHLVALACLGGRPALDAVRRGLGDPIS | |
| | RS | |
| TALE V2 | MGDPKKKRKVIDIADLRTLGYSQQQQEKIKPKVRSTVAQHHEALVGHG | 173 |
| TGFBRII-4 | FTHAHIVALSQHPAALGTVAVKYQDMIAALPEATHEAIVGVGKQWSGAR | |
| R | ALEALLTVAGELRGPPLQLDTGQLLKIAKRGGVTAVEAVHAWRNALTG | |
| | APLNLTPDQVVAIASNNGGKQALETVQRLLPVLCQDHGLTPDQVVAIAS | |
| | NGGGKQALETVQRLLPVLCQDHGLTPDQVVAIASNNGGKQALETVQRL | |
| | LPVLCQDHGLTPDQVVAIASNGGGKQALETVQRLLPVLCQDHGLTPDQ | |
| | WVAIASNIGGKQALETVQALLPVLCQDHGLTPDQVVAIASNIGGKQALET | |
| | VQALLPVLCQDHGLTPDQVVAIASNIGGKQALETVQALLPVLCQDHGLT | |
| | PDQVVAIASNGGGKQALETVQRLLPVLCQDHGLTPDQVVAIASNGGGK | |
| | QALETVQRLLPVLCQDHGLTPDQVVAIASNGGGKQALETVQRLLPVLC | |
| | QDHGLTPDQVVAIASNGGGKQALETVQRLLPVLCQDHGLTPDQVVAIA | |
| | SNNGGKQALETVQRLLPVLCQDHGLTPDQVVAIASNGGGKQALETVQ | |
| | RLLPVLCQDHGLTPDQVVAIASNNGGKQALETVQRLLPVLCQDHGLTP | |
| | DQVVAIASNIGGKQALETVQALLPVLCQDHGLTPQQVVAIASNGGGRP | |
| | ALESIVAQLSRPDPALAALTNDHLVALACLGGRPALDAVRRGLGDPISR | |

genomic polynucleotide sequences targeted by the above TALE proteins

| | | SEQ |
|---|---|---|
| TALE-nuclease | | ID |
| designation | Target polynucleotide sequences | NO: # |
| TALE V2 CTLA4 L | TCCTAGATGATTCCATCTgcacgggcacctccAGTGGAAATCAAGTGAA | 231 |
| TALE V2 CTLA4 R | | |
| TALE V2 CISH L | TGCGCCTAGTGACCCAGcactgcctgctcctcCACCAGCCACTGCTGTA | 232 |
| TALE V2 CISH R | | |
| TALE V2 LAG3 L | TCCAGGATCTCAGCCTTctgcgaagagcagggGTCACTTGGCAGCATCA | 233 |
| TALE V2 LAG3 R | | |
| TALE V2 TGFBRII L | TCCCTATGAGGAGTATGcctcttggaagacagAGAAGGACATCTTCTCA | 234 |
| TALE V2 | | |
| TGFBRII R | | |
| TALE V2 CCR5 L | TATCAAGTGTCAAGTCCaatctatgacatcAATTATTATACATCGGA | 235 |
| TALE V2 CCR5 R | | |
| TALE V2 B2m L | TTAGCTGTGCTCGCGCTactctctctttctGGCCTGGAGGCTATCCA | 236 |
| TALE V2 B2m R | | |
| TALE V2 B2m-2 L | TCCGTGGCCTTAGCTGTgctcgcgctactcTCTCTTTCTGGCCTGGA | 237 |
| TALE V2 B2m-2 R | | |
| TALE V2 TCRa L | TTGTCCCACAGATATCCagaaccctgaccctgCCGTGTACCAGCTGAG | 238 |
| TALE V2 TCRa R | | |
| TALE V2 PD1 L | TACCTCTGTGGGGCCATctccctggcccccaaGGCGCAGATCAAAGAGA | 239 |
| TALE V2 PD1 R | | |
| TALE V2 PIK3CDex8 L | TGAACGCCGACGAGCGGatgaaggtggggcTCCTGGGATAGGTGGGA | 240 |
| TALE V2 PIK3CDex8 R | | |
| TALEV2 PIK3CDex17L | TGATGCACTTGTGCATGcggcaggaggcctacCTAGAGGCCCTCTCCCA | 241 |
| TALEV2 PIK3CDex17R | | |
| TALEV2 S100A9 L | TTAGGGGCCCTGACAGCtctccataggtggagGCCTCAGGCAGGCAGGA | 242 |
| TALE V2 S100A9 R | | |
| TALE V2 AAVS1 L | TCCCCTCCACCCCACAGtggggccactagGGACAGGATTGGTGACA | 243 |
| TALE V2 | | |
| AAVS1 R | | |
| TALE V2 CD52 L | TTCCTCCTACTCACCATcagcctcctggttatGGTACAGGTAAGAGCAA | 244 |
| TALE V2 | | |
| CD52 R | | |
| TALE V2 TCRa-2 L | TCTGCCTATTCACCGATtttgattctcaaacaAATGTGTCACAAAGTAA | 245 |
| TALE V2 TCRa-2 R | | |
| TALE V2 TGFBRII-2 L | TGTGTAAATTTTGTGATgtgagattttccaCCTGTGACAACCAGAAA | 246 |
| TALE V2 | | |
| TGFBRII-2 R | | |
| TALE V2 TGFBRII-3 L | TGATGTGAGATTTTCCAcctgtgacaaccagAAATCCTGCATGAGCAA | 247 |
| TALE V2 TGFBRII-3 R | | |
| TALE V2 TGFBRII-4 L | TCGTCCTGTGGACGCGTatcgccagcacgatCCCACCGCACGTTCAGA | 248 |
| TALE V2 TGFBRII-4 R | | |

In some preferred embodiments, the TALE-proteins of the present invention can be used by pairs, each member of this pair binding DNA close to each other, side-by-side or on opposite DNA strands, in such a way they are co-localized in the genome with the effect of directing the catalytic activity induced by the catalytic domain at a specified locus. For instance, a pair of TALE-proteins fused to the homodimerizing Fok1 nuclease domain, also referred to as “left-” and “right-” TALE-Nuclease monomers, form heterodimers that induce DNA double strand break cleavage. In such instances, the invention provides that one monomer as per the present invention can be used with another monomer that is based on a conventional TALE-Nuclease scaffold using canonical AvrBs3 sequences. Indeed, as shown in the experimental section herein, one TALE-nuclease monomer of the present invention is sufficient to have an overall effect on the heterodimeric specificity.

The present invention thus provides a number of new TALE fusion monomers based on the TALE-proteins listed in Table X, comprising such proteins fused with a nuclease or deaminase domain, for their use in genetic therapeutic modifications, in-vivo or in-vitro, as well as for the ex-vivo preparation of therapeutic cells.

According to a particular aspect, the invention provides TALE-protein monomers to introduce a genetic modification, preferably a mutation, into the CTLA4 gene locus, preferably into a target sequence comprising SEQ ID NO:231, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO:138 or SEQ ID NO: 139. In particular, the invention provides TALE-nuclease monomers, consisting of or comprising a polypeptide sequence at least 90%, preferably 95% or 99% identity with a sequence selected from SEQ ID NO: 174, and SEQ ID NO: 175.

According to a particular aspect, the invention provides TALE-protein monomers to introduce a genetic modification, preferably a mutation, into the CISH gene locus, preferably into a target sequence comprising SEQ ID NO:232, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO:140 or SEQ ID NO:141. In particular, the invention provides TALE-nuclease monomers, consisting of or comprising a polypeptide sequence at least 90%, preferably 95% or 99% identity with a sequence selected from SEQ ID NO: 176, and SEQ ID NO: 177.

According to a particular aspect, the invention provides TALE-protein monomers to introduce a genetic modification, preferably a mutation, into the LAG3 gene locus, preferably into a target sequence comprising SEQ ID NO:233, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 142 or SEQ ID NO:143. In particular, the invention provides TALE-nuclease monomers, consisting of or comprising a polypeptide sequence at least 90%, preferably 95% or 99% identity with a sequence selected from SEQ ID NO: 178, and SEQ ID NO: 179.

According to a particular aspect, the invention provides TALE-protein monomers to introduce a genetic modification, preferably a mutation, into the TGFBRII gene locus, preferably into a target sequence comprising SEQ ID NO:234, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 144 or SEQ ID NO:145. In particular, the invention provides TALE-nuclease monomers, consisting of or comprising a polypeptide sequence at least 90%, preferably 95% or 99% identity with a sequence selected from SEQ ID NO: 180, and SEQ ID NO: 181.

According to a particular aspect, the invention provides TALE-protein monomers to introduce a genetic modification, preferably a mutation, into the CCR5 gene locus, preferably into a target sequence comprising SEQ ID NO:235, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 146 or SEQ ID NO: 147. In particular, the invention provides TALE-nuclease monomers, consisting of or comprising a polypeptide sequence at least 90%, preferably 95% or 99% identity with a sequence selected from SEQ ID NO: 182, and SEQ ID NO: 183.

According to a particular aspect, the invention provides TALE-protein monomers to introduce a genetic modification, preferably a mutation, into the B2m gene locus, preferably into a target sequence comprising SEQ ID NO:236 or SEQ ID NO:237, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO: 2, 3 or 4. Said TALE-protein preferably comprises SEQ ID NO: 148, SEQ ID NO: 149, SEQ ID NO: 150 or SEQ ID NO: 151. In particular, the invention provides TALE-nuclease monomers, consisting of or comprising a polypeptide sequence at least 90%, preferably 95% or 99% identity with a sequence selected from SEQ ID NO: 184, SEQ ID NO: 185, SEQ ID NO: 186 and SEQ ID NO: 187.

According to a particular aspect, the invention provides TALE-protein monomers to introduce a genetic modification, preferably a mutation, into the TCRalpha gene locus, preferably into a target sequence comprising SEQ ID NO:238, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 152 or SEQ ID NO: 153. In particular, the invention provides TALE-nuclease monomers, consisting of or comprising a polypeptide sequence at least 90%, preferably 95% or 99% identity with a sequence selected from SEQ ID NO: 188, and SEQ ID NO: 189

According to a particular aspect, the invention provides TALE-protein monomers to introduce a genetic modification, preferably a mutation, into the PD1 gene locus, preferably into a target sequence comprising SEQ ID NO:239, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 154 or SEQ ID NO: 155. In particular, the invention provides TALE-nuclease monomers, consisting of or comprising a polypeptide sequence at least 90%, preferably 95% or 99% identity with a sequence selected from SEQ ID NO: 190, and SEQ ID NO: 191.

According to a particular aspect, the invention provides TALE-protein monomers to introduce a genetic modification, preferably a mutation, into the PIK3CDex8 gene locus, preferably into a target sequence comprising SEQ ID NO:240, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO: 2, 3 or 4. Said TALE-protein preferably comprises SEQ ID NO: 156 or SEQ ID NO: 157. In particular, the invention provides TALE-nuclease monomers, consisting of or comprising a polypeptide sequence at least 90%, preferably 95% or 99% identity with a sequence selected from SEQ ID NO: 192, and SEQ ID NO: 193.

According to a particular aspect, the invention provides TALE-protein monomers to introduce a genetic modification, preferably a mutation, into the PIK3CDex17 gene locus, preferably into a target sequence comprising SEQ ID NO:241, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO: 2, 3 or 4. Said TALE-protein preferably comprises SEQ ID NO: 158 or SEQ ID NO: 159. In particular, the invention provides TALE-nuclease monomers, consisting of or comprising a polypeptide sequence at least 90%, preferably 95% or 99% identity with a sequence selected from SEQ ID NO: 194, and SEQ ID NO: 195.

According to a particular aspect, the invention provides TALE-protein monomers to introduce a genetic modification, preferably a mutation, into the S100A9 gene locus, preferably into a target sequence comprising SEQ ID NO:242, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 160 or SEQ ID NO: 161. In particular, the invention provides TALE-nuclease monomers, consisting of or comprising a polypeptide sequence at least 90%, preferably 95% or 99% identity with a sequence selected from SEQ ID NO: 196, and SEQ ID NO: 197.

According to a particular aspect, the invention provides TALE-protein monomers to introduce a genetic modification, preferably a mutation, into the AAVS1 gene locus, preferably into a target sequence comprising SEQ ID NO:243, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 162 or SEQ ID NO: 163. In particular, the invention provides TALE-nuclease monomers, consisting of or comprising a polypeptide sequence at least 90%, preferably 95% or 99% identity with a sequence selected from SEQ ID NO: 198, and SEQ ID NO: 199.

According to a particular aspect, the invention provides TALE-protein monomers to introduce a genetic modification, preferably a mutation, into the CD52 gene locus, preferably into a target sequence comprising SEQ ID NO:244, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 164 or SEQ ID NO: 165. In particular, the invention provides TALE-nuclease monomers, consisting of or comprising a polypeptide sequence at least 90%, preferably 95% or 99% identity with a sequence selected from SEQ ID NO:200, and SEQ ID NO: 201.

According to a particular aspect, the invention provides TALE-protein monomers to introduce a genetic modification, preferably a mutation, into the TCR alpha gene locus, preferably into a target sequence comprising SEQ ID NO:245, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. Said TALE-protein preferably comprises SEQ ID NO: 166 or SEQ ID NO: 167. In particular, the invention provides TALE-nuclease monomers, consisting of or comprising a polypeptide sequence at least 90%, preferably 95% or 99% identity with a sequence selected from SEQ ID NO:202, and SEQ ID NO: 203.

According to a particular aspect, the invention provides TALE-protein monomers to introduce a genetic modification, preferably a mutation, into the TGFBRII gene locus, preferably into a target sequence comprising SEQ ID NO:246, 247 or 248, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO: 2, 3 or 4. Said TALE-protein preferably comprises SEQ ID NO: 168, SEQ ID NO: 169, SEQ ID NO: 170, SEQ ID NO: 171, SEQ ID NO: 172 or SEQ ID NO: 173. In particular, the invention provides TALE-nuclease monomers, consisting of or comprising a polypeptide sequence at least 90%, preferably 95% or 99% identity with a sequence respectively selected from SEQ ID NO:204, SEQ ID NO:205, SEQ ID NO:206, SEQ ID NO:207, SEQ ID NO:208 and SEQ ID NO:209.

According to a particular aspect, the invention provides TALE-protein monomers to introduce a genetic modification, preferably a mutation, into the TIGIT gene locus, preferably into a target sequence comprising or consisting of SEQ ID NO:289, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO: 2, 3 or 4. In particular, the invention provides TALE-nuclease monomers, consisting of or comprising a polypeptide sequence having at least 90%, preferably 95% or 99% identity with SEQ ID NO: 269 and/or SEQ ID NO:270.

According to a particular aspect, the invention provides TALE-protein monomers to introduce a genetic modification, preferably a mutation, into the CISH gene locus, preferably into a target sequence comprising or consisting of SEQ ID NO:290, 291 and/or 292, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. In particular, the invention provides with TALE-nuclease monomers, consisting of or comprising a polypeptide sequence having at least 90%, preferably 95% or 99% identity with SEQ ID NO:271, SEQ ID NO:272, SEQ ID NO:273, SEQ ID NO:274, SEQ ID NO:275 and/or SEQ ID NO:276.

According to a particular aspect, the invention provides TALE-protein monomers to introduce a genetic modification, preferably a mutation, into the CD38 gene locus, preferably into a target sequence comprising or consisting of SEQ ID NO:293 and/or SEQ ID NO:294, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. In particular, the invention provides with TALE-nuclease monomers, consisting of or comprising a polypeptide sequence having at least 90%, preferably 95% or 99% identity with SEQ ID NO:277, SEQ ID NO:278, SEQ ID NO:279, and/or SEQ ID NO:280.

According to a particular aspect, the invention provides TALE-protein monomers to introduce a genetic modification, preferably a mutation, into the IgH gene locus, preferably into a target sequence comprising or consisting of SEQ ID NO:295 and/or SEQ ID NO:296, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. In particular, the invention provides with TALE-nuclease monomers, consisting of or comprising a polypeptide sequence having at least 90%, preferably 95% or 99% identity with SEQ ID NO:281, SEQ ID NO:282, SEQ ID NO:283, and/or SEQ ID NO:284.

According to a particular aspect, the invention provides TALE-protein monomers to introduce a genetic modification, preferably a mutation, into the GADPH gene locus, preferably into a target sequence comprising or consisting of SEQ ID NO:297 and/or SEQ ID NO:298, wherein said TALE protein comprises (1) a TALE binding domain comprising at least 3, preferably at least 4, 5, 6, 7, 8, 9, 10, 11, 12, 13, 14 or 15 repeats comprising SEQ ID NO:5 to 11 and (2) a C-terminal polypeptide sequence from 40 to 80 residues comprising a sequence having at least 85% identity with SEQ ID NO:2, 3 or 4. In particular, the invention provides with TALE-nuclease monomers, consisting of or comprising a polypeptide sequence having at least 90%, preferably 95% or 99% identity with SEQ ID NO:285, SEQ ID NO:286, SEQ ID NO:287, and/or SEQ ID NO: 288.By “mutation” is meant herein any change of one or more nucleotide in a characterized polynucleotide sequence (wild type), generally into a genomic sequence into a cell, said change including the deletion or substitution of said nucleotide (or base pair), the deletion insertion, integration or translocation of a polynucleotide fragment, oligonucleotide, or exogenous sequence, such as a transgene. Such mutation generally leads to a correction, loss or gain of function by the cell, which genome is modified.

### Development of TALE-Transcription Factors

By following the previous teachings, the TALE proteins according to the invention can also be fused to desired transcriptional activator and repressor protein domains to create specific trans-activator or repressor reagents in view of controlling endogenous gene expression.

As an example, artificial transcription factors can be obtained by fusion of a TALE protein of the present invention with VP64 or the 16 amino acid peptide VP16 (SEQ ID NO: 120) from herpes simplex virus as described by Miller J.C., et al. [A TALE nuclease architecture for efficient genome editing (2011) Nat Biotechnol 29 (2): 143-148].

To accomplish repression of a gene, the TALE proteins of the present invention can be fused for example with Kruppel-associated box (KRAB), Sid4, or EAR-repression domain (SRDX), which have been previously reported as being strong pleiotropic repressors [Cong L, et al. (2012) Comprehensive interrogation of natural TALE DNA-binding modules and transcriptional repressor domains. Nat Commun 3 (1): 968].

### Development of TALE-Base Editors

By following the previous teachings, the TALE proteins according to the invention can also be fused to desired base editors.

The term “base editor” as used herein, refers to a catalytic domain capable of making a modification to a base (e.g., A, T, C, G, or U) within a nucleic acid sequence that converts one base to another (e.g., A to G, A to C, A to T, C to T C to G, C to A, G to A, G to C, G to T, T to A, T to C, T to G). Adenine and cytosine base editors catalytic domains are described, for instance, in Rees & Liu [Base editing: precision chemistry on the genome and transcriptome of living cells (2018) Nat. Rev. Genet. 19 (12): 770-788].

Catalytic base editors can include cytidine deaminase that convert target C/G to T/A and adenine base editors that convert target A/T to G/C. Preferred cytosine deaminase can be cytosine deaminase 1 (pCDM) or Activation-induced cytidine deaminase (AICDA). Preferred adenosine deaminase can be TadA (SEQ ID NO: 121) or its variant TadA7.10 as described by Jeong, Y. K., et al. [Adenine base editor engineering reduces editing of bystander cytosines (2021) Nat. Biotechnol. https://doi.org/10.1038/s41587-021-00943]. Different members of Apolipoprotein B mRNA editing enzyme (APOBEC) family can be used convert cytidines to thymidines, such as the murine rAPOBEC1 and the human APOBEC3G (SEQ ID NO: 130) as developed by Lee et al. [Single C-to-T substitution using engineered APOBEC3G-nCas9 base editors with minimum genome- and transcriptome-wide off-target effects (2020) Science Advances. 6 (29)].

In preferred embodiments, base editor catalytic domain converts a C to T (cytidine deaminase) that catalyzes the chemical reaction “cytosine+H2O->uracil+NH3” or “5-methyl-cytosine+H2O->thymine+NH3.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function.

In some embodiments, the TALE-base editors according to the present invention can comprise a domain that inhibits uracil glycosylase referred to as “UGI”, and/or a nuclear localization signal. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a canonical UGI as set forth in SEQ ID NO: 136. In some embodiments, the UGI proteins provided herein include fragments of UGI and proteins homologous to a UGI or a UGI fragment comprising an amino acid sequence that comprises at least 60%, at least 65%, at least 70%, at least 75%, at least 80%, at least 85%, at least 90%, at least 95%, at least 96%, at least 97%, at least 98%, at least 99%, of the amino acid sequence as set forth in SEQ ID NO: 136. TALE base editors according to the present invention comprising UGI are useful to improve the specificity of base editing performed at a predetermined locus.

In some embodiments, the base editor catalytic domain is a double-stranded DNA deaminase (“DddA”) to precisely install nucleotide changes and/or correct pathogenic mutations, rather than destroying DNA with double-strand breaks (DSBs). In preferred embodiments, DddAtox is generally split into inactive fragments which can be separately delivered to a target deamination site on separate TALE-base editor constructs that will co-localize each fragment of the DddA on site, such as on either side of a target edit site, where they reform a functional DddA that is capable deaminating a target site on the double-stranded DNA molecule. In certain embodiments, the programmable DNA binding proteins can be engineered to comprise one or more mitochondrial localization signals (MLS), in such a way that the DddA domains become translocated into the mitochondria, thereby providing a means by which to conduct base editing directly on the mitochondrial genome.

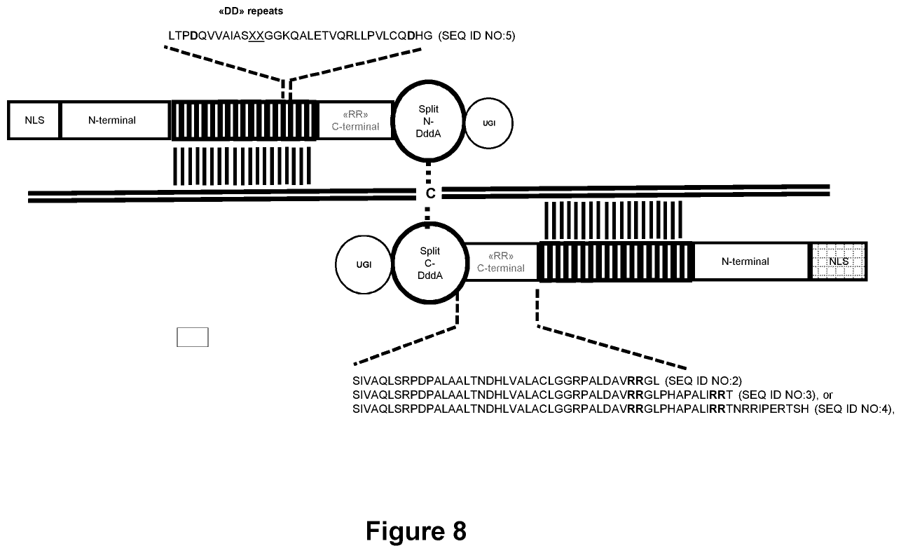

Fragments of the DddA can be formed by truncating DddAtox (i.e., dividing or splitting the DddA protein) at specified amino acid residues, such as one selected from the group comprising: 62, 71, 73, 84, 94, 108, 110, 122, 135, 138, 148, and 155. In preferred embodiments, the truncation of DddA occurs at residue 148. In certain embodiments, the DddA can be separated into two fragments by dividing the DddA at one of these split sites to form N-terminal and C-terminal portion of the DddA, which may be referred to as “DddA-N half′ and “DddA-C half.”. According to preferred embodiments said “DddA-N half” and “DddA-C half.” comprise an amino acid sequence that respectively share at least 60%, at least 65%, at least 70%, at least 75%, at least 80%, at least 85%, at least 90%, at least 95%, at least 96%, at least 97%, at least 98%, at least 99%, with the amino acid sequence SEQ ID NO. 134 and SEQ ID NO: 135. As shown in FIG. 8, two TALE proteins acting by pairs respectively comprising N and C-DddA halves can be used to co-localize and induce on-site nucleobase change.

TALE-base editors of the present invention can also be used by pairs, each member comprising different but complementary catalytic domains in view of obtaining a given base editing reaction at one precise locus.

### Development of TALE-Transposase or Integrase

By following the previous teachings, the TALE proteins according to the invention can also be fused to a transposase or an integrase in order to perform site-directed integration of transgenes into the genome.

As an example, the TALE protein according to the invention can be fused to the PiggyBac transposase as described for instance by Owens, J. B. et al. [Transcription activator like effector (TALE)-directed piggyBac transposition in human cells (2013) N.A.R. 41 (19): 9197-9207]. The PiggyBac transposase is autonomously functional in such system so that a co-transfected transposon is able to integrate into any genomic location specified by the TALE protein. This system can permanently introduce large cassettes (>100 kb) encoding numerous components such as multiple transgenes, insulators and inducible or endogenous promoters and allows to potentially target integrations to nearly any genomic region. This system is especially worth in situations where safe single-targeted insertions need to be verified ex vivo, and cells be amplified and re-infused into patients. Targeted transposition could be used to intentionally disrupt endogenous coding regions or to direct insertions to user-defined genomic safe harbours to protect the cargo from unknown chromosomal position effects and to circumvent accidental mutation of target cells.

### Development of TALE-Proteins to Edit the Epigenome

Still following the previous teachings, TALE-protein fusions can be made by fusion with catalytic domains that can modulate the expression of a gene without altering the DNA sequence, especially by remodelling chromatin.

In this regard, TALE proteins as per the present invention can be fused to methyltransferase obtain histone methylation and/or with a p300 effector domain that enhances histone acetyltransferase.

Conversely, TALE protein can be fused to the catalytic domain thymidine DNA glycosylase (TDG) to abolish the DNA methylation and induce gene expression. Unwanted DNA methylations are associated with many neurodegenerative diseases. TALE protein could be fused to TET domain (ten-eleven translocation methylcytosine dioxygenase 2) as an example, for targeting epigenetically silenced cancer gene (ICAM-1) and induce its expression in cancerous cells. TET1 can also be used used in the treatment of many diseases like diabetes (inducing β cell replication) and cancer (inhibiting cell proliferation) [Ou K., et al. (2019) Targeted demethylation at the CDKN1C/p57 locus induces human B cell replication. J Clin Invest 129 (1): 209-214].

The present invention encompasses the polynucleotides, in particular DNA or RNA encoding the polypeptides and proteins previously described, as well as any intermediary products involved in any aspects and steps of the methods described herein. These polynucleotides may be included in vectors, more particularly plasmids or virus, in view of being expressed in prokaryotic or eukaryotic cells.

The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, especially AAV6 vectors, parvovirus (e.g. adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

As per the present invention, the TALE proteins or polynucleotide encoding thereof, especially mRNA, can also be loaded into nanoparticles for their effective delivery into cells. A variety of nanoparticles are described in the art to target particular tissues of cell types [Friedman A.D. et al. (2013) The Smart Targeting of Nanoparticles Curr Pharm Des. 19 (35): 6315-6329]. Preferred nanoparticles are positively charged nanoparticles, such as silica based nanoparticles or LNP (Lipid nanomolar nanoparticles) as described in the art with other types of nucleases [Conway, A. et al. (2019) Non-viral Delivery of Zinc Finger Nuclease mRNA Enables Highly Efficient In Vivo Genome Editing of Multiple Therapeutic Gene Targets, Molecular Therapy 27 (4): 866-877].

Alternatively, the polynucleotides encoding the present TALE proteins of the present invention, especially under mRNA form can be electroporated directly into blood cells by electroporation, by using for instance the steps described in WO2013176915 on pages 29 and 30 incorporated herein by reference.

The present invention also relates to methods for use of said polypeptides polynucleotides and proteins previously described for various applications ranging from targeted nucleic acid cleavage to targeted gene regulation. In genome engineering experiments, the efficiency of the nuclease fusion proteins as referred to in the present patent application, e.g. their ability to induce a desired event (Homologous gene targeting, targeted mutagenesis, sequence removal or excision, base editing) at a locus, depends on several parameters, including the specific activity of the nuclease, probably the accessibility of the target, and the efficacy and outcome of the repair pathway(s) resulting in the desired event (homologous repair for gene targeting, NHEJ pathways for targeted mutagenesis), which can be assessed by standard techniques known in the art. The present invention more particularly relates to a method for modifying the genetic material of a cell within or adjacent to a nucleic acid target sequence by using one TALE fusion protein of the present invention. The double strand breaks caused by a TALE-nuclease, for instance, are commonly repaired through non-homologous end joining (NHEJ). NHEJ comprises at least two different processes. Mechanisms involve rejoining of what remains of the two DNA ends through direct re-ligation or via the so-called microhomology-mediated end joining. Repair via non-homologous end joining (NHEJ) often results in small insertions or deletions and can be used for the creation of specific gene knockouts.

Other aspects of the present disclosure relate to pharmaceutical compositions comprising any of the various components of the TALE proteins obtainable by the methods of the present invention (e.g., TALE-nuclease, TALE-deaminase, TALE-transcriptase, TALE-methylase, TALE-transposase . . . ).

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, the pharmaceutical composition are provided as reagents to correct genetic deficiencies, which can be used in vivo or ex-vivo, especially in gene therapy.

In preferred embodiments, the TALE proteins of the present invention are used to genetically modify blood cells ex-vivo, especially immune cells such as T-cells and NK cells, preferably primary cells to produce therapeutic cells for immunotherapy.

In some embodiments, the pharmaceutical composition is formulated in accordance with routine procedures as a composition adapted for intravenous or subcutaneous administration to a subject (e.g., a human). In some embodiments, pharmaceutical composition for administration by injection are solutions in sterile isotonic aqueous buffer. Where necessary, the pharmaceutical can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6:1438-47). Positively charged lipids such as N-[I-(2,3-dioleoyloxi) propyl]-N, N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising foir example: (a) a container containing a compound of the invention in lyophilized form; and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use or sale for human administration.

### EXAMPLES

### Example 1: Methods

### TALE-Nuclease Heterodimers Construction

Mutations in the DNA targeting modules, linker domain or Fokl domain were introduced using de novo gene synthesis (Integrated DNA Technologies or Genescript) and TALE-nuclease monomers were assembled using standard molecular biology technics such as enzymatic restriction digestion, ligation, bacterial transformation. Integrity of all sequences was assessed by Sanger sequencing.

TALE-Nuclease Fusion mRNA Production

Plasmids encoding the TALE-nuclease heterodimers are transformed into XL1 Blue competent bacteria according to standard molecular biology procedures. At least two colonies were picked as miniprep cultures from the agarose plate and DNA extracted via QIAprep 96 plus Miniprep kit according to the manufacturer's protocol (Qiagen). Sequence validated plasmids were linearized using standard molecular biology techniques and purified using the Nucleospin Gel and PCR Clean-up kit (Macherey-Nagel). mRNA was produced using the HiScribe T7 ARCA mRNA Kit according to the manufacturer's protocol (NEB) and purified with Mag-Bind Total Pure NGS magnetic beads (Omega) on the KingFisher Flex System (Thermo Fisher Scientific) as per the manufacturer's instructions.

### Cells

Cryopreserved human PBMCs were cultured in X-vivo-15 media (Lonza Group), containing IL-2 (Miltenyi Biotech,), and human serum AB (Seralab). Dynabeads Human T-Activator CD3/CD28 for T Cell Expansion and Activation (Thermo Fisher Scientific) were used, according to the provider's protocol, to activate T-cells for 3 days before passage in fresh media.

### TALE-Nuclease Electroporation

Two different protocols have been used alternatively in the different sets of experiments:
(A) Four days following activation, human T lymphocytes were transfected by electroporation using an AgilePulse MAX system (Harvard Apparatus): cells were pelleted and resuspended in cytoporation medium T at >28×10⁶ cells/ml. 5×10⁶ cells were mixed with 10 μg total of indicated TALE-nuclease mRNA (5 μg each of the left and right monomers) into a 0.4 cm cuvette. In parallel, mock transfections (no mRNA) were performed. The electroporation consisted of two 0.1 ms pulses at 800 V followed by four 0.2 ms pulses at 130V. Following electroporation, cells were split in half and diluted into 1.2 mL fresh warm culture medium in separate plates and incubated at 30° C./5% CO₂ overnight. Cell were passaged in complete medium and kept at 37° C./5% CO₂ for 2 days.
(B) Alternatively, four days following activation, human T lymphocytes were transfected by electroporation (program code EO 115) using an Lonza 4D Nucleofector (Lonza): cells were pelleted, washed with PBS, and resuspended using the P3 Primary Cell 4D-Nucleofector X Kit (Lonza) at >50×10⁶ cells/ml. 1×10⁶ cells were mixed with 1-3 μg total mRNA (0.5-1.5 μg each of the left and right monomers) into the 96-well Shuttle add-on for the 4D Nucleofector system (Lonza). In parallel, mock transfections (no mRNA) were performed. Following electroporation, cells were transferred in a 96w or 48w culture plate containing warm fresh warm culture medium incubated at 30° C./5% CO₂ overnight. Cell were passaged in complete medium and kept at 37° C./5% CO₂ for 2 days.

Cells were pelleted by centrifugation and genomic DNA was extracted using the Mag-Bind Blood & Tissue DNA HDQ 96 Kit (Omega) on the KingFisher Flex System (Thermo Fisher Scientific) as per the manufacturer's instructions.

Targeted PCR of the endogenous locus was performed using Phusion High Fidelity PCR Master Mix with HF Buffer (NEB) for amplification of a ˜300 bp region surrounding the TALE-nuclease cut on-PCR products were purified using the Mag-Bind Total Pure NGS magnetic beads (Omega) on the KingFisher Flex System (Thermo Fisher Scientific) as per the manufacturer's instructions. Amplicons were further analyzed by deep-sequencing (Illumina).

### TALE-Nuclease Cleavage Specificity Evaluation

Oligo capture assay was adapted from (Tsai et al., GUIDE-seq paper) and carried out on the Fluent Automation Workstation liquid handler robot (Tecan).

TALE-nucleases were co-electroporated with unspecific oligonucleotides amplifiable by PCR, cells were transferred in a 96w or 48w culture plate containing warm fresh warm culture medium incubated at 30° C./5% CO₂ overnight. Cell were passaged in complete medium and kept at 37° C./5% CO₂ for 2 days. Cells were pelleted by centrifugation and genomic DNA was extracted using the Mag-Bind Blood & Tissue DNA HDQ 96 Kit (Omega) on the KingFisher Flex System (Thermo Fisher Scientific) as per the manufacturer's instructions.

Final libraries were further analyzed by deep-sequencing (Illumina).

### Example 2: Effect of Mutations in the C-Terminal Domain

Starting from canonical TALE-nuclease fusions (SEQ ID: 210 and SEQ ID NO:211) composing heterodimeric TALE-Fok1 nuclease (V0) as described by Christian et al. [Targeting DNA Double-Strand Breaks with TAL Effector Nucleases (2010) Genetics 186:757-761] by targeting a 49 base pair sequence into the human CS1 gene (SEQ ID NO:228), two sets of substitutions were introduced in the C-terminal sequence between the DNA binding core and the Fokl catalytic head at positions K37 and K38 (relative to the canonical AvrBs3 C40 SEQ ID NO: 109) (i) two histidine (HH, V0.1; SEQ ID NO:212 and SEQ ID NO:213) and (ii) two arginine (RR, V0.2; SEQ ID NO:214 and SEQ ID NO:215).

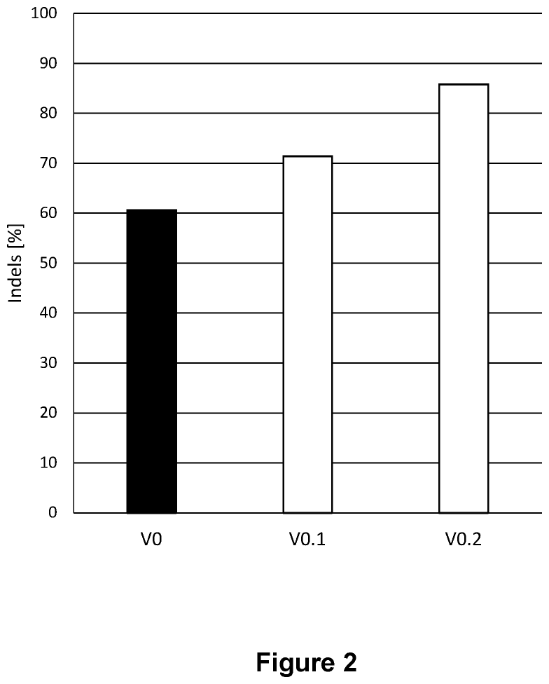

Activity of the resulting TALE-nuclease containing either monomers with mutation or both was assessed in primary T-cells as described in example 1. The presence of a single heterodimer with the above substitutions HH and RR respectively led to higher activity as demonstrated with Indel frequencies (FIG. 2). The TALE-nuclease activity was also improved in presence of both RR mutated TALE-nuclease heterodimers.

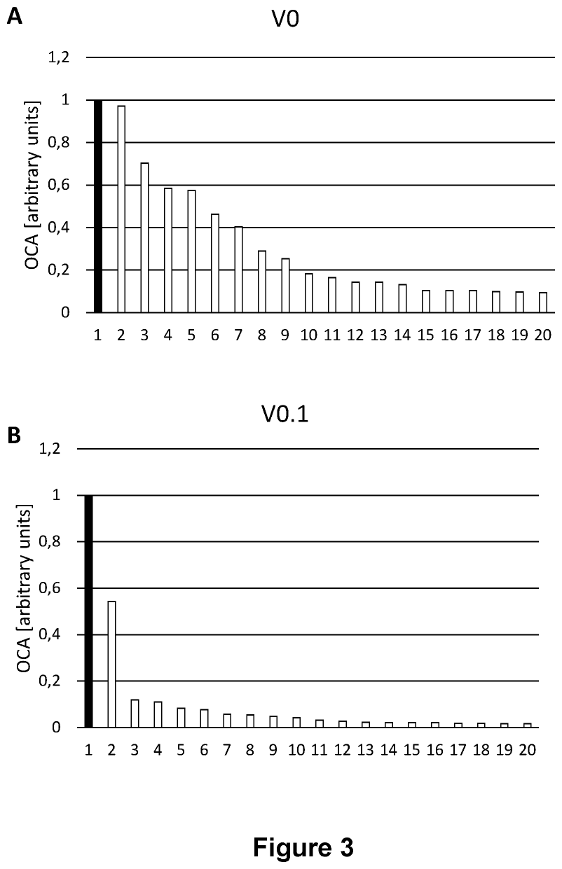

Importantly, while activity was enhanced in the singe mutated TALE monomers with HH, the genome wide specificity profile, as assessed by the oligo capture assay, was also improved (FIG. 3).

### Example 3: Effect of Amino Acid Changes in the DNA Targeting Repeats and in the C-Terminal Domain

Starting from the same canonical TALE-nuclease heterodimers (SEQ ID NO:210 and SEQ ID NO: 211) targeting the 49 base pair target sequence in CS1 (SEQ ID NO:228), a set of substitutions was introduced in the DNA binding repeats (SEQ ID NO:24 to 27) leading to V1 heterodimeric TALE-nuclease (SEQ ID NO:216 and SEQ ID: 217).

Starting from V1 arginine (R) mutations were further introduced in positions K37 and K38 into the C-terminal sequence, leading to V1.2 (SEQ ID NO:218 and SEQ ID NO:219).

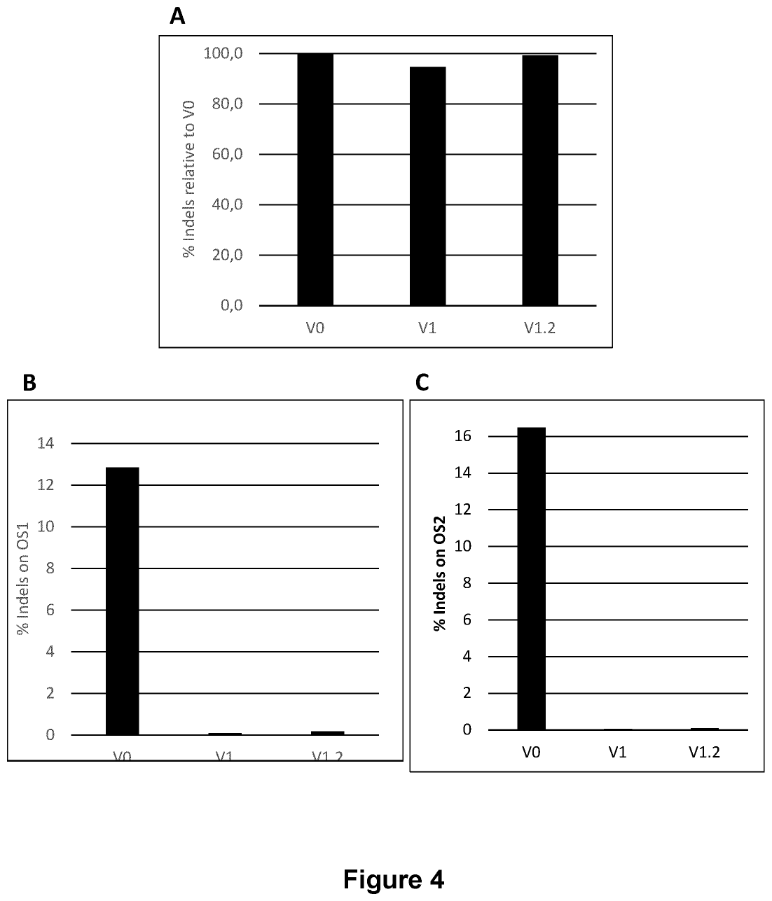

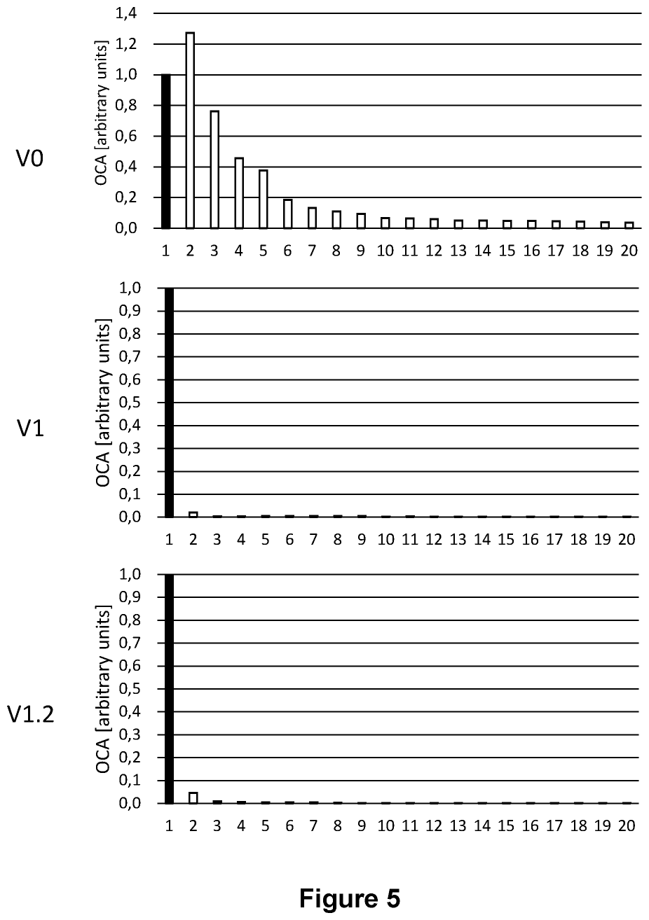

Activity of the resulting TALE-nucleases V1 and V1.2 and the original TALEN (V0) was assessed in primary T-cells as described in example 1. An activity matching the V0 TALEN was recovered in using the V1.2 TALE-nuclease, as demonstrated with Indel frequencies (FIG. 4). Indels frequencies was further assessed on two off-site targets, OS1 and OS2 (SEQ ID NO:229 and SEQ ID NO:230). FIG. 5 shows that Indels frequencies on both targets were reduced to background by using both V1 and V1.2 TALE-nucleases.

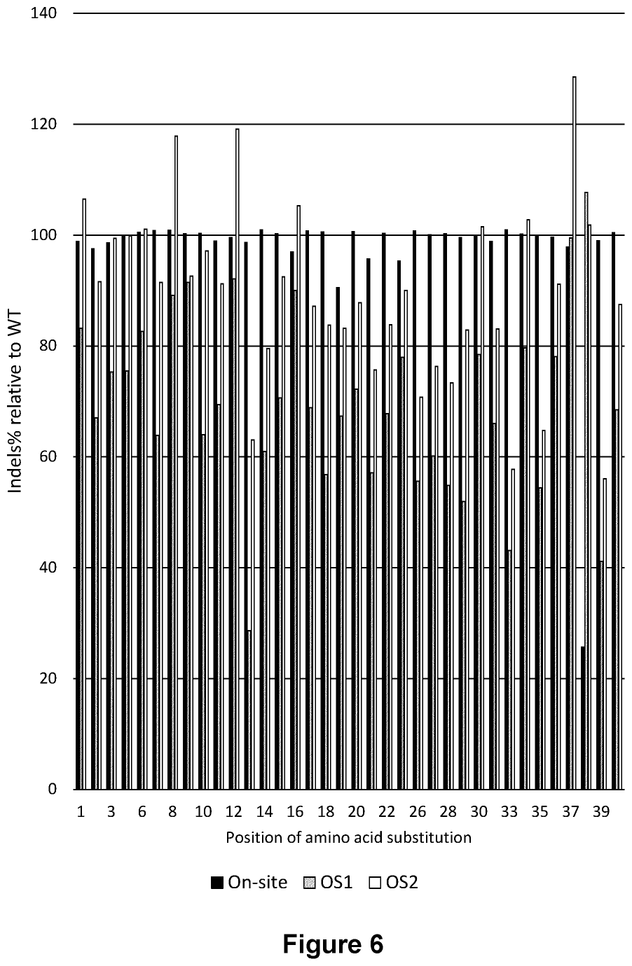

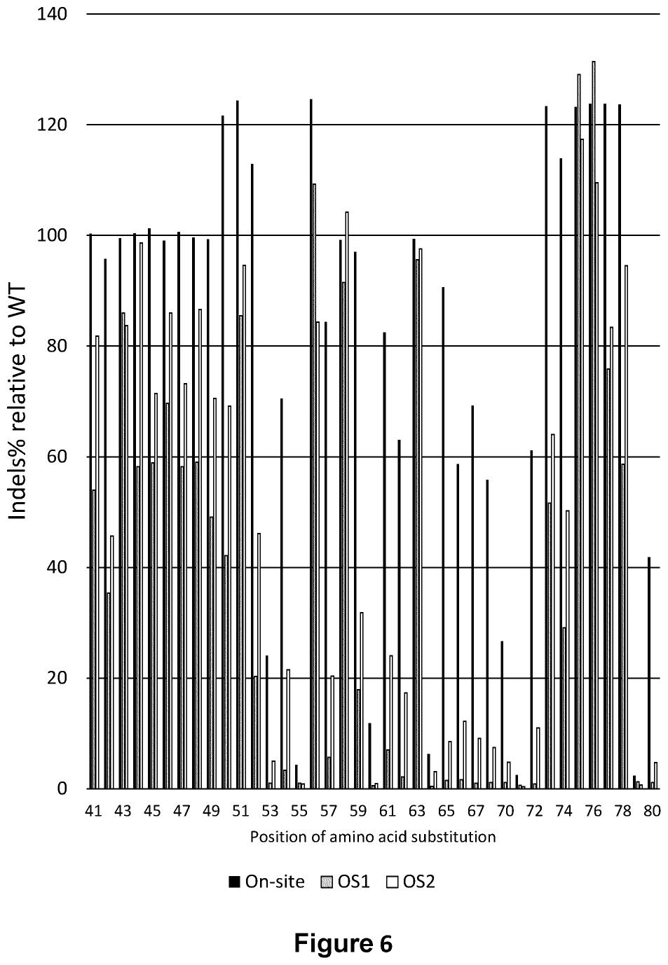

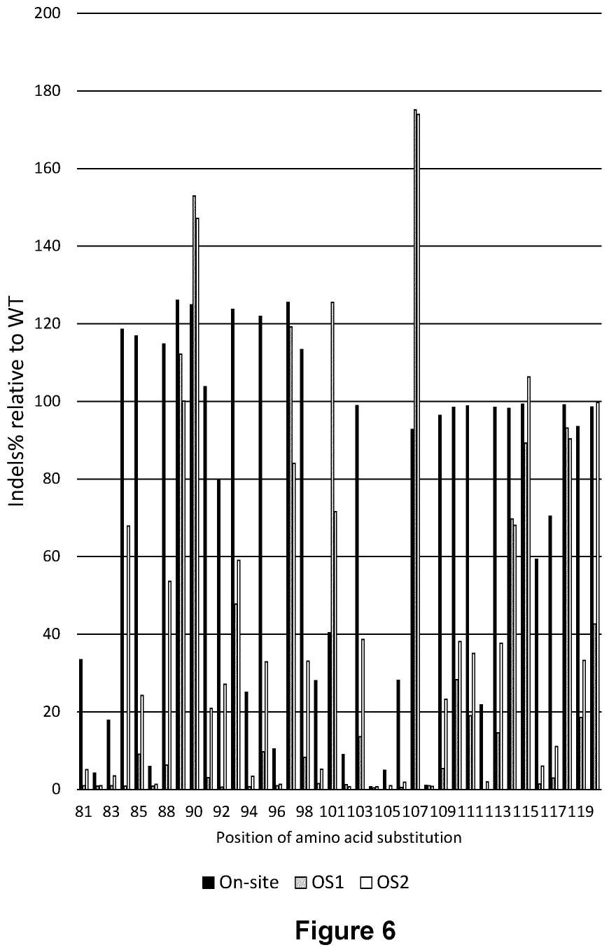

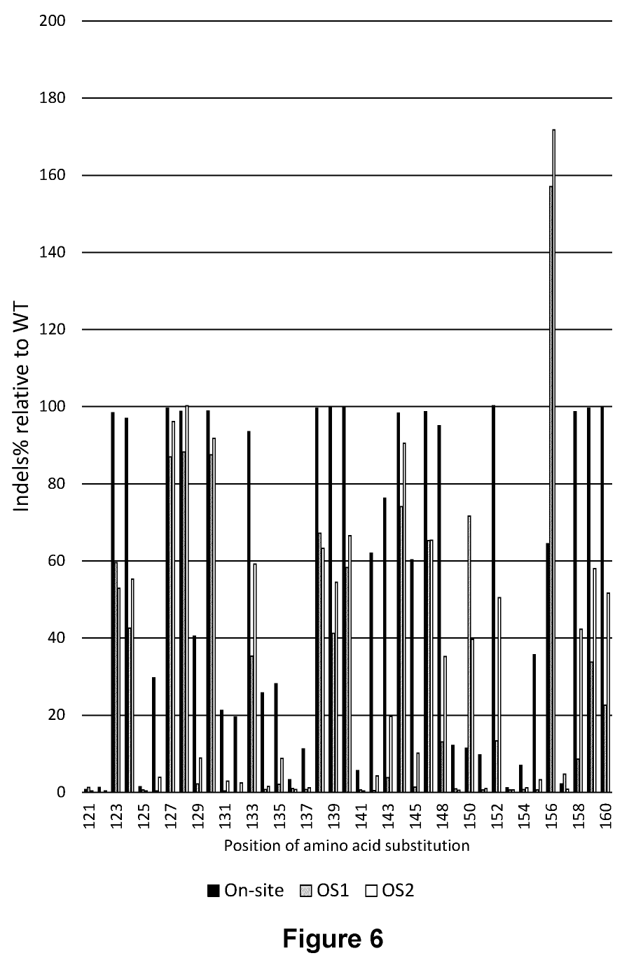

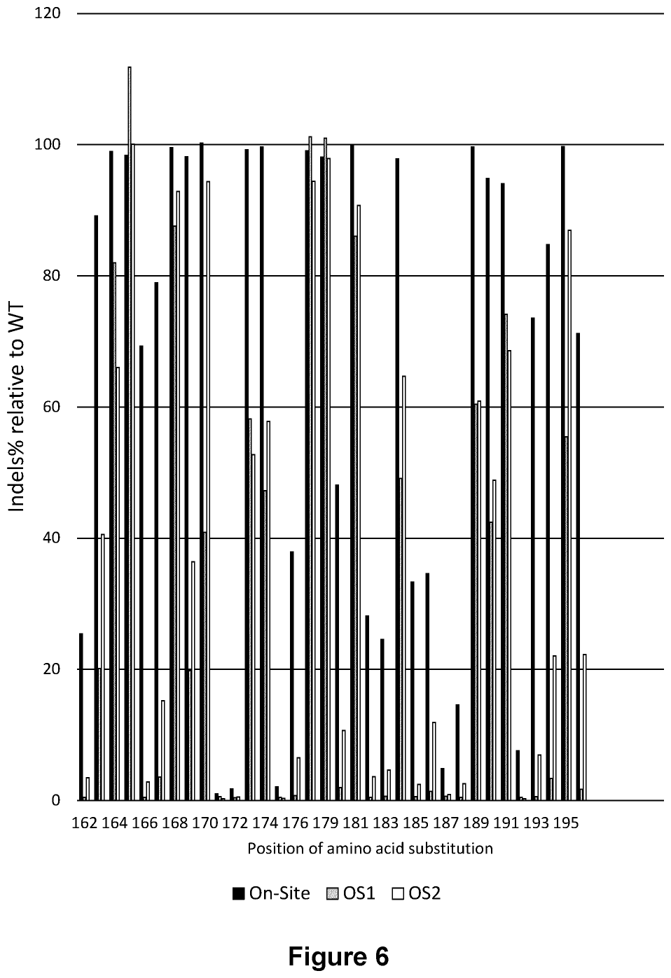

Finally, the genome wide specificity profile, as assessed by the oligo capture assay, was improved in by using V1 and V1.2 heterodimer structures when compared to V0 (FIG. 6) with activity detected only on the specific CS1 original target sequence.

### Example 4: Effect of Amino Acid Changes in the Fokl Catalytic Head

A library of monomers of V0 structure (SEQ ID NO:210) was created by substituting, one by one, each amino acid of the wild type Fokl catalytic domain (SEQ ID NO: 109) by an alanine.

The TALE-nuclease activity resulting from the heterodimer formed by each of the substituted V0 monomers resulting and of the other untouched monomer of SEQ ID NO:210 was assessed by indels formation on the “on-site” target (SEQ ID NO:228) and the 2 “off-sites” targets, OS1 and OS2 (SEQ ID NO:229 and SEQ ID NO:230).

Indels detection, at the “on-site” and “off-sites”, for each variants of the library was normalized to the Indels obtained with the wild type Fok1 (pCLS32855 and pCLS31911) (SEQ ID NO: 210 and SEQ ID NO:211) (FIG. 6).

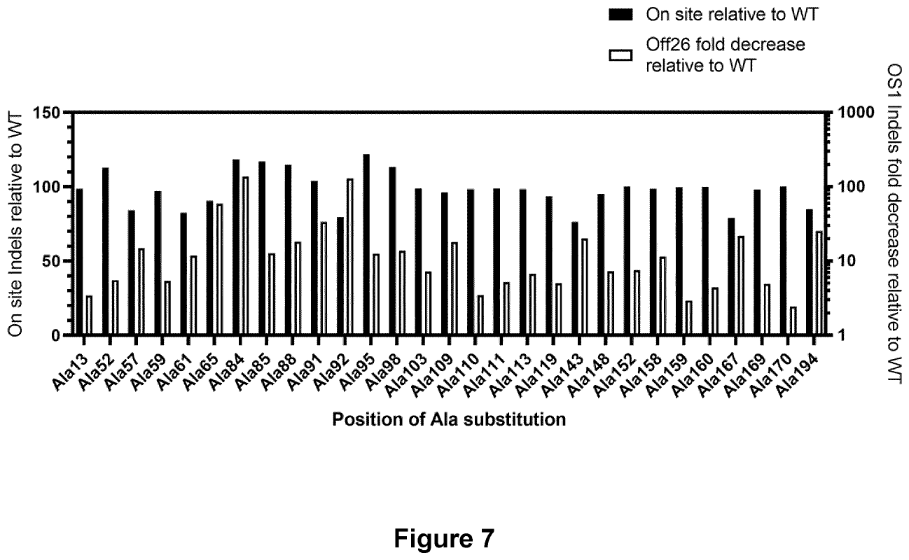

As shown in FIG. 7, a number of substitutions into the Fok1 catalytic domain have been found to correlate with decreased indels formation into the predicted off target OS1, while maintaining a substantial nuclease activity above 70% with respect to the wild type Fok1 sequence. These alanine substitutions into SEQ ID NO: 109 concerned amino acid positions 13, 52, 57, 59, 61, 65, 84, 85, 88, 91, 92, 95, 98, 103, 109, 110, 111, 113, 119, 143, 148, 152, 158, 159, 160, 167, 169, 170 and 194. Some substitutions have been found to decrease indels formation, while maintaining the full nuclease activity, such as the substitutions introduced at positions 84, 85, 88, 95, 98, 91, 103, 109, 148, 152 and 158, and even led to an increase of nuclease activity (more than 100% activity) at positions 84, 88 and 91.

### Example 5: TALE-Base Editor to Introduce a Non-Sense Mutation into the CD52 Gene TALE-Base Editors Heterodimers Construction

Polynucleotides sequences have been designed to target and convert 1 or more nucleobase C into T into the CD52 target sequences SEQ ID NO:249 to 252, also referred to in Table 6, in view of expressing the heterodimer structures that are illustrated in FIG. 8 aiming at disrupting a splice site or introducing a mutation into those target sequences and inactivate the surface presentation of CD52 in primary T-cells.

One polynucleotide sequence encodes a first monomer comprising a TALE protein fused to a NLS at its N-terminus and to the N-split DddA deaminase+UGI at its C-terminus (respectively SEQ ID NO:220, SEQ ID NO:222, SEQ ID NO:224 and SEQ ID NO:226);

The other polynucleotide sequence encodes a second monomer comprising a TALE protein fused to a NLS at its N-terminus and to the C-split DddA deaminase+UGI at its C-terminus (respectively SEQ ID NO:221, SEQ ID NO:223, SEQ ID NO:225 and SEQ ID NO:227).

The polynucleotide sequences of the above TALE proteins were assembled using standard molecular biology technics using enzymatic restriction digestion, ligation and bacterial transformation. Integrity of all the polynucleotide sequences was assessed by Sanger sequencing.

The polynucleotide sequences encoding the above monomers have been cloned into plasmids for production in adequate bacteria such as XL1-Blue.

TALE-Nuclease Fusion mRNA Production

Plasmids encoding the TALE-nuclease heterodimers are transformed into XL1 Blue competent bacteria according to standard molecular biology procedures. At least two colonies were picked as miniprep cultures from the agarose plate and DNA extracted via QIAprep 96 plus Miniprep kit according to the manufacturer's protocol (Qiagen). Sequence validated plasmids were linearized using standard molecular biology techniques and purified using the Nucleospin Gel and PCR Clean-up kit (Macherey-Nagel). mRNA was produced using the HiScribe T7 ARCA mRNA Kit according to the manufacturer's protocol (NEB) and purified with Mag-Bind Total Pure NGS magnetic beads (Omega) on the KingFisher Flex System (Thermo Fisher Scientific) as per the manufacturer's instructions.

### Cells

Cryopreserved human PBMCs were cultured in X-vivo-15 media (Lonza Group), containing IL-2 (Miltenyi Biotech,), and human serum AB (Seralab). Dynabeads Human T-Activator CD3/CD28 for T Cell Expansion and Activation (Thermo Fisher Scientific) were used, according to the provider's protocol, to activate T-cells for 3 days before passage in fresh media.

### TALE-Base Editors Nuclease Electroporation

Four days following activation, human T lymphocytes were transfected by electroporation using an AgilePulse MAX system (Harvard Apparatus): cells were pelleted and resuspended in cytoporation medium T at >28×10⁶ cells/ml. 5×10⁶ cells were mixed with 10 μg total of indicated TALE-nuclease mRNA (5 μg each of the left and right monomers) into a 0.4 cm cuvette. In parallel, mock transfections (no mRNA) were performed. The electroporation consisted of two 0.1 ms pulses at 800 V followed by four 0.2 ms pulses at 130V. Following electroporation, cells were split in half and diluted into 1.2 mL fresh warm culture medium in separate plates and incubated at 30° C./5% CO₂ overnight. Cell were passaged in complete medium and kept at 37° C./5% CO₂ for 2 days.

Cells were pelleted by centrifugation and genomic DNA was extracted using the Mag-Bind Blood & Tissue DNA HDQ 96 Kit (Omega) on the KingFisher Flex System (Thermo Fisher Scientific) as per the manufacturer's instructions.

Targeted PCR of the endogenous locus was performed using Phusion High Fidelity PCR Master Mix with HF Buffer (NEB) for amplification of a ˜300 bp region spanning the CD52 target sequence (SEQ ID NO:249, 250, 251 and 252) as per the manufacturer's instructions. Amplicons were further analyzed by deep-sequencing (Illumina) for detection of mutational events (nucleobase conversion).

### Example 6: Improved Specificity of TALE-Nuclease Targeting TGFBRII Gene Sequence

### Off Target Analysis of the TALE-Nucleases Targeting TGFBRII

A “classical” version (V0) of TALEN monomers targeting TGFBRII gene sequence (SEQ ID NO: 234) was compared with an improved TALEN monomer version V1.2 as per the present invention comprising the tandem DD-RR mutations and tested for its specificity by oligo capture assay.

mRNAs encoding the “classical” TALE-nucleases (V0) and DD-RR (V1.2) monomers targeting TGFBRII gene sequence SEQ ID NO:234 were by using the mMessage mMachine T7 Ultra kit (Life Technologies) and purified with RNeasy columns (Qiagen) and eluted in water or cytoporation medium T (Harvard Apparatus) as described in Poirot et al. [Cancer Res (2015) 75 (18): 3853-3864].

The heterodimeric pairs V0—V0, V0-V1.2 and V1.2-V1.2 were respectively co-electroporated with unspecific oligonucleotides amplifiable by PCR in order to perform oligo capture assay analysis at predicted off-site genomic locations. These predicted off-site locations had been previously identified with respect to the V0-VO TALEN monomers.

Left and right monomers polypeptide sequences are provided in Table 5 below.

Cryopreserved human PBMCs were cultured in X-vivo-15 media (Lonza Group), containing IL-2 (Miltenyi Biotech,), and human serum AB (Seralab). Dynabeads Human T-Activator CD3/CD28 for T Cell Expansion and Activation (Thermo Fisher Scientific) were used, according to the provider's protocol, to activate T-cells. Six days post activation, T lymphocytes were electroporated using an AgilePulse MAX system (Harvard Apparatus) with the different TALE-nuclease versions targeting the same TGFBRII target sequence (SEQ ID NO: 234). The TALE-nuclease used were either containing no mutation (V0-V0) corresponding to SEQ ID NO: 267 and SEQ ID NO:268, or were comprising one half TALE-nuclease containing the DD-RR mutations (V1.2-V0) corresponding to SEQ ID NO: 181 and SEQ ID NO:268, or finally both half TALE-nuclease containing the DD-RR mutations (V1.2-V1.2) corresponding to SEQ ID NO: 181 and SEQ ID NO: 180. T-cells were pelleted and resuspended in cytoporation medium T and 10⁶ cells were electroporated with 0.5 μg of each indicated half TALE-nuclease. The electroporation consisted of two 0.1 ms pulses at 800 V followed by four 0.2 ms pulses at 130V. Following electroporation, cells were incubated at 30° C./5% CO₂ for 18 hours. Cell were passaged in complete medium and kept at 37° C./5% CO₂ for 1 day and expended for 18 days. Genomic DNA (gDNA) was extracted using Qiagen DNeasy blood & tissue kit according to manufacturer's protocol. 200 ng of gDNA were used for High fidelity PCR amplification of the on- and off-site loci using primers listed in Table 6. Amplicons were further analyzed by deep-sequencing (Illumina) to identify potential insertions at the predetermined off-site loci.

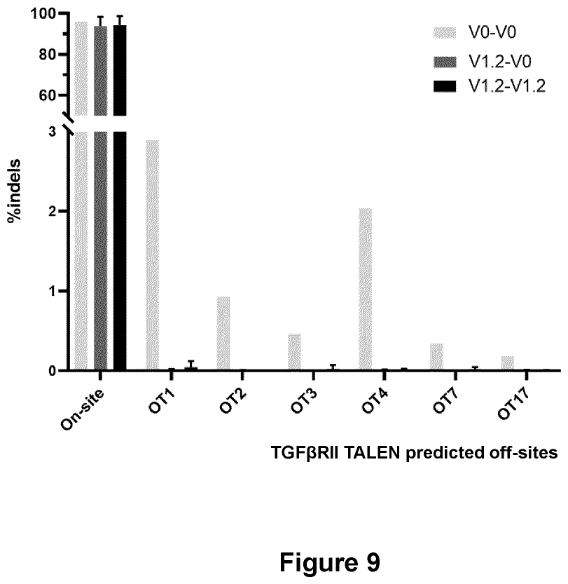

As shown in the graphic representations of FIG. 9, the percentage of indels induced by each TALE-nuclease on the on-site were equivalent, whereas the indels induced at the different analyzed off-target sites (OT #) were no longer detected in the T-cells transfected with at least one V1.2 TALE-nuclease monomer comprising the tandem DD-RR mutations, thereby demonstrating an improved specificity of the TALEN monomers according to the present invention.

### Example 7: TALE-Nucleases Designed Under V1.2 Targeting TIGIT, CISH, CD38, IgH and GADPH Gene Sequences

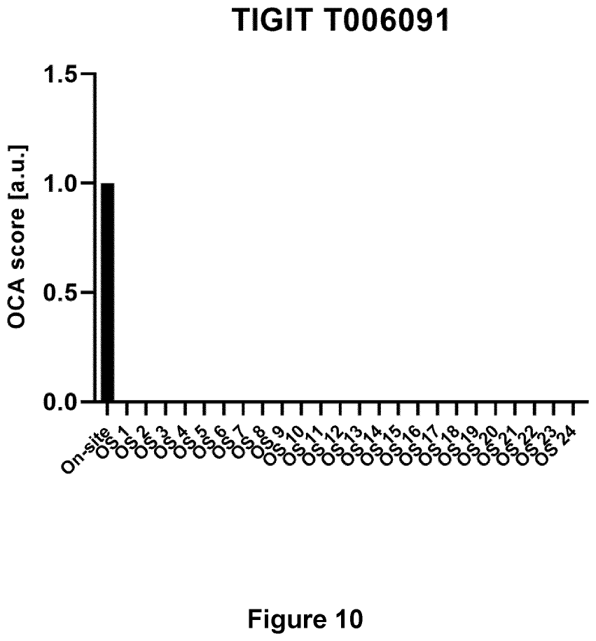

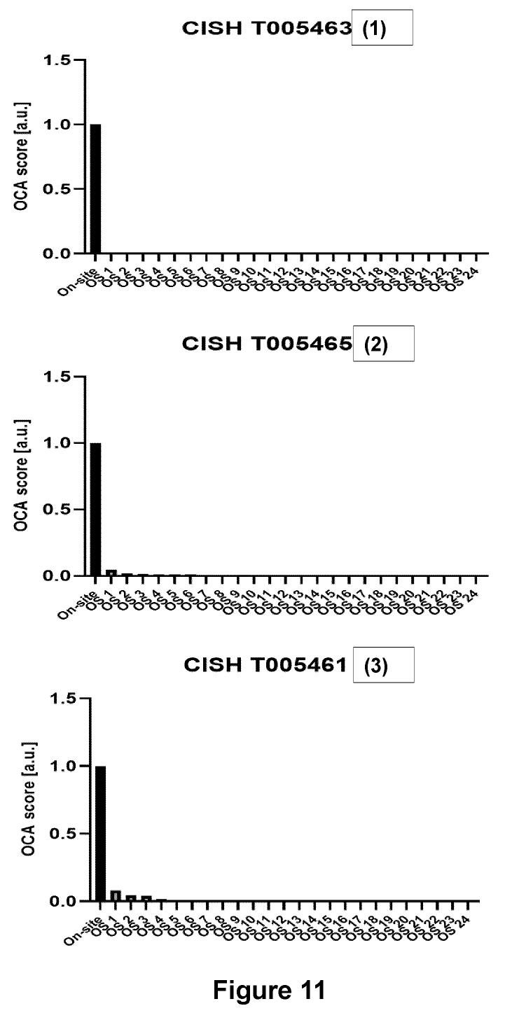

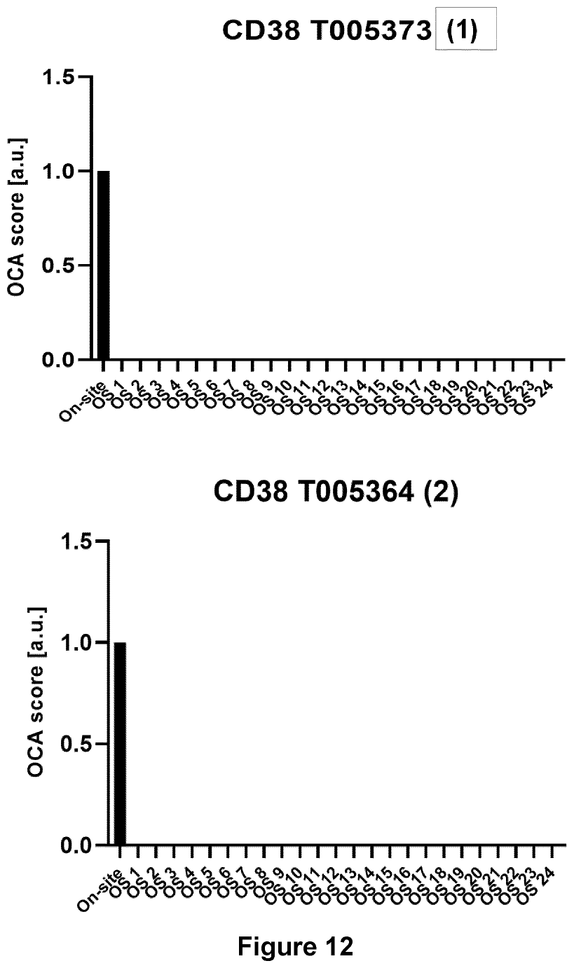

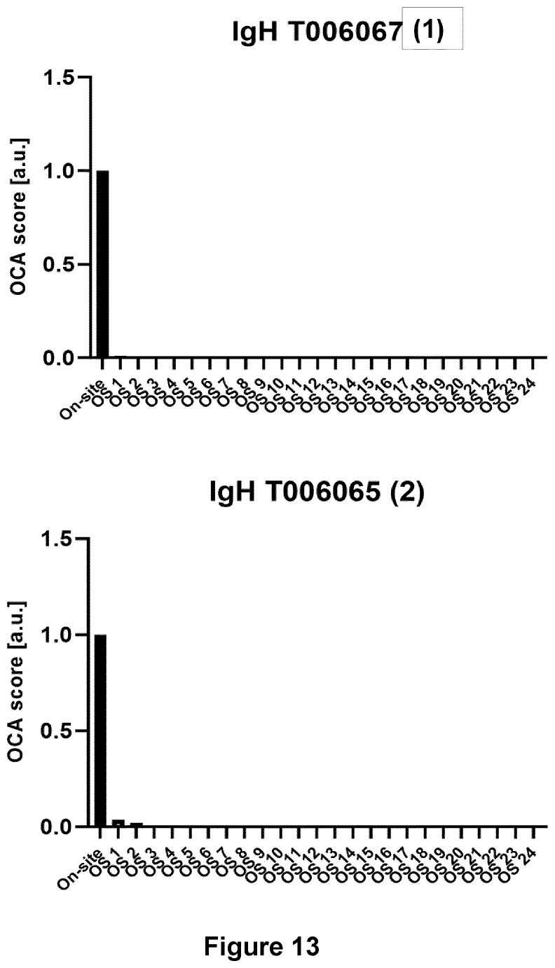

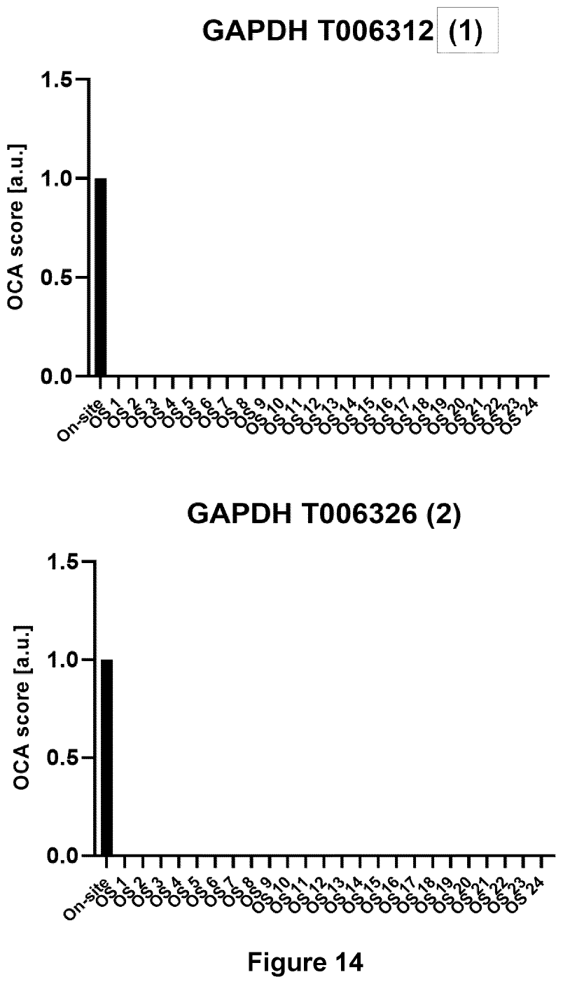

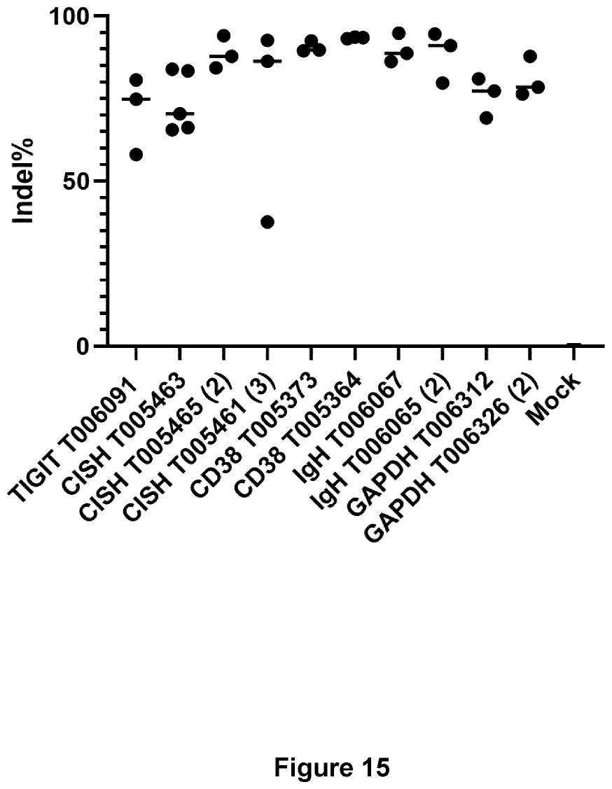

TALE-nucleases have been designed and tested for their specificity as described in Example 1 in order to target genomic sequences th respective TIGIT, CISH, CD38, IgH, and GADPH human genes. The polynucleotide sequences targeted in these genes are presented in Table 6. The polypeptide sequences of the left and right TALE-nuclease heterodimers are provided in Table 5. Results of the oligo capture assays for each TALEN V2/target sequence couples are displayed in FIGS. 10 to 14, showing high specificity of the TALE scaffolds of the present invention and constantly high activit (% activity higher than 50%, mostly above 70% shown in FIG. 15).

TABLE 5
Polypeptide sequences used in the Examples

| | | |
|---|---|---|
| | | SEQ |
| TALE | | ID |
| protein | | NO: |
| designation | Polypeptide sequence | # |

EXAMPLE 2

| | | |
|---|---|---|
| CS1 TALEN | MGDPKKKRKVIDIADLRTLGYSQQQQEKIK | 210 |
| Left V0 | PKVRSTVAQHHEALVGHGFTHAHIVALSQH | |
| pCLS32855 | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPQQVVAIASNGGGKQALETVQRLLPVLCQ | |
| | AHGLTPEQVVAIASHDGGKQALETVQRLLP | |
| | VLCQAHGLTPEQVVAIASHDGGKQALETVQ | |
| | RLLPVLCQAHGLTPEQVVAIASNIGGKQAL | |
| | ETVQALLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | EQVVAIASNIGGKQALETVQALLPVLCQAH | |
| | GLTPQQVVAIASNNGGKQALETVQRLLPVL | |
| | CQAHGLTPEQVVAIASHDGGKQALETVQRL | |
| | LPVLCQAHGLTPEQVVAIASNIGGKQALET | |
| | VQALLPVLCQAHGLTPEQVVAIASNIGGKQ | |
| | ALETVQALLPVLCQAHGLTPQQVVAIASNG | |
| | GGKQALETVQRLLPVLCQAHGLTPEQVVAI | |
| | ASNIGGKQALETVQALLPVLCQAHGLTPQQ | |
| | VVAIASNGGGKQALETVQRLLPVLCQAHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 211 |
| right V0 | PKVRSTVAQHHEALVGHGFTHAHIVALSQH | |
| pCLS31911 | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPEQVVAIASNIGGKQALETVQALLPVLCQ | |
| | AHGLTPQQVVAIASNNGGKQALETVQRLLP | |
| | VLCQAHGLTPEQVVAIASNIGGKQALETVQ | |
| | ALLPVLCQAHGLTPQQVVAIASNGGGKQAL | |
| | ETVQRLLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | QQVVAIASNNGGKQALETVQRLLPVLCQAH | |
| | GLTPQQVVAIASNNGGKQALETVQRLLPVL | |
| | CQAHGLTPQQVVAIASNGGGKQALETVQRL | |
| | LPVLCQAHGLTPQQVVAIASNNGGKQALET | |
| | VQRLLPVLCQAHGLTPEQVVAIASNIGGKQ | |
| | ALETVQALLPVLCQAHGLTPQQVVAIASNN | |
| | GGKQALETVQRLLPVLCQAHGLTPQQVVAI | |
| | ASNNGGKQALETVQRLLPVLCQAHGLTPEQ | |
| | VVAIASHDGGKQALETVQRLLPVLCQAHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 212 |
| V0.1 HH | PKVRSTVAQHHEALVGHGFTHAHIVALSQH | |
| left | PAALGTVAVKYQDMIAALPEATHEAIVGVG | |
| pCLS33633 | KQWSGARALEALLTVAGELRGPPLQLDTGQ | |
| | LLKIAKRGGVTAVEAVHAWRNALTGAPLNL | |
| | TPQQVVAIASNGGGKQALETVQRLLPVLCQ | |
| | AHGLTPEQVVAIASHDGGKQALETVQRLLP | |
| | VLCQAHGLTPEQVVAIASHDGGKQALETVQ | |
| | RLLPVLCQAHGLTPEQVVAIASNIGGKQAL | |
| | ETVQALLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | EQVVAIASNIGGKQALETVQALLPVLCQAH | |
| | GLTPQQVVAIASNNGGKQALETVQRLLPVL | |
| | CQAHGLTPEQVVAIASHDGGKQALETVQRL | |
| | LPVLCQAHGLTPEQVVAIASNIGGKQALET | |
| | VQALLPVLCQAHGLTPEQVVAIASNIGGKQ | |
| | ALETVQALLPVLCQAHGLTPQQVVAIASNG | |
| | GGKQALETVQRLLPVLCQAHGLTPEQVVAI | |
| | ASNIGGKQALETVQALLPVLCQAHGLTPQQ | |
| | VVAIASNGGGKQALETVQRLLPVLCQAHGL | |
| | TPQQVVAIASNGGGRPALESIVAQLSRPDP | |
| | ALAALTNDHLVALACLGGRPALDAVHHGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRINHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 213 |
| right | PKVRSTVAQHHEALVGHGFTHAHIVALSQH | |
| V0.1 HH | PAALGTVAVKYQDMIAALPEATHEAIVGVG | |
| pCLS33634 | KQWSGARALEALLTVAGELRGPPLQLDTGQ | |
| | LLKIAKRGGVTAVEAVHAWRNALTGAPLNL | |
| | TPEQVVAIASNIGGKQALETVQALLPVLCQ | |
| | AHGLTPQQVVAIASNNGGKQALETVQRLLP | |
| | VLCQAHGLTPEQVVAIASNIGGKQALETVQ | |
| | ALLPVLCQAHGLTPQQVVAIASNGGGKQAL | |
| | ETVQRLLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | QQVVAIASNNGGKQALETVQRLLPVLCQAH | |
| | GLTPQQVVAIASNNGGKQALETVQRLLPVL | |
| | CQAHGLTPQQVVAIASNGGGKQALETVQRL | |
| | LPVLCQAHGLTPQQVVAIASNNGGKQALET | |
| | VQRLLPVLCQAHGLTPEQVVAIASNIGGKQ | |
| | ALETVQALLPVLCQAHGLTPQQVVAIASNN | |
| | GGKQALETVQRLLPVLCQAHGLTPQQVVAI | |
| | ASNNGGKQALETVQRLLPVLCQAHGLTPEQ | |
| | VVAIASHDGGKQALETVQRLLPVLCQAHGL | |
| | TPQQVVAIASNGGGRPALESIVAQLSRPDP | |
| | ALAALTNDHLVALACLGGRPALDAVHHGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 214 |
| V0.2 RR | PKVRSTVAQHHEALVGHGFTHAHIVALSQH | |
| left | PAALGTVAVKYQDMIAALPEATHEAIVGVG | |
| pCLS33943 | KQWSGARALEALLTVAGELRGPPLQLDTGQ | |
| | LLKIAKRGGVTAVEAVHAWRNALTGAPLNL | |
| | TPQQVVAIASNGGGKQALETVQRLLPVLCQ | |
| | AHGLTPEQVVAIASHDGGKQALETVQRLLP | |
| | VLCQAHGLTPEQVVAIASHDGGKQALETVQ | |
| | RLLPVLCQAHGLTPEQVVAIASNIGGKQAL | |
| | ETVQALLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | EQVVAIASNIGGKQALETVQALLPVLCQAH | |
| | GLTPQQVVAIASNNGGKQALETVQRLLPVL | |
| | CQAHGLTPEQVVAIASHDGGKQALETVQRL | |
| | LPVLCQAHGLTPEQVVAIASNIGGKQALET | |
| | VQALLPVLCQAHGLTPEQVVAIASNIGGKQ | |
| | ALETVQALLPVLCQAHGLTPQQVVAIASNG | |
| | GGKQALETVQRLLPVLCQAHGLTPEQVVAI | |
| | ASNIGGKQALETVQALLPVLCQAHGLTPQQ | |
| | VVAIASNGGGKQALETVQRLLPVLCQAHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRINHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 215 |
| V0.2 | PKVRSTVAQHHEALVGHGFTHAHIVALSQH | |
| RR right | PAALGTVAVKYQDMIAALPEATHEAIVGVG | |
| pCLS33934 | KQWSGARALEALLTVAGELRGPPLQLDTGQ | |
| | LLKIAKRGGVTAVEAVHAWRNALTGAPLNL | |
| | TPEQVVAIASNIGGKQALETVQALLPVLCQ | |
| | AHGLTPQQVVAIASNNGGKQALETVQRLLP | |
| | VLCQAHGLTPEQVVAIASNIGGKQALETVQ | |
| | ALLPVLCQAHGLTPQQVVAIASNGGGKQAL | |
| | ETVQRLLPVLCQAHGLTPQQVVAIASNNGG | |
| | KQALETVQRLLPVLCQAHGLTPEQVVAIAS | |
| | NIGGKQALETVQALLPVLCQAHGLTPQQVV | |
| | AIASNNGGKQALETVQRLLPVLCQAHGLTP | |
| | QQVVAIASNNGGKQALETVQRLLPVLCQAH | |
| | GLTPQQVVAIASNNGGKQALETVQRLLPVL | |
| | CQAHGLTPQQVVAIASNGGGKQALETVQRL | |
| | LPVLCQAHGLTPQQVVAIASNNGGKQALET | |
| | VQRLLPVLCQAHGLTPEQVVAIASNIGGKQ | |
| | ALETVQALLPVLCQAHGLTPQQVVAIASNN | |
| | GGKQALETVQRLLPVLCQAHGLTPQQVVAI | |
| | ASNNGGKQALETVQRLLPVLCQAHGLTPEQ | |
| | VVAIASHDGGKQALETVQRLLPVLCQAHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 216 |
| V1 DD | PKVRSTVAQHHEALVGHGFTHAHIVALSQH | |
| left | PAALGTVAVKYQDMIAALPEATHEAIVGVG | |
| pCLS34716 | KQWSGARALEALLTVAGELRGPPLQLDTGQ | |
| | LLKIAKRGGVTAVEAVHAWRNALTGAPLNL | |
| | TPDQVVAIASNGGGKQALETVQRLLPVLCQ | |
| | DHGLTPDQVVAIASHDGGKQALETVQRLLP | |
| | VLCQDHGLTPDQVVAIASHDGGKQALETVQ | |
| | RLLPVLCQDHGLTPDQVVAIASNIGGKQAL | |
| | ETVQALLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNNGGKQALETVQRLLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASHDGGKQALETVQRL | |
| | LPVLCQDHGLTPDQVVAIASNIGGKQALET | |
| | VQALLPVLCQDHGLTPDQVVAIASNIGGKQ | |
| | ALETVQALLPVLCQDHGLTPDQVVAIASNG | |
| | GGKQALETVQRLLPVLCQDHGLTPDQVVAI | |
| | ASNIGGKQALETVQALLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 217 |
| V1 DD | PKVRSTVAQHHEALVGHGFTHAHIVALSQH | |
| right | PAALGTVAVKYQDMIAALPEATHEAIVGVG | |
| pCLS34717 | KQWSGARALEALLTVAGELRGPPLQLDTGQ | |
| | LLKIAKRGGVTAVEAVHAWRNALTGAPLNL | |
| | TPDQVVAIASNIGGKQALETVQALLPVLCQ | |
| | DHGLTPDQVVAIASNNGGKQALETVQRLLP | |
| | VLCQDHGLTPDQVVAIASNIGGKQALETVQ | |
| | ALLPVLCQDHGLTPDQVVAIASNGGGKQAL | |
| | ETVQRLLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNNGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNGGGKQALETVQRL | |
| | LPVLCQDHGLTPDQVVAIASNNGGKQALET | |
| | VQRLLPVLCQDHGLTPDQVVAIASNIGGKQ | |
| | ALETVQALLPVLCQDHGLTPDQVVAIASNN | |
| | GGKQALETVQRLLPVLCQDHGLTPDQVVAI | |
| | ASNNGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 218 |
| V1.2 | PKVRSTVAQHHEALVGHGFTHAHIVALSQH | |
| DD-RR left | PAALGTVAVKYQDMIAALPEATHEAIVGVG | |
| pCLS35196 | KQWSGARALEALLTVAGELRGPPLQLDTGQ | |
| | LLKIAKRGGVTAVEAVHAWRNALTGAPLNL | |
| | TPDQVVAIASNGGGKQALETVQRLLPVLCQ | |
| | DHGLTPDQVVAIASHDGGKQALETVQRLLP | |
| | VLCQDHGLTPDQVVAIASHDGGKQALETVQ | |
| | RLLPVLCQDHGLTPDQVVAIASNIGGKQAL | |
| | ETVQALLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNNGGKQALETVQRLLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASHDGGKQALETVQRL | |
| | LPVLCQDHGLTPDQVVAIASNIGGKQALET | |
| | VQALLPVLCQDHGLTPDQVVAIASNIGGKQ | |
| | ALETVQALLPVLCQDHGLTPDQVVAIASNG | |
| | GGKQALETVQRLLPVLCQDHGLTPDQVVAI | |
| | ASNIGGKQALETVQALLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CS1 TALEN | MGDPKKKRKVIDIADLRTLGYSQQQQEKIK | 219 |
| V1.2 | PKVRSTVAQHHEALVGHGFTHAHIVALSQH | |
| DD-RR right | PAALGTVAVKYQDMIAALPEATHEAIVGVG | |
| pCLS35197 | KQWSGARALEALLTVAGELRGPPLQLDTGQ | |
| | LLKIAKRGGVTAVEAVHAWRNALTGAPLNL | |
| | TPDQVVAIASNIGGKQALETVQALLPVLCQ | |
| | DHGLTPDQVVAIASNNGGKQALETVQRLLP | |
| | VLCQDHGLTPDQVVAIASNIGGKQALETVQ | |
| | ALLPVLCQDHGLTPDQVVAIASNGGGKQAL | |
| | ETVQRLLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNNGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNGGGKQALETVQRL | |
| | LPVLCQDHGLTPDQVVAIASNNGGKQALET | |
| | VQRLLPVLCQDHGLTPDQVVAIASNIGGKQ | |
| | ALETVQALLPVLCQDHGLTPDQVVAIASNN | |
| | GGKQALETVQRLLPVLCQDHGLTPDQVVAI | |
| | ASNNGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |

EXAMPLE 5

| | | |
|---|---|---|
| TALE-BE V2 | MGDPKKKRKVIDIADLRTLGYSQQQQEKIK | 220 |
| CD52-1 N | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNGGGKQALETVQRLLPVLCQ | |
| | DHGLTPDQVVAIASNGGGKQALETVQRLLP | |
| | VLCQDHGLTPDQVVAIASNGGGKQALETVQ | |
| | RLLPVLCQDHGLTPDQVVAIASNNGGKQAL | |
| | ETVQRLLPVLCQDHGLTPDQVVAIASNGGG | |
| | KQALETVQRLLPVLCQDHGLTPDQVVAIAS | |
| | HDGGKQALETVQRLLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNNGGKQALETVQRLLPVL | |
| | CQDHGLTPDQVVAIASNIGGKQALETVQAL | |
| | LPVLCQDHGLTPDQVVAIASNNGGKQALET | |
| | VQRLLPVLCQDHGLTPDQVVAIASNIGGKQ | |
| | ALETVQALLPVLCQDHGLTPDQVVAIASNN | |
| | GGKQALETVQRLLPVLCQDHGLTPDQVVAI | |
| | ASNGGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSGSYALGPYQISAPQLPAYNGQTVGTFYY | |
| | VNDAGGLESKVFSSGGPTPYPNYANAGHVE | |
| | GQSALFMRDNGISEGLVFHNNPEGTCGFCV | |
| | NMTETLLPENAKMTVVPPEGSGGSTNLSDI | |
| | IEKETGKQLVIQESILMLPEEVEEVIGNKP | |
| | ESDILVHTAYDESTDENVMLLTSDAPEYKP | |
| | WALVIQDSNGENKIKML | |
| TALE-BE V2 | MGDPKKKRKVIDIADLRTLGYSQQQQEKIK | 221 |
| CD52-1 C | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNIGGKQALETVQALLPVLCQ | |
| | DHGLTPDQVVAIASNGGGKQALETVQRLLP | |
| | VLCQDHGLTPDQVVAIASHDGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NIGGKQALETVQALLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASNGGGKQALET | |
| | VQRLLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSAIPVKRGATGETKVFTGNSNSPKSPTKG | |
| | GCSGGSTNLSDIIEKETGKQLVIQESILML | |
| | PEEVEEVIGNKPESDILVHTAYDESTDENV | |
| | MLLTSDAPEYKPWALVIQDSNGENKIKML | |
| TALE-BE V2 | MGDPKKKRKVIDIADLRTLGYSQQQQEKIK | 222 |
| CD52-2 N | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNGGGKQALETVQRLLPVLCQ | |
| | DHGLTPDQVVAIASNGGGKQALETVQRLLP | |
| | VLCQDHGLTPDQVVAIASNNGGKQALETVQ | |
| | RLLPVLCQDHGLTPDQVVAIASNGGGKQAL | |
| | ETVQRLLPVLCQDHGLTPDQVVAIASHDGG | |
| | KQALETVQRLLPVLCQDHGLTPDQVVAIAS | |
| | HDGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNIGGKQALETVQALLPVL | |
| | CQDHGLTPDQVVAIASNNGGKQALETVQRL | |
| | LPVLCQDHGLTPDQVVAIASNIGGKQALET | |
| | VQALLPVLCQDHGLTPDQVVAIASNNGGKQ | |
| | ALETVQRLLPVLCQDHGLTPDQVVAIASNG | |
| | GGKQALETVQRLLPVLCQDHGLTPDQVVAI | |
| | ASHDGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSGSYALGPYQISAPQLPAYNGQTVGTFYY | |
| | VNDAGGLESKVFSSGGPTPYPNYANAGHVE | |
| | GQSALFMRDNGISEGLVFHNNPEGTCGFCV | |
| | NMTETLLPENAKMTVVPPEGSGGSTNLSDI | |
| | IEKETGKQLVIQESILMLPEEVEEVIGNKP | |
| | ESDILVHTAYDESTDENVMLLTSDAPEYKP | |
| | WALVIQDSNGENKIKML | |
| TALE-BE V2 | MGDPKKKRKVIDIADLRTLGYSQQQQEKIK | 223 |
| CD52-2 C | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNIGGKQALETVQALLPVLCQ | |
| | DHGLTPDQVVAIASNGGGKQALETVQRLLP | |
| | VLCQDHGLTPDQVVAIASHDGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NIGGKQALETVQALLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASNGGGKQALET | |
| | VQRLLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSAIPVKRGATGETKVFTGNSNSPKSPTKG | |
| | GCSGGSTNLSDIIEKETGKQLVIQESILML | |
| | PEEVEEVIGNKPESDILVHTAYDESTDENV | |
| | MLLTSDAPEYKPWALVIQDSNGENKIKML | |
| TALE-BE V2 | MGDPKKKRKVIDIADLRTLGYSQQQQEKIK | 224 |
| CD52-3 N | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNGGGKQALETVQRLLPVLCQ | |
| | DHGLTPDQVVAIASNNGGKQALETVQRLLP | |
| | VLCQDHGLTPDQVVAIASNGGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NGGGKQALETVQRLLPVLCQDHGLTPDQVV | |
| | AIASNNGGKQALETVQRLLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASNIGGKQALETVQAL | |
| | LPVLCQDHGLTPDQVVAIASNNGGKQALET | |
| | VQRLLPVLCQDHGLTPDQVVAIASNGGGKQ | |
| | ALETVQRLLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | GSGSYALGPYQISAPQLPAYNGQTVGTFYY | |
| | VNDAGGLESKVFSSGGPTPYPNYANAGHVE | |
| | GQSALFMRDNGISEGLVFHNNPEGTCGFCV | |
| | NMTETLLPENAKMTVVPPEGSGGSTNLSDI | |
| | IEKETGKQLVIQESILMLPEEVEEVIGNKP | |
| | ESDILVHTAYDESTDENVMLLTSDAPEYKP | |
| | WALVIQDSNGENKIKML | |
| TALE-BE V2 | MGDPKKKRKVIDIADLRTLGYSQQQQEKIK | 225 |
| CD52-3 C | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNIGGKQALETVQALLPVLCQ | |
| | DHGLTPDQVVAIASNGGGKQALETVQRLLP | |
| | VLCQDHGLTPDQVVAIASHDGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NIGGKQALETVQALLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASNGGGKQALET | |
| | VQRLLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSAIPVKRGATGETKVFTGNSNSPKSPTKG | |
| | GCSGGSTNLSDIIEKETGKQLVIQESILML | |
| | PEEVEEVIGNKPESDILVHTAYDESTDENV | |
| | MLLTSDAPEYKPWALVIQDSNGENKIKML | |
| TALE-BE V2 | MGDPKKKRKVIDIADLRTLGYSQQQQEKIK | 226 |
| CD52-4 N | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASNNGGKQALETVQRLLPVLCQ | |
| | DHGLTPDQVVAIASNNGGKQALETVQRLLP | |
| | VLCQDHGLTPDQVVAIASHDGGKQALETVQ | |
| | RLLPVLCQDHGLTPDQVVAIASNGGGKQAL | |
| | ETVQRLLPVLCQDHGLTPDQVVAIASNNGG | |
| | KQALETVQRLLPVLCQDHGLTPDQVVAIAS | |
| | NNGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASNNGGKQALET | |
| | VQRLLPVLCQDHGLTPDQVVAIASNGGGKQ | |
| | ALETVQRLLPVLCQDHGLTPDQVVAIASNG | |
| | GGKQALETVQRLLPVLCQDHGLTPDQVVAI | |
| | ASNGGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | GSGSYALGPYQISAPQLPAYNGQTVGTFYY | |
| | VNDAGGLESKVFSSGGPTPYPNYANAGHVE | |
| | GQSALFMRDNGISEGLVFHNNPEGTCGFCV | |
| | NMTETLLPENAKMTVVPPEGSGGSTNLSDI | |
| | IEKETGKQLVIQESILMLPEEVEEVIGNKP | |
| | ESDILVHTAYDESTDENVMLLTSDAPEYKP | |
| | WALVIQDSNGENKIKML | |
| TALE-BE V2 | MGDPKKKRKVIDIADLRTLGYSQQQQEKIK | 227 |
| CD52-4 C | PKVRSTVAQHHEALVGHGFTHAHIVALSQH | |
| | PAALGTVAVKYQDMIAALPEATHEAIVGVG | |
| | KQWSGARALEALLTVAGELRGPPLQLDTGQ | |
| | LLKIAKRGGVTAVEAVHAWRNALTGAPLNL | |
| | TPDQVVAIASHDGGKQALETVQRLLPVLCQ | |
| | DHGLTPDQVVAIASHDGGKQALETVQRLLP | |
| | VLCQDHGLTPDQVVAIASNGGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NGGGKQALETVQRLLPVLCQDHGLTPDQVV | |
| | AIASNIGGKQALETVQALLPVLCQDHGLTP | |
| | DQVVAIASHDGGKQALETVQRLLPVLCQDH | |
| | GLTPDQVVAIASNIGGKQALETVQALLPVL | |
| | CQDHGLTPDQVVAIASNNGGKQALETVQRL | |
| | LPVLCQDHGLTPDQVVAIASNIGGKQALET | |
| | VQALLPVLCQDHGLTPDQVVAIASNGGGKQ | |
| | ALETVQRLLPVLCQDHGLTPDQVVAIASNI | |
| | GGKQALETVQALLPVLCQDHGLTPDQVVAI | |
| | ASHDGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | GSAIPVKRGATGETKVFTGNSNSPKSPTKG | |
| | GCSGGSTNLSDIIEKETGKQLVIQESILML | |
| | PEEVEEVIGNKPESDILVHTAYDESTDENV | |
| | MLLTSDAPEYKPWALVIQDSNGENKIKML | |
| TGFBRII | MGDPKKKRKVIDIADLRTLGYSQQQQEKIK | 267 |
| TALEN- | PKVRSTVAQHHEALVGHGFTHAHIVALSQH | |
| classical | PAALGTVAVKYQDMIAALPEATHEAIVGVG | |
| (V0) | KQWSGARALEALLTVAGELRGPPLQLDTGQ | |
| left | LLKIAKRGGVTAVEAVHAWRNALTGAPLNL | |
| pCLS32967 | TPEQVVAIASHDGGKQALETVQRLLPVLCQ | |
| | AHGLTPEQVVAIASHDGGKQALETVQRLLP | |
| | VLCQAHGLTPEQVVAIASHDGGKQALETVQ | |
| | RLLPVLCQAHGLTPQQVVAIASNGGGKQAL | |
| | ETVQRLLPVLCQAHGLTPEQVVAIASNIGG | |
| | KQALETVQALLPVLCQAHGLTPQQVVAIAS | |
| | NGGGKQALETVQRLLPVLCQAHGLTPQQVV | |
| | AIASNNGGKQALETVQRLLPVLCQAHGLTP | |
| | EQVVAIASNIGGKQALETVQALLPVLCQAH | |
| | GLTPQQVVAIASNNGGKQALETVQRLLPVL | |
| | CQAHGLTPQQVVAIASNNGGKQALETVQRL | |
| | LPVLCQAHGLTPEQVVAIASNIGGKQALET | |
| | VQALLPVLCQAHGLTPQQVVAIASNNGGKQ | |
| | ALETVQRLLPVLCQAHGLTPQQVVAIASNG | |
| | GGKQALETVQRLLPVLCQAHGLTPEQVVAI | |
| | ASNIGGKQALETVQALLPVLCQAHGLTPQQ | |
| | VVAIASNGGGKQALETVQRLLPVLCQAHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| TGFBRII | MGDPKKKRKVIDIADLRTLGYSQQQQEKIK | 268 |
| TALEN- | PKVRSTVAQHHEALVGHGFTHAHIVALSQH | |
| classical | PAALGTVAVKYQDMIAALPEATHEAIVGVG | |
| (V0) | KQWSGARALEALLTVAGELRGPPLQLDTGQ | |
| right | LLKIAKRGGVTAVEAVHAWRNALTGAPLNL | |
| pCLS32968 | TPQQVVAIASNNGGKQALETVQRLLPVLCQ | |
| | AHGLTPEQVVAIASNIGGKQALETVQALLP | |
| | VLCQAHGLTPQQVVAIASNNGGKQALETVQ | |
| | RLLPVLCQAHGLTPEQVVAIASNIGGKQAL | |
| | ETVQALLPVLCQAHGLTPEQVVAIASNIGG | |
| | KQALETVQALLPVLCQAHGLTPQQVVAIAS | |
| | NNGGKQALETVQRLLPVLCQAHGLTPEQVV | |
| | AIASNIGGKQALETVQALLPVLCQAHGLTP | |
| | QQVVAIASNGGGKQALETVQRLLPVLCQAH | |
| | GLTPQQVVAIASNNGGKQALETVQRLLPVL | |
| | CQAHGLTPQQVVAIASNGGGKQALETVQRL | |
| | LPVLCQAHGLTPEQVVAIASHDGGKQALET | |
| | VQRLLPVLCQAHGLTPEQVVAIASHDGGKQ | |
| | ALETVQRLLPVLCQAHGLTPQQVVAIASNG | |
| | GGKQALETVQRLLPVLCQAHGLTPQQVVAI | |
| | ASNGGGKQALETVQRLLPVLCQAHGLTPEQ | |
| | VVAIASHDGGKQALETVQRLLPVLCQAHGL | |
| | TPQQVVAIASNGGGRPALESIVAQLSRPDP | |
| | ALAALTNDHLVALACLGGRPALDAVKKGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |

EXAMPLE 7

| | | |
|---|---|---|
| TIGIT left | MGDPKKKRKVIDIADLRTLGYSQQQQEKIK | 269 |
| V2 | PKVRSTVAQHHEALVGHGFTHAHIVALSQH | |
| TALEN | PAALGTVAVKYQDMIAALPEATHEAIVGVG | |
| pCLS39233 | KQWSGARALEALLTVAGELRGPPLQLDTGQ | |
| | LLKIAKRGGVTAVEAVHAWRNALTGAPLNL | |
| | TPDQVVAIASNNGGKQALETVQRLLPVLCQ | |
| | DHGLTPDQVVAIASNGGGKQALETVQRLLP | |
| | VLCQDHGLTPDQVVAIASHDGGKQALETVQ | |
| | RLLPVLCQDHGLTPDQVVAIASNIGGKQAL | |
| | ETVQALLPVLCQDHGLTPDQVVAIASHDGG | |
| | KQALETVQRLLPVLCQDHGLTPDQVVAIAS | |
| | HDGGKQALETVQRLLPVLCQDHGLTPDQVV | |
| | AIASNGGGKQALETVQRLLPVLCQDHGLTP | |
| | DQVVAIASHDGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASHDGGKQALET | |
| | VQRLLPVLCQDHGLTPDQVVAIASNGGGKQ | |
| | ALETVQRLLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| TIGIT right | MGDPKKKRKVIDIADLRTLGYSQQQQEKIK | 270 |
| V2 | PKVRSTVAQHHEALVGHGFTHAHIVALSQH | |
| TALEN | PAALGTVAVKYQDMIAALPEATHEAIVGVG | |
| pCLS39234 | KQWSGARALEALLTVAGELRGPPLQLDTGQ | |
| | LLKIAKRGGVTAVEAVHAWRNALTGAPLNL | |
| | TPDQVVAIASHDGGKQALETVQRLLPVLCQ | |
| | DHGLTPDQVVAIASHDGGKQALETVQRLLP | |
| | VLCQDHGLTPDQVVAIASHDGGKQALETVQ | |
| | RLLPVLCQDHGLTPDQVVAIASNIGGKQAL | |
| | ETVQALLPVLCQDHGLTPDQVVAIASNNGG | |
| | KQALETVQRLLPVLCQDHGLTPDQVVAIAS | |
| | NGGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNIGGKQALETVQALLPVL | |
| | CQDHGLTPDQVVAIASHDGGKQALETVQRL | |
| | LPVLCQDHGLTPDQVVAIASHDGGKQALET | |
| | VQRLLPVLCQDHGLTPDQVVAIASNGGGKQ | |
| | ALETVQRLLPVLCQDHGLTPDQVVAIASNN | |
| | GGKQALETVQRLLPVLCQDHGLTPDQVVAI | |
| | ASNNGGKQALETVQRLLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (1) | MGDPKKKRKVIDIADLRTLGYSQQQQEKIK | 271 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS38090 | LLKIAKRGGVTAVEAVHAWRNALTGAPLNL | |
| | TPDQVVAIASHDGGKQALETVQRLLPVLCQ | |
| | DHGLTPDQVVAIASNNGGKQALETVQRLLP | |
| | VLCQDHGLTPDQVVAIASNIGGKQALETVQ | |
| | ALLPVLCQDHGLTPDQVVAIASNNGGKQAL | |
| | ETVQRLLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNNGGKQALETVQRLLPVLCQDHGLTP | |
| | DQVVAIASNNGGKQALETVQRLLPVLCQDH | |
| | GLTPDQVVAIASNGGGKQALETVQRLLPVL | |
| | CQDHGLTPDQVVAIASNNGGKQALETVQRL | |
| | LPVLCQDHGLTPDQVVAIASNNGGKQALET | |
| | VQRLLPVLCQDHGLTPDQVVAIASHDGGKQ | |
| | ALETVQRLLPVLCQDHGLTPDQVVAIASNI | |
| | GGKQALETVQALLPVLCQDHGLTPDQVVAI | |
| | ASNNGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (1) | MGDPKKKRKVIDIADLRTLGYSQQQQEKIK | 272 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS38091 | LLKIAKRGGVTAVEAVHAWRNALTGAPLNL | |
| | TPDQVVAIASNGGGKQALETVQRLLPVLCQ | |
| | DHGLTPDQVVAIASNGGGKQALETVQRLLP | |
| | VLCQDHGLTPDQVVAIASNNGGKQALETVQ | |
| | RLLPVLCQDHGLTPDQVVAIASNNGGKQAL | |
| | ETVQRLLPVLCQDHGLTPDQVVAIASHDGG | |
| | KQALETVQRLLPVLCQDHGLTPDQVVAIAS | |
| | NGGGKQALETVQRLLPVLCQDHGLTPDQVV | |
| | AIASHDGGKQALETVQRLLPVLCQDHGLTP | |
| | DQVVAIASNIGGKQALETVQALLPVLCQDH | |
| | GLTPDQVVAIASHDGGKQALETVQRLLPVL | |
| | CQDHGLTPDQVVAIASNGGGKQALETVQRL | |
| | LPVLCQDHGLTPDQVVAIASHDGGKQALET | |
| | VQRLLPVLCQDHGLTPDQVVAIASNGGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (2) | MGDPKKKRKVIDIADLRTLGYSQQQQEKIK | 273 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS38094 | LLKIAKRGGVTAVEAVHAWRNALTGAPLNL | |
| | TPDQVVAIASNNGGKQALETVQRLLPVLCQ | |
| | DHGLTPDQVVAIASNNGGKQALETVQRLLP | |
| | VLCQDHGLTPDQVVAIASNGGGKQALETVQ | |
| | RLLPVLCQDHGLTPDQVVAIASNIGGKQAL | |
| | ETVQALLPVLCQDHGLTPDQVVAIASNGGG | |
| | KQALETVQRLLPVLCQDHGLTPDQVVAIAS | |
| | NGGGKQALETVQRLLPVLCQDHGLTPDQVV | |
| | AIASNNGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNNGGKQALETVQRL | |
| | LPVLCQDHGLTPDQVVAIASNGGGKQALET | |
| | VQRLLPVLCQDHGLTPDQVVAIASNGGGKQ | |
| | ALETVQRLLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (2) | MGDPKKKRKVIDIADLRTLGYSQQQQEKIK | 274 |
| right | PKVRSTVAQHHEALVGHGFTHAHIVALSQH | |
| TALEN | PAALGTVAVKYQDMIAALPEATHEAIVGVG | |
| pCLS38095 | KQWSGARALEALLTVAGELRGPPLQLDTGQ | |
| | LLKIAKRGGVTAVEAVHAWRNALTGAPLNL | |
| | TPDQVVAIASHDGGKQALETVQRLLPVLCQ | |
| | DHGLTPDQVVAIASNGGGKQALETVQRLLP | |
| | VLCQDHGLTPDQVVAIASNNGGKQALETVQ | |
| | RLLPVLCQDHGLTPDQVVAIASHDGGKQAL | |
| | ETVQRLLPVLCQDHGLTPDQVVAIASNIGG | |
| | KQALETVQALLPVLCQDHGLTPDQVVAIAS | |
| | NNGGKQALETVQRLLPVLCQDHGLTPDQVV | |
| | AIASNNGGKQALETVQRLLPVLCQDHGLTP | |
| | DQVVAIASNGGGKQALETVQRLLPVLCQDH | |
| | GLTPDQVVAIASNNGGKQALETVQRLLPVL | |
| | CQDHGLTPDQVVAIASNGGGKQALETVQRL | |
| | LPVLCQDHGLTPDQVVAIASNGGGKQALET | |
| | VQRLLPVLCQDHGLTPDQVVAIASNNGGKQ | |
| | ALETVQRLLPVLCQDHGLTPDQVVAIASNG | |
| | GGKQALETVQRLLPVLCQDHGLTPDQVVAI | |
| | ASHDGGKQALETVQRLLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (3) | MGDPKKKRKVIDIADLRTLGYSQQQQEKIK | 275 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS38086 | LLKIAKRGGVTAVEAVHAWRNALTGAPLNL | |
| | TPDQVVAIASNNGGKQALETVQRLLPVLCQ | |
| | DHGLTPDQVVAIASHDGGKQALETVQRLLP | |
| | VLCQDHGLTPDQVVAIASHDGGKQALETVQ | |
| | RLLPVLCQDHGLTPDQVVAIASNGGGKQAL | |
| | ETVQRLLPVLCQDHGLTPDQVVAIASNNGG | |
| | KQALETVQRLLPVLCQDHGLTPDQVVAIAS | |
| | HDGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNNGGKQALETVQRL | |
| | LPVLCQDHGLTPDQVVAIASNNGGKQALET | |
| | VQRLLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CISH (3) | MGDPKKKRKVIDIADLRTLGYSQQQQEKIK | 276 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS38087 | LLKIAKRGGVTAVEAVHAWRNALTGAPLNL | |
| | TPDQVVAIASNNGGKQALETVQRLLPVLCQ | |
| | DHGLTPDQVVAIASNNGGKQALETVQRLLP | |
| | VLCQDHGLTPDQVVAIASNNGGKQALETVQ | |
| | RLLPVLCQDHGLTPDQVVAIASHDGGKQAL | |
| | ETVQRLLPVLCQDHGLTPDQVVAIASNGGG | |
| | KQALETVQRLLPVLCQDHGLTPDQVVAIAS | |
| | NNGGKQALETVQRLLPVLCQDHGLTPDQVV | |
| | AIASNNGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNGGGKQALETVQRL | |
| | LPVLCQDHGLTPDQVVAIASNIGGKQALET | |
| | VQALLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CD38 (1) | MGDPKKKRKVIDIADLRTLGYSQQQQEKIK | 277 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS37948 | LLKIAKRGGVTAVEAVHAWRNALTGAPLNL | |
| | TPDQVVAIASNGGGKQALETVQRLLPVLCQ | |
| | DHGLTPDQVVAIASNGGGKQALETVQRLLP | |
| | VLCQDHGLTPDQVVAIASHDGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | NNGGKQALETVQRLLPVLCQDHGLTPDQVV | |
| | AIASNIGGKQALETVQALLPVLCQDHGLTP | |
| | DQVVAIASNNGGKQALETVQRLLPVLCQDH | |
| | GLTPDQVVAIASNIGGKQALETVQALLPVL | |
| | CQDHGLTPDQVVAIASHDGGKQALETVQRL | |
| | LPVLCQDHGLTPDQVVAIASHDGGKQALET | |
| | VQRLLPVLCQDHGLTPDQVVAIASNNGGKQ | |
| | ALETVQRLLPVLCQDHGLTPDQVVAIASNG | |
| | GGKQALETVQRLLPVLCQDHGLTPDQVVAI | |
| | ASHDGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CD38 (1) | MGDPKKKRKVIDIADLRTLGYSQQQQEKIK | 278 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS37949 | LLKIAKRGGVTAVEAVHAWRNALTGAPLNL | |
| | TPDQVVAIASNNGGKQALETVQRLLPVLCQ | |
| | DHGLTPDQVVAIASNIGGKQALETVQALLP | |
| | VLCQDHGLTPDQVVAIASNIGGKQALETVQ | |
| | ALLPVLCQDHGLTPDQVVAIASNGGGKQAL | |
| | ETVQRLLPVLCQDHGLTPDQVVAIASNGGG | |
| | KQALETVQRLLPVLCQDHGLTPDQVVAIAS | |
| | NGGGKQALETVQRLLPVLCQDHGLTPDQVV | |
| | AIASHDGGKQALETVQRLLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASNGGGKQALETVQRL | |
| | LPVLCQDHGLTPDQVVAIASNNGGKQALET | |
| | VQRLLPVLCQDHGLTPDQVVAIASNGGGKQ | |
| | ALETVQRLLPVLCQDHGLTPDQVVAIASNI | |
| | GGKQALETVQALLPVLCQDHGLTPDQVVAI | |
| | ASHDGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CD38 (2) | MGDPKKKRKVIDIADLRTLGYSQQQQEKIK | 279 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS37931 | LLKIAKRGGVTAVEAVHAWRNALTGAPLNL | |
| | TPDQVVAIASNNGGKQALETVQRLLPVLCQ | |
| | DHGLTPDQVVAIASNNGGKQALETVQRLLP | |
| | VLCQDHGLTPDQVVAIASNIGGKQALETVQ | |
| | ALLPVLCQDHGLTPDQVVAIASNNGGKQAL | |
| | ETVQRLLPVLCQDHGLTPDQVVAIASHDGG | |
| | KQALETVQRLLPVLCQDHGLTPDQVVAIAS | |
| | HDGGKQALETVQRLLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNIGGKQALETVQALLPVL | |
| | CQDHGLTPDQVVAIASNGGGKQALETVQRL | |
| | LPVLCQDHGLTPDQVVAIASNNGGKQALET | |
| | VQRLLPVLCQDHGLTPDQVVAIASNNGGKQ | |
| | ALETVQRLLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| CD38 (2) | MGDPKKKRKVIDIADLRTLGYSQQQQEKIK | 280 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS37932 | LLKIAKRGGVTAVEAVHAWRNALTGAPLNL | |
| | TPDQVVAIASNGGGKQALETVQRLLPVLCQ | |
| | DHGLTPDQVVAIASNNGGKQALETVQRLLP | |
| | VLCQDHGLTPDQVVAIASNGGGKQALETVQ | |
| | RLLPVLCQDHGLTPDQVVAIASHDGGKQAL | |
| | ETVQRLLPVLCQDHGLTPDQVVAIASHDGG | |
| | KQALETVQRLLPVLCQDHGLTPDQVVAIAS | |
| | HDGGKQALETVQRLLPVLCQDHGLTPDQVV | |
| | AIASHDGGKQALETVQRLLPVLCQDHGLTP | |
| | DQVVAIASNNGGKQALETVQRLLPVLCQDH | |
| | GLTPDQVVAIASNNGGKQALETVQRLLPVL | |
| | CQDHGLTPDQVVAIASNIGGKQALETVQAL | |
| | LPVLCQDHGLTPDQVVAIASHDGGKQALET | |
| | VQRLLPVLCQDHGLTPDQVVAIASNIGGKQ | |
| | ALETVQALLPVLCQDHGLTPDQVVAIASHD | |
| | GGKQALETVQRLLPVLCQDHGLTPDQVVAI | |
| | ASHDGGKQALETVQRLLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| IgH (1) | MGDPKKKRKVIDIADLRTLGYSQQQQEKIK | 281 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39197 | LLKIAKRGGVTAVEAVHAWRNALTGAPLNL | |
| | TPDQVVAIASNNGGKQALETVQRLLPVLCQ | |
| | DHGLTPDQVVAIASNIGGKQALETVQALLP | |
| | VLCQDHGLTPDQVVAIASNGGGKQALETVQ | |
| | RLLPVLCQDHGLTPDQVVAIASNIGGKQAL | |
| | ETVQALLPVLCQDHGLTPDQVVAIASNGGG | |
| | KQALETVQRLLPVLCQDHGLTPDQVVAIAS | |
| | NNGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNGGGKQALETVQRLLPVL | |
| | CQDHGLTPDQVVAIASHDGGKQALETVQRL | |
| | LPVLCQDHGLTPDQVVAIASNGGGKQALET | |
| | VQRLLPVLCQDHGLTPDQVVAIASNNGGKQ | |
| | ALETVQRLLPVLCQDHGLTPDQVVAIASNN | |
| | GGKQALETVQRLLPVLCQDHGLTPDQVVAI | |
| | ASNIGGKQALETVQALLPVLCQDHGLTPDQ | |
| | VVAIASNIGGKQALETVQALLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| IgH (1) | MGDPKKKRKVIDIADLRTLGYSQQQQEKIK | 282 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39198 | LLKIAKRGGVTAVEAVHAWRNALTGAPLNL | |
| | TPDQVVAIASNIGGKQALETVQALLPVLCQ | |
| | DHGLTPDQVVAIASNGGGKQALETVQRLLP | |
| | VLCQDHGLTPDQVVAIASNGGGKQALETVQ | |
| | RLLPVLCQDHGLTPDQVVAIASNGGGKQAL | |
| | ETVQRLLPVLCQDHGLTPDQVVAIASHDGG | |
| | KQALETVQRLLPVLCQDHGLTPDQVVAIAS | |
| | NGGGKQALETVQRLLPVLCQDHGLTPDQVV | |
| | AIASNGGGKQALETVQRLLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASHDGGKQALETVQRL | |
| | LPVLCQDHGLTPDQVVAIASNGGGKQALET | |
| | VQRLLPVLCQDHGLTPDQVVAIASNNGGKQ | |
| | ALETVQRLLPVLCQDHGLTPDQVVAIASNI | |
| | GGKQALETVQALLPVLCQDHGLTPDQVVAI | |
| | ASNNGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| IgH (2) | MGDPKKKRKVIDIADLRTLGYSQQQQEKIK | 283 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39194 | LLKIAKRGGVTAVEAVHAWRNALTGAPLNL | |
| | TPDQVVAIASNNGGKQALETVQRLLPVLCQ | |
| | DHGLTPDQVVAIASNIGGKQALETVQALLP | |
| | VLCQDHGLTPDQVVAIASNNGGKQALETVQ | |
| | RLLPVLCQDHGLTPDQVVAIASNGGGKQAL | |
| | ETVQRLLPVLCQDHGLTPDQVVAIASNNGG | |
| | KQALETVQRLLPVLCQDHGLTPDQVVAIAS | |
| | NIGGKQALETVQALLPVLCQDHGLTPDQVV | |
| | AIASNGGGKQALETVQRLLPVLCQDHGLTP | |
| | DQVVAIASNIGGKQALETVQALLPVLCQDH | |
| | GLTPDQVVAIASNGGGKQALETVQRLLPVL | |
| | CQDHGLTPDQVVAIASNNGGKQALETVQRL | |
| | LPVLCQDHGLTPDQVVAIASNGGGKQALET | |
| | VQRLLPVLCQDHGLTPDQVVAIASNNGGKQ | |
| | ALETVQRLLPVLCQDHGLTPDQVVAIASNG | |
| | GGKQALETVQRLLPVLCQDHGLTPDQVVAI | |
| | ASHDGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| IgH (2) | MGDPKKKRKVIDIADLRTLGYSQQQQEKIK | 284 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39195 | LLKIAKRGGVTAVEAVHAWRNALTGAPLNL | |
| | TPDQVVAIASHDGGKQALETVQRLLPVLCQ | |
| | DHGLTPDQVVAIASNGGGKQALETVQRLLP | |
| | VLCQDHGLTPDQVVAIASNGGGKQALETVQ | |
| | RLLPVLCQDHGLTPDQVVAIASNIGGKQAL | |
| | ETVQALLPVLCQDHGLTPDQVVAIASNNGG | |
| | KQALETVQRLLPVLCQDHGLTPDQVVAIAS | |
| | HDGGKQALETVQRLLPVLCQDHGLTPDQVV | |
| | AIASNGGGKQALETVQRLLPVLCQDHGLTP | |
| | DQVVAIASNNGGKQALETVQRLLPVLCQDH | |
| | GLTPDQVVAIASNIGGKQALETVQALLPVL | |
| | CQDHGLTPDQVVAIASNNGGKQALETVQRL | |
| | LPVLCQDHGLTPDQVVAIASHDGGKQALET | |
| | VQRLLPVLCQDHGLTPDQVVAIASNGGGKQ | |
| | ALETVQRLLPVLCQDHGLTPDQVVAIASNG | |
| | GGKQALETVQRLLPVLCQDHGLTPDQVVAI | |
| | ASNNGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| GADPH (1) | MGDPKKKRKVIDIADLRTLGYSQQQQEKIK | 285 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39327 | LLKIAKRGGVTAVEAVHAWRNALTGAPLNL | |
| | TPDQVVAIASHDGGKQALETVQRLLPVLCQ | |
| | DHGLTPDQVVAIASNGGGKQALETVQRLLP | |
| | VLCQDHGLTPDQVVAIASNGGGKQALETVQ | |
| | RLLPVLCQDHGLTPDQVVAIASNIGGKQAL | |
| | ETVQALLPVLCQDHGLTPDQVVAIASNGGG | |
| | KQALETVQRLLPVLCQDHGLTPDQVVAIAS | |
| | NGGGKQALETVQRLLPVLCQDHGLTPDQVV | |
| | AIASHDGGKQALETVQRLLPVLCQDHGLTP | |
| | DQVVAIASNGGGKQALETVQRLLPVLCQDH | |
| | GLTPDQVVAIASNIGGKQALETVQALLPVL | |
| | CQDHGLTPDQVVAIASNNGGKQALETVQRL | |
| | LPVLCQDHGLTPDQVVAIASNNGGKQALET | |
| | VQRLLPVLCQDHGLTPDQVVAIASNNGGKQ | |
| | ALETVQRLLPVLCQDHGLTPDQVVAIASNG | |
| | GGKQALETVQRLLPVLCQDHGLTPDQVVAI | |
| | ASHDGGKQALETVQRLLPVLCQDHGLTPDQ | |
| | VVAIASNGGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| GADPH (1) | MGDPKKKRKVIDIADLRTLGYSQQQQEKIK | 286 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39329 | LLKIAKRGGVTAVEAVHAWRNALTGAPLNL | |
| | TPDQVVAIASNGGGKQALETVQRLLPVLCQ | |
| | DHGLTPDQVVAIASNNGGKQALETVQRLLP | |
| | VLCQDHGLTPDQVVAIASNIGGKQALETVQ | |
| | ALLPVLCQDHGLTPDQVVAIASHDGGKQAL | |
| | ETVQRLLPVLCQDHGLTPDQVVAIASNIGG | |
| | KQALETVQALLPVLCQDHGLTPDQVVAIAS | |
| | HDGGKQALETVQRLLPVLCQDHGLTPDQVV | |
| | AIASNIGGKQALETVQALLPVLCQDHGLTP | |
| | DQVVAIASNIGGKQALETVQALLPVLCQDH | |
| | GLTPDQVVAIASNNGGKQALETVQRLLPVL | |
| | CQDHGLTPDQVVAIASHDGGKQALETVQRL | |
| | LPVLCQDHGLTPDQVVAIASHDGGKQALET | |
| | VQRLLPVLCQDHGLTPDQVVAIASHDGGKQ | |
| | ALETVQRLLPVLCQDHGLTPDQVVAIASNI | |
| | GGKQALETVQALLPVLCQDHGLTPDQVVAI | |
| | ASNNGGKQALETVQRLLPVLCQDHGLTPDQ | |
| | VVAIASHDGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| GADPH (2) | MGDPKKKRKVIDIADLRTLGYSQQQQEKIK | 287 |
| left | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39357 | LLKIAKRGGVTAVEAVHAWRNALTGAPLNL | |
| | TPDQVVAIASNGGGKQALETVQRLLPVLCQ | |
| | DHGLTPDQVVAIASNGGGKQALETVQRLLP | |
| | VLCQDHGLTPDQVVAIASNNGGKQALETVQ | |
| | RLLPVLCQDHGLTPDQVVAIASHDGGKQAL | |
| | ETVQRLLPVLCQDHGLTPDQVVAIASNGGG | |
| | KQALETVQRLLPVLCQDHGLTPDQVVAIAS | |
| | NGGGKQALETVQRLLPVLCQDHGLTPDQVV | |
| | AIASHDGGKQALETVQRLLPVLCQDHGLTP | |
| | DQVVAIASHDGGKQALETVQRLLPVLCQDH | |
| | GLTPDQVVAIASHDGGKQALETVQRLLPVL | |
| | CQDHGLTPDQVVAIASNNGGKQALETVQRL | |
| | LPVLCQDHGLTPDQVVAIASHDGGKQALET | |
| | VQRLLPVLCQDHGLTPDQVVAIASNGGGKQ | |
| | ALETVQRLLPVLCQDHGLTPDQVVAIASHD | |
| | GGKQALETVQRLLPVLCQDHGLTPDQVVAI | |
| | ASNIGGKQALETVQALLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |
| GADPH (2) | MGDPKKKRKVIDIADLRTLGYSQQQQEKIK | 288 |
| right | PKVRSTVAQHHEALVGHGFTHAHIVALSQH | |
| TALEN | PAALGTVAVKYQDMIAALPEATHEAIVGVG | |
| V2 | KQWSGARALEALLTVAGELRGPPLQLDTGQ | |
| pCLS39358 | LLKIAKRGGVTAVEAVHAWRNALTGAPLNL | |
| | TPDQVVAIASNNGGKQALETVQRLLPVLCQ | |
| | DHGLTPDQVVAIASNNGGKQALETVQRLLP | |
| | VLCQDHGLTPDQVVAIASNGGGKQALETVQ | |
| | RLLPVLCQDHGLTPDQVVAIASNNGGKQAL | |
| | ETVQRLLPVLCQDHGLTPDQVVAIASHDGG | |
| | KQALETVQRLLPVLCQDHGLTPDQVVAIAS | |
| | HDGGKQALETVQRLLPVLCQDHGLTPDQVV | |
| | AIASNIGGKQALETVQALLPVLCQDHGLTP | |
| | DQVVAIASNNGGKQALETVQRLLPVLCQDH | |
| | GLTPDQVVAIASHDGGKQALETVQRLLPVL | |
| | CQDHGLTPDQVVAIASNGGGKQALETVQRL | |
| | LPVLCQDHGLTPDQVVAIASNGGGKQALET | |
| | VQRLLPVLCQDHGLTPDQVVAIASHDGGKQ | |
| | ALETVQRLLPVLCQDHGLTPDQVVAIASHD | |
| | GGKQALETVQRLLPVLCQDHGLTPDQVVAI | |
| | ASNGGGKQALETVQRLLPVLCQDHGLTPDQ | |
| | VVAIASNNGGKQALETVQRLLPVLCQDHGL | |
| | TPQQVVAIASNGGGRPALESIVAQLSRPDP | |
| | ALAALTNDHLVALACLGGRPALDAVRRGLG | |
| | DPISRSQLVKSELEEKKSELRHKLKYVPHE | |
| | YIELIEIARNSTQDRILEMKVMEFFMKVYG | |
| | YRGKHLGGSRKPDGAIYTVGSPIDYGVIVD | |
| | TKAYSGGYNLPIGQADEMQRYVEENQTRNK | |
| | HINPNEWWKVYPSSVTEFKFLFVSGHFKGN | |
| | YKAQLTRLNHITNCNGAVLSVEELLIGGEM | |
| | IKAGTLTLEEVRRKFNNGEINFAAD | |

TABLE 6
Polynucleotide sequences used
in the Examples

| | | |
|---|---|---|
| | | SEQ |
| | | ID |
| | Polynucleotide | NO: |
| Sequence name | sequences | # |

EXAMPLE 2/3/4 (target sequences)

| | | |
|---|---|---|
| Genomic CS1 | TTCCAGAGAGCAATA | 228 |
| target site | TGgctggttccccaa | |
| | caTGCCTCACCCTCA | |
| | TCTA | |
| Off-site OS26 (OS1) | AAGCAGAGAAGGAAG | 229 |
| | CCctggaatgtgtag | |
| | aGAGGGCACCCTTAT | |
| | CTT | |
| Off-site OS28 (OS2) | TTCCAGAGAGCCAAG | 230 |
| | GAagctatttctatg | |
| | actaaTTCTCTTTCC | |
| | TCATCTA | |

EXAMPLE 5 (target sequences)

| TALE-BE V2 CD52-1 | TTTTGTCCTGAGAGT | 249 |
|---|---|---|
| | CCagtttgtatctgt | |
| | aGGAGGAGAAGTGGG | |
| | ATA | |
| TALE-BE V2 CD52-2 | TTTGTCCTGAGAGTC | 250 |
| | CAgtttgtatctgta | |
| | GGAGGAGAAGTGGGA | |
| | TA | |
| TALE-BE V2 CD52-3 | TTGTCCTGAGAGTCC | 251 |
| | AGtttgtatctgtaG | |
| | GAGGAGAAGTGGGAT | |
| | A | |
| TALE-BE V2 CD52-4 | TGGCTGGTGTCGTTT | 252 |
| | TGtcctgagagtcca | |
| | gtTTGTATCTGTAGG | |
| | AGGA | |
| EXAMPLE 6 (list of primers used to | | |
| amplify on and off-target sites) | | |
| TGFBRII F | TCATCCTGGAAGATG | 253 |
| | ACCGC | |
| TGFBRII R | TCATCCTGGAAGATG | 254 |
| | ACCGC | |
| OT1 F | GAGAAACCTGGCTTG | 255 |
| | TAGTG | |
| OT1_R | CATATTCATGAAAGG | 256 |
| | GAAGC | |
| OT2_F | ATCACTCATGGTCTG | 257 |
| | CATTAG | |
| OT2_R | CCAGACCTGGAAGAG | 258 |
| | TAGAT | |
| OT3_F | TGCGCTCGGCTATAA | 259 |
| | CGAT | |
| OT3_R | TGAAGTGACATTCAG | 260 |
| | ACCT | |
| OT4_F | GGAATTGGCAACTCA | 261 |
| | TCAGG | |
| OT4_R | ATTGCTACCGCACAA | 262 |
| | CTGG | |
| OT7_F | GAAGATCTACTCTGA | 263 |
| | ACCTC | |
| OT7_R | GTATTAGTCATTCTG | 264 |
| | CCTG | |
| OT17_F | GTGTTATATACACAC | 265 |
| | ATGGAC | |
| OT17_R | CTTGCAAATATGGTG | 266 |
| | CATCG | |

EXAMPLE 7 (target sequences)

| | | |
|---|---|---|
| TIGIT | TGTCACCTCTCCTCC | 289 |
| | ACcacggcacaagtg | |
| | ACCCAGGTCAACTGG | |
| | GA | |
| CISH (1) | TCGAGGAGGTGGCAG | 290 |
| | AGggtaccccagccc | |
| | agACAGAGAGTGAGC | |
| | CAAA | |
| CISH (2) | TGGTATTGGGGTTCC | 291 |
| | ATtacggccagcgag | |
| | gcCCGACAACACCTG | |
| | CAGA | |
| CISH (3) | TGCCTGCTGGGGCCT | 292 |
| | TCctcgaggaggtgg | |
| | caGAGGGTACCCCAG | |
| | CCCA | |
| CD38 (1) | TTTCCCGAGACCGTC | 293 |
| | CTggcgcgatgcgtc | |
| | AAGTACACTGAAATT | |
| | CA | |
| CD38 (2) | TGGAGCCCTATGGCC | 294 |
| | AActgcgagttcagc | |
| | CCGGTGTCCGGGGAC | |
| | AA | |
| IgH (1) | TGATATGTGTCTGGA | 295 |
| | ATtgaggccaaagca | |
| | AGCTCAGCTAAGAAA | |
| | TA | |
| IgH (2) | TGAGTGATATGTGTC | 296 |
| | TGgaattgaggccaa | |
| | AGCAAGCTCAGCTAA | |
| | GA | |
| GADPH (1) | TCTTATTCTAGGGTC | 297 |
| | TGgggcagaggggag | |
| | ggaAGCTGGGCTTGT | |
| | GTCAA | |
| GADPH (2) | TTTGCTTCCCGCTCA | 298 |
| | GAcgtcttgagtgct | |
| | ACAGGAAGCTGGCAC | |
| | CA | |

1. A Transcriptional Activator-like Effector (TALE) protein comprising a core binding domain comprising AvrBs3-like repeats, wherein said core binding domain is placed between N-terminal and C-terminal regions, wherein
said N-terminal region comprises a polypeptide sequence having at least 85%, sequence identity with SEQ ID NO: 1; and
said C-terminal region consists of a polypeptide sequence from 40 to 80 residues comprising a sequence having at least 85% identity with:
(SEQ ID NO: 2)
SIVAQLSRPDPALAALTNDHLVALACLGGRPALDAV [symbol] GL
(SEQ ID NO: 3)
SIVAQLSRPDPALAALTNDHLVALACLGGRPALDAV [symbol] GL
PHAPALI [symbol] RT,
or
(SEQ ID NO: 4)
SIVAQLSRPDPALAALTNDHLVALACLGGRPALDAV [symbol] G
LPHAPALI [symbol] RTNRRIPERTSH,
wherein X₁, X₂, and X₃, are a H (histidine) or a R (arginine) residue.

2. The transcriptional activator-like Effector (TALE) protein according to claim 1, wherein said C-terminal region comprises SEQ ID NO:2, SEQ ID NO: 3 or SEQ ID NO:4.

3. The transcriptional activator-like Effector (TALE) protein according to claim 1, wherein at least one of said AvrBs3-like repeats comprises D (aspartic acid) residues at positions 4 and 32 with respect to any of the canonical sequence of AvrBs3 of SEQ ID NO: 31 to 34.

4. The transcriptional activator-like Effector (TALE) protein according to claim 3, wherein at least 2 of said AvrBs3-like repeats comprise D (aspartic acid) residues at positions 4 and 32 or wherein said at least one AvrBs3-like repeat(s) is (are) further mutated in 1 to 5 amino acid positions in addition to D4 and D32.

5. (canceled)

6. The transcriptional activator-like Effector (TALE) protein according to claim 1, wherein at least one of said AvrBs3-like repeats comprises one of the sequences:
(SEQ ID NO: 5)
LTPDQVVAIASX₄X₅GGKQALETVQRLLPVLCQDHG,
(SEQ ID NO: 6)
LTPDQVVAIASX₄X₅GGKQALETVQALLPVLCQDHG,
(SEQ ID NO: 7)
LTPDQVVAIASX₄X₅GGKQALETVQQLLPVLCQDHG,
or
(SEQ ID NO: 8)
LTPDQLVAIASX₄X₅GGKQALETVQRLLPVLCQDHG,
(SEQ ID NO: 9)
LTPDQMVAIASX₄X₅GGKQALETVQRLLPVLCQDHG,
(SEQ ID NO: 10)
LTPDQVVAIASX₄X₅GGKQALETVQRLLPVLCQDQG,
(SEQ ID NO: 11)
LTLDQVVAIASX₄X₅GGKQALETVQRLLPVLCQDHG,
wherein X₄X₅ is an amino acid forming a variable di-residue.

7. (canceled)

8. (canceled)

9. The activator-like Effector (TALE) protein according to claim 1, wherein said TALE is fused to a nuclease domain to form a TALE-nuclease.

10. (canceled)

11. The TALE-nuclease according to claim 9, wherein said nuclease domain comprises a polypeptide sequence having that shows at least 85% identity, with SEQ ID NO: 109 (Fok1 catalytic domain) and wherein said nuclease domain has at least one amino acid substitution at a position corresponding to 13, 52, 57, 59, 61, 65, 84, 85, 88, 91, 92, 95, 98, 103, 109, 110, 111, 113, 119, 143, 148, 152, 158, 159, 160, 167, 169, 170, and 194 of SEQ ID NO: 109.

12. (canceled)

13. The transcriptional activator-like Effector (TALE) protein according to claim 1, wherein said TALE protein is fused to a deaminase domain to form a TALE-base editor.

14. The transcriptional activator-like Effector (TALE)-base editor according to claim 13, wherein said deaminase domain comprises a polypeptide sequence having at least 85% identity with SEQ ID NO: 134 or SEQ ID NO: 135.

15. The transcriptional activator-like Effector (TALE) protein according claim 1, wherein said TALE protein is fused to a transcriptional modulator domain to form a TALE-transcriptional modulator.

16. (canceled)

17. (canceled)

18. (canceled)

19. (canceled)

20. (canceled)

21. A method for producing a TALE protein for introducing a genetic modification into a polynucleotide sequence, said method comprising the steps of:
a) selecting a polynucleotide target sequence on which the genetic modification is intended;
b) assembling polynucleotide sequences encoding AvrBs3-like repeat(s) to form a polynucleotide encoding a TALE-binding domain to bind said selected polynucleotide target sequence;
c) fusing to said polynucleotide encoding the TALE-binding domain at least:
(1) a polynucleotide sequence encoding a N-terminal domain comprising a sequence having at least 85% identity with SEQ ID NO:1, and
(2) a polynucleotide sequence encoding a C-terminal domain consisting of a polypeptide sequence from 40 to 80 residues comprising a sequence having at least 85% identity with SEQ ID NO:2, SEQ ID NO:3 or SEQ ID NO:4; X₁, X₂, X₃ in these sequences representing R (arginine) or H (histidine).

22. The method according to claim 21, comprising an additional step d) of fusing to the polynucleotide encoding said C-terminal domain a polynucleotide sequence encoding a catalytic domain, such as of a nuclease or a deaminase.

23. The method according to claim 21, comprising an additional step of fusing a polynucleotide encoding a NLS (Nuclear Localization Signal) to the polynucleotide encoding said N-terminal domain.

24. The method according to claim 21, wherein said AvrBs3-like repeats comprise D at positions 4 (D4) and 32 (D32) in their polypeptide sequence.

25. (canceled)

26. The method according to claim 21, wherein said C-terminal domain is mutated to introduce 1 to 5 positively charged amino acids, selected from Lysine (K), Arginine (R) or histidine (H).

27. The method according to claim 22, wherein said nuclease catalytic domain in step d) is Fok-1.

28. The method according to claim 27, wherein at least one substitution is introduced in said Fok-1 catalytic domain at any one of the positions corresponding to 13, 52, 57, 59, 61, 65, 84, 85, 88, 91, 92, 95, 98, 103, 109, 110, 111, 113, 119, 143, 148, 152, 158, 159, 160, 167, 169, 170 and 194 of SEQ ID NO:109.

29. (canceled)

30. (canceled)

31. The method according to claim 21, further comprising a step of expressing the polynucleotide formed in step c) in a cell.

32. A polynucleotide encoding the TALE according to claim 1.

33. A vector or cell comprising a polynucleotide according to claim 32.

34. (canceled)

35. (canceled)
